(19) United States
(12) Patent Application Publication
(10) Pub. No.: US 2011/0104057 A1
(43) Pub. Date: May 5, 2011

(54) Novel [F-18]-labelled L-glutamic acid and L-glutamine derivatives (II), their use and processes for their preparation

(75) Inventors: Ludger Dinkelborg, Berlin (DE); Keith Graham, Berlin (DE); Mathias Berndt, Berlin (DE); Norman Koglin, Berlin (DE); Heribert Schmitt-Willich, Berlin (DE); Matthias Friebe, Berlin (DE); Lutz Lehmann, Berlin (DE)

(73) Assignee: BAYER SCHERING PHARMA AKTIENGESELLSCHAFT, BERLIN (DE)

(21) Appl. No.: 12/993,289

(22) PCT Filed: May 13, 2009
(86) PCT No.: PCT/EP09/03384

§ 371 Date: Jan. 19, 2011

(30) Foreign Application Priority Data
May 20, 2008 (DE) 08075508.5

(51) Int. Cl. A61K 49/08; A61K 31/4015; A61K 31/27; A61K 31/20; A61K 31/198; C07D 207/22; C07C 205/56; C07C 271/22; C07C 229/36; C07C 59/31; C07C 229/24; A61P 35/00

(52) U.S. Cl. 424/1.89; 514/423; 514/478; 514/482; 514/485; 514/558; 514/561; 514/567; 548/533; 560/22; 560/30; 560/160; 562/456; 562/505; 562/571

(57) ABSTRACT

What is described are the compounds and the synthesis of [F-18]-labelled L-glutamic acid, [F-18]-labelled L-glutamate, their derivatives of the formula (I) and their use.

### BACKGROUND

The invention relates to the subject matter referred to in the claims, i.e. [F-18]-labelled L-glutamic acid derivatives and [F-18]-labelled L-glutamine derivatives of the general formula I, and to their use and to processes for their preparation.

The early diagnosis of malignant tumour diseases plays an important role in the survival prognosis of a tumour patient. For this diagnosis, non-invasive diagnostic imaging methods are an important aid. In the last years, in particular the PET (Positron Emission Tomography) technology has been found to be particularly useful. The sensitivity and specificity of the PET technology depends essentially on the signal-giving substance (tracer) used and on its distribution in the body. In the hunt for suitable traces, one tries to make use of certain properties of tumours which differentiate tumour tissue from healthy surrounding tissue. The preferred commercial isotope used for PET applications is ¹⁸F. Owing to the short half-life of less than 2 hours, ¹⁸F is particularly demanding when it comes to the preparation of suitable tracers. This isotope does not allow for complicated long synthesis routes and purification procedures, since otherwise a considerable amount of the radioactivity of the isotope will already have faded away before the tracer can be used for diagnosis. Accordingly, it is frequently not possible to apply established synthesis routes for non-radioactive fluorinations to the synthesis of ¹⁸F tracers. Furthermore, the high specific activity of ¹⁸F [about 80 GBq/nmol) leads to very low substance amounts of [¹⁸F]-fluoride for the tracer synthesis, which in turn requires an extreme excess of precursor, making the result of a radio synthesis strategy based on a non-radioactive fluorination reaction unpredictable.

FDG ([F]-2-Fluorodeoxyglucose)-PET is a widely accepted and frequently used auxiliary in the diagnosis and further clinical monitoring of tumour disorders. Malignant tumours compete with the host organism for glucose as nutrient supply (Warburg O., Über den Stoffwechsel der Carcinomzelle [The metabolism of the carcinoma cell], Biochem. Zeitschrift 1924; 152: 309-339; Kellof G., Progress and Promise of FDG-PET Imaging for Cancer Patient Management and Oncologic Drug Development, Clin. Cancer Res. 2005; 11(8): 2785-2807). Compared to the surrounding cells of the normal tissue, tumour cells usually have an increased glucose metabolism. This is exploited when using fluorodeoxyglucose (FDG), a glucose derivative which is increasingly transported into the cells, where, however, it is metabolically captured as FDG 6-phosphate after phosphorylation (“Warburg effect”). Accordingly, ¹⁸F-labelled FDG is an effective tracer for detecting tumour disorders in patients using the PET technology. In the hunt for novel PET tracers, recently, amino acids have been employed increasingly for ¹⁸F PET imaging (for example (review): Eur. J. Nucl. Med. Mol. Imaging May 2002; 29(5): 681-90). Here, some of the ¹⁸F-labelled amino acids are suitable for measuring the rate of protein synthesis, but most other derivatives are suitable for measuring the direct cellular uptake in the tumour. Known ¹⁸F-labelled amino acids are derived, for example, from tyrosine amino acids, phenylalanine amino acids, proline amino acids, asparagine amino acids and unnatural amino acids (for example J. Nucl. Med. 1991; 32: 1338-1346, J. Nucl. Med. 1996; 37: 320-325, J. Nucl. Med. 2001; 42: 752-754 and J. Nucl. Med. 1999; 40: 331-338). Glutamic acid and glutamine as ¹⁸F-labelled derivatives are not known, whereas non-radioactive fluorinated glutamine and glutamic acid derivatives are known; thus, for example, those which carry fluorine in the γ-position (for example (review): Amino Acids April 2003; 24(3): 245-61) or in the β-position (for example Tetrahedron Lett. 1989; 30(14): 1799-1802, J. Org. Chem. 1989; 54(2): 498-500, Tetrahedron: Asymmetry 2001; 12(9): 1303-1312).

Glutamic acid derivatives having protective groups at the chemical functionalities and a leaving group in the β- or γ-position have already been reported in the past. Thus, there has been a report of glutamate having mesylate or bromide in the γ-position whose acid and amine functions were provided with ester and Z protective groups, respectively, (J. Chem. Soc. Perkin Trans. 1; 1986; 1323-1328) or, for example, of γ-chloroglutamic acid without protective groups (Synthesis; (1973); 44-46). There have also been various reports of similar derivatives where the leaving group was located in the β-position: for example Chem. Pharm. Bull.; 17; 5; (1969); 879-885, J. Gen. Chem. USSR (Engl. Transl.); 38; (1968); 1645-1648; Tetrahedron Lett., 27; 19; (1986); 2143-2144, Chem. Pharm. Bull.; EN; 17; 5; 1969; 873-878, Patent FR 1461184, Patent JP 13142.

The PET tracers currently used in tumour diagnosis have some undisputed disadvantages: thus, FDG is preferably accumulated in cells having an elevated glucose metabolism; however, under different pathological and physiological conditions, as also in elevated glucose metabolism in the cells and tissues involved, for example infection sites or wound healing (summarized in J. Nucl. Med. Technol. (2005), 33, 145-155). Frequently, it is still difficult to ascertain whether a lesion detected via FDG-PET is really of neoplastic origin or is the result of other physiological or pathological conditions of the tissue. Overall, the diagnosis by FDG-PET in oncology has a sensitivity of 84% and a specificity of 88% (Gambhir et al., “A tabulated summary of the FDG PET literature”, J. Nucl. Med. 2001, 42, 1-93S). The imaging of brain tumours, for example, is very difficult owing to the high accumulation of FDG in healthy brain tissue.

In some cases, the ¹⁸F-labelled amino acid derivatives currently known are well suited for the detection of tumours in the brain ((review): Eur. J. Nucl. Med. Mol. Imaging. 2002 May; 29(5): 681-90); however, in the case of other tumours, they are not able to compete with the imaging properties of the “Goldstandard” [¹⁸F]2-FDG. The metabolic accumulation and retention of the current F-18-labelled amino acids in tumour tissue is generally lower than of FDG. In addition, the preparation of isomerically pure F-18-labelled non-aromatic amino acids is chemically very demanding.

Similarly to glucose, for glutamic acid and glutamine, too, an increased metabolism in proliferating tumour cells has been described (Medina, J. Nutr. 1131: 2539S-2542S, 2001; Souba, Ann Surg 218: 715-728, 1993). The increased rate of protein and nucleic acid syntheses and the energy generation per se are thought to be the reasons for an increased glutamine consumption of tumour cells. The synthesis of corresponding C-11- and C-14-labelled compounds, which are thus identical to the natural substrate, has already been described in the literature (for example Antoni, Enzyme Catalyzed Synthesis of L-[4-C-11]aspartate and L-[5-C-11]glutamate. J. Labelled Compd. Radiopharm. 44; (4) 2001: 287-294 and Buchanan, The biosynthesis of showdomycin: studies with stable isotopes and the determination of principal precursors, J. Chem. Soc. Chem. Commun.; EN; 22; 1984; 1515-1517). First tests with the C-11-labelled compound indicate no significant accumulation in tumours.

It is an object of the present invention to provide novel compounds which, in [¹⁸F]-labelled form, are suitable for PET-based diagnosis.

### Invention

This object is achieved by the provision according to the invention of [¹⁸F]-labelled glutamic acid derivatives and [¹⁸F]-labelled glutamine derivatives of the general formula (I), including diastereomers and enantiomers:

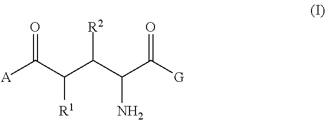

in which
A represents
a) hydroxyl,
b) branched or straight-chain C₁-C₅ alkoxy,
c) branched or straight-chain hydroxy C₁-C₅ alkoxy,
d) branched or straight-chain O—C₁-C₅ alkyl-(O—C₁-C₄ alkyl)ₙ-O—C₁-C₄ alkyl,
e) N(C₁-C₅ alkyl)₂,
f) NH₂,
g) N(H)-L,
h) O-L or
i) O—Z,
   G represents
a) hydroxyl,
b) O—Z,
c) branched or straight-chain O—C₁-C₅ alkyl,
d) branched or straight-chain O—C₂-C₅ alkenyl,
e) branched or straight-chain O—C₁-C₅ alkyl-(O—C₁-C₄ alkyl)ₙ-O—C₁-C₄ alkyl,
f) branched or straight-chain O—C₂-C₅ alkynyl or
g) triphenylmethoxy,
   R¹ and R² represent
a) hydrogen,
b) branched or straight-chain ¹⁸F—C₆-C₁₀ alkoxy,
c) branched or straight-chain ¹⁸F—C₆-C₁₀ alkyl,
d) branched or straight-chain ¹⁸F—C₆-C₁₀ alkenyl,
e) branched or straight-chain ¹⁸F—C₆-C₁₀ alkynyl,
f) hydroxyl,
g) branched or straight-chain C₁-C₅ alkyl or
h) branched or straight-chain C₁-C₅ alkoxy,
with the proviso that one of the substituents R¹ or R² contains exactly one ¹⁸F isotope and the respective other substituent contains no ¹⁸F isotope,
   L represents
a) branched or straight-chain C₁-C₅ alkyl,
b) branched or straight-chain C₁-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl, and
   Z represents a metal cation equivalent,
   where n=0, 1, 2 or 3 and
   where all possible diastereomers and enantiomers form part of the present subject matter of the invention.

Preferred compounds according to the invention of the formula (I) are distinguished in that

A represents
a) hydroxyl,
b) methoxy,
c) ethoxy,
d) propoxy,
e) NMe₂,
f) NEt₂,
g) NH₂,
h) N(H)-L,
i) O-L or
j) O—Z.

Further preferred compounds according to the invention of the formula (I) are distinguished in that

A represents
a) hydroxyl,
b) methoxy,
c) ethoxy,
d) NMe₂,
e) NH₂ or
f) N(H)-L.

Particularly preferred compounds according to the invention of the formula (I) are distinguished in that

A represents
a) hydroxyl,
b) branched or straight-chain C₁-C₅ alkoxy or
c) NH₂.

Preferred compounds according to the invention of the formula (I) are distinguished in that

A represents
hydroxyl.

Preferred compounds according to the invention of the formula (I) are distinguished in that

A represents
NH₂.

Preferred compounds according to the invention of the formula (I) are distinguished in that

A represents
ethoxy.

Preferred compounds according to the invention of the formula (I) are distinguished in that

G represents
a) hydroxyl,
b) branched or straight-chain O—C₁-C₄ alkyl or
c) O—C₂H₄—OMe.

Further preferred compounds according to the invention of the formula (I) are distinguished in that

G represents
a) hydroxyl or
b) branched or straight-chain O—C₁-C₄ alkyl.

Particularly preferred compounds according to the invention of the formula (I) are distinguished in that

G represents
a) hydroxyl,
b) methoxy or
c) ethoxy.

Particularly preferred compounds according to the invention of the formula (I) are distinguished in that

G represents
hydroxyl.

Preferred compounds according to the invention of the formula (I) are distinguished in that

R¹ and R² represent
a) hydrogen,
b) branched or straight-chain ¹⁸F—C₆-C₈ alkoxy,
c) branched or straight-chain ¹⁸F—C₆-C₈ alkyl,
d) branched or straight-chain ¹⁸F—C₆-C₈ alkenyl,
e) branched or straight-chain ¹⁸F—C₆-C₈ alkynyl or
f) hydroxyl,
   with the proviso that exactly one of the substituents R¹ or R² contains exactly one ¹⁸F-isotope and the respective other substituent is hydrogen.

A further particular subject matter of the invention are compounds of the general formula (I) in which

R¹ represents
a) branched or straight-chain ¹⁸F—C₆ alkoxy,
b) branched or straight-chain ¹⁸F—C₆ alkyl,
c) branched or straight-chain ¹⁸F—C₆ alkenyl or
d) branched or straight-chain ¹⁸F—C₆ alkynyl.

Straight-chain ¹⁸F—C₆ alkoxy is ¹⁸F-hexoxy.

Straight-chain ¹⁸F—C₆ alkyl is ¹⁸F-hexyl.

Straight-chain ¹⁸F—C₆ alkenyl is ¹⁸F-hexenyl.

Straight-chain ¹⁸F—C₆ alkynyl is ¹⁸F-hexynyl.

A further particular subject matter of the invention are compounds of the general formula I in which

R¹ represents ¹⁸F-hexoxy or ¹⁸F-hexyl and R² represents hydrogen.

Preferred compounds according to the invention of the formula (I) are distinguished in that R¹ and R² are selected from the group consisting of hydrogen, ¹⁸F-hexoxy, ¹⁸F-heptoxy, ¹⁸F-octoxy, ¹⁸F-nonoxy, ¹⁸F-decoxy, ¹⁸F-hexyl, ¹⁸F-heptyl, ¹⁸F-octyl, ¹⁸F-nonyl, ¹⁸F-decyl and may be interrupted by one to three oxygen atoms with the proviso that one of the substituents R¹ or R² contains exactly one ¹⁸F isotope and the respective other substituent is hydrogen.

Preferred compounds according to the invention of the formula (I) are distinguished in that

L represents
a) methyl,
b) ethyl,
c) propyl,
d) isopropyl,
e) —C₂H₄—OMe or
f) —C₂H₄—O—C₂H₄—OMe.

Particularly preferred compounds according to the invention of the formula (I) are distinguished in that

L represents
a) methyl or
b) ethyl.

Compounds according to the invention of the formula (I) which are likewise preferred are distinguished in that Z is selected from the group consisting of Na⁺, K⁺, Ca²⁺ and Mg²⁺. Z is preferably Na⁺.

All possible diastereomers and enantiomers of the preferred compounds of the formula (I) form part of the present subject matter of the invention.

Preferred compounds according to the invention of the formula (I) are distinguished in that formula (I) is

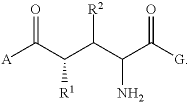

Compounds according to formula I for use as a medicament.

Compounds according to formula I for use for imaging in tumour disorders.

Use of compounds according to formula I for producing a medicament for imaging in tumour disorders.

The process for preparing the compounds according to the invention of the general formula (I) is distinguished in that
one or more protective groups present in a compound of the formula (II) is/are removed.

Particular preference is furthermore given to each individual compound from the following group, all possible diastereomers and enantiomers being part of the present subject matter of the invention.

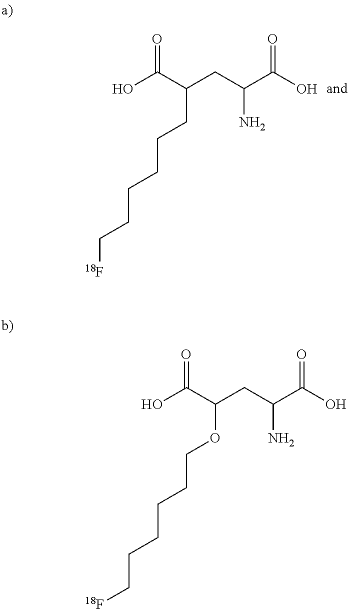

At physiological pH 7.4, the compounds according to the invention of the formula (I) may also be present as zwitterions or salts, as is known to those skilled in the art.

According to a further aspect, the present invention thus relates to compounds of the formula (II):

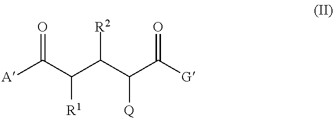

A′ represents
a) hydroxyl,
b) branched or straight-chain C₁-C₅ alkoxy,
c) branched or straight-chain hydroxy C₁-C₅ alkoxy,
d) branched or straight-chain O—C₁-C₅ alkyl-(O—C₁-C₄ alkyl)ₙ-O—C₁-C₄ alkyl,
e) N(C₁-C₅ alkyl)₂,
f) NH₂,
g) N(H)-L′, or
h) O-L′,
   G′ represents
a) hydroxyl,
b) O—Z′,
c) branched or straight-chain O—C₁-C₅ alkyl,
d) branched or straight-chain O—C₁-C₅ alkenyl,
e) branched or straight-chain O—C₁-C₅ alkyl-(O—C₁-C₄ alkyl)ₙ-O—C₁-C₄ alkyl,
f) branched or straight-chain O—C₁-C₅ alkynyl or
g) triphenylmethoxy,
   R¹ and R² represent
a) hydrogen,
b) branched or straight-chain ¹⁸F—C₆-C₁₀ alkoxy,
c) branched or straight-chain ¹⁸F—C₆-C₁₀ alkyl,
d) branched or straight-chain ¹⁸F—C₆-C₁₀ alkenyl,
e) branched or straight-chain ¹⁸F—C₆-C₁₀ alkynyl,
f) hydroxyl,
g) branched or straight-chain C₁-C₅ alkyl or
h) branched or straight-chain C₁-C₅ alkoxy,
   with the proviso that exactly one of the substituents R¹ or R² contains exactly one ¹⁸F isotope and the respective other substituent contains no ¹⁸F isotope,
   Q represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
e) N(H)-2,2,2-trichloroethoxycarbonyl,
f) N(H)-1,1-dimethylpropynyl,
g) N(H)-1-methyl-1-phenylethoxycarbonyl,
h) N(H)-1-methyl-1-(4-biphenylyl)ethoxycarbonyl,
i) N(H)-cyclobutylcarbonyl,
j) N(H)-1-methylcyclobutylcarbonyl,
k) N(H)-vinylcarbonyl,
l) N(H)-allylcarbonyl,
m) N(H)-adamantylcarbonyl,
n) N(H)-diphenylmethylcarbonyl,
o) N(H)-cinnamylcarbonyl,
p) N(H)-formyl,
q) N(H)-benzoyl,
r) N(H)-trityl,
s) N(H)-p-methoxydiphenylmethyl,
t) N(H)-di(p-methoxyphenyl)phenylmethyl,
u)

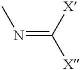

or
v) N-(tert-butoxycarbonyl)₂,
   L′ represents
a) branched or straight-chain C₁-C₅ alkyl,
b) branched or straight-chain C₁-C₅ alkenyl,
c) branched or straight-chain C₁-C₅ alkyl-(O—C₁-C₄ alkyl)ₙ-O—C₁-C₄ alkyl or
d) branched or straight-chain C₁-C₅ alkynyl,
   X′ and X″ independently of one another represent
a) branched or straight-chain C₁-C₅ alkyl,
b) substituted or unsubstituted aryl,
c) substituted or unsubstituted aralkyl or
d) substituted or unsubstituted heteroaryl,
   Z′ represents a metal cation equivalent,
   where n=0, 1, 2 or 3 and all possible diastereomers and enantiomers form part of the present subject matter of the invention.

Preferred compounds according to the invention of the formula (II) are distinguished in that

A′ represents
a) hydroxyl,
b) methoxy,
c) ethoxy,
d) propoxy,
e) NMe₂,
f) NEt₂,
g) NH₂,
h) N(H)-L,
i) O-L or
j) O—Z.

Further preferred compounds according to the invention of the formula (II) are distinguished in that

A′ represents
a) hydroxyl,
b) methoxy,
c) ethoxy,
d) NMe₂,
e) NH₂ or
f) N(H)-L.

Particularly preferred compounds according to the invention of the formula (II) are distinguished in that

A′ represents
a) hydroxyl,
b) branched or straight-chain C₁-C₅ alkoxy or
c) NH₂.

Preferred compounds according to the invention of the formula (II) are distinguished in that

A′ represents
hydroxyl.

Preferred compounds according to the invention of the formula (II) are distinguished in that

A′ represents
NH₂.

Preferred compounds according to the invention of the formula (II) are distinguished in that

A′ represents
ethoxy.

Preferred compounds according to the invention of the formula (II) are distinguished in that

A′ represents
tert-butoxy.

Preferred compounds according to the invention of the formula (II) are distinguished in that

G′ represents
a) hydroxyl,
b) branched or straight-chain O—C₁-C₄ alkyl or
c) O—C₂H₄—OMe.

Further preferred compounds according to the invention of the formula (II) are distinguished in that

G′ represents
a) hydroxyl or
b) branched or straight-chain O—C₁-C₄ alkyl.

Particularly preferred compounds according to the invention of the formula (II) are distinguished in that

G′ represents
a) hydroxyl,
b) methoxy or
c) ethoxy.

Particularly preferred compounds according to the invention of the formula (II) are distinguished in that

G′ represents
methoxy.

Particularly preferred compounds according to the invention of the formula (II) are distinguished in that

G′ represents
tert-butoxy.

Preferred compounds according to the invention of the formula (II) are distinguished in that

R² and R² represent
a) hydrogen,
b) branched or straight-chain ¹⁸F—C₆-C₈ alkoxy,
c) branched or straight-chain ¹⁸F—C₆-C₈ alkyl,
d) branched or straight-chain ¹⁸F—C₆-C₈ alkenyl,
e) branched or straight-chain ¹⁸F—C₆-C₈ alkynyl,
   with the proviso that exactly one of the substituents R¹ or R² contains exactly one ¹⁸F-isotope and the respective other substituent is hydrogen.

A further particular subject matter of the invention are compounds of the general formula II in which

R¹ represents
a) branched or straight-chain ¹⁸F—C₆ alkoxy,
b) branched or straight-chain ¹⁸F—C₆ alkyl,
c) branched or straight-chain ¹⁸F—C₆ alkenyl or
d) branched or straight-chain ¹⁸F—C₆ alkynyl.

Straight-chain ¹⁸F—C₆ alkoxy is ¹⁸F-hexoxy. Straight-chain ¹⁸F—C₆ alkyl is ¹⁸F-hexyl. Straight-chain ¹⁸F—C₆ alkenyl is ¹⁸F-hexenyl. Straight-chain ¹⁸F—C₆ alkynyl is ¹⁸F-hexynyl.

A further particular subject matter of the invention are compounds of the general formula II in which

R¹ represents ¹⁸F-hexoxy or ¹⁸F-hexyl and R² represents hydrogen.

Preferred compounds according to the invention of the formula (II) are distinguished in that R¹ and R² are selected from the group consisting of hydrogen, ¹⁸F-hexoxy, ¹⁸F-heptoxy, ¹⁸F-octoxy, ¹⁸F-nonoxy, ¹⁸F-decoxy, ¹⁸F-hexyl, ¹⁸F-heptyl, ¹⁸F-octyl, ¹⁸F-nonyl, ¹⁸F-decyl and may be interrupted by one to three oxygen atoms with the proviso that one of the substituents R¹ or R² contains exactly one ¹⁸F isotope and the respective other substituent is hydrogen.

Preferred compounds according to the invention of the formula (II) are distinguished in that

L′ represents
a) methyl,
b) ethyl,
c) propyl,
d) isopropyl,
e) —C₂H₄—OMe or
f) —C₂H₄—O—C₂H₄—OMe.

Particularly preferred compounds according to the invention of the formula (II) are distinguished in that

L′ represents
a) methyl or
b) ethyl.

Compounds according to the invention of the formula (II) which are likewise preferred are distinguished in that Z′ is selected from the group consisting of Na⁺, K⁺, Ca²⁺ and Mg²⁺. Z′ is preferably Na⁺.

All possible diastereomers and enantiomers of the preferred compounds of the formula (II) form part of the present subject matter of the invention.

Preferred compounds according to the invention of the formula (II) are distinguished in that

Q represents
a) N(H)-tert-butoxycarbonyl,
b) N(H)-benzyloxycarbonyl,
c) N-(tert-butoxycarbonyl)₂ or
d)

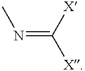

Further preferred compounds according to the invention of the formula (II) are distinguished in that

Q represents
a) N(H)-tert-butoxycarbonyl,
b) N-(tert-butoxycarbonyl)₂ or
c)

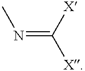

Particularly preferred compounds according to the invention of the formula (II) are distinguished in that

Q represents
a) N(H)-tert-butoxycarbonyl or
b)

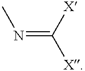

Particularly preferred compounds according to the invention of the formula (II) are distinguished in that Q represents N(H)-tert-butoxycarbonyl.

Preferred compounds according to the invention of the formula (II) are distinguished in that

L′ represents
a) methyl,
b) ethyl,
c) propyl,
d) isopropyl,
e) —C₂H₄—OMe or
f) —C₂H₄—O—C₂H₄—OMe.

Preferred compounds according to the invention of the formula (II) are distinguished in that

X′ and X″ independently of one another represent
a) branched or straight-chain C₁-C₅ alkyl,
b) substituted or unsubstituted aryl or
c) substituted or unsubstituted aralkyl.

Further preferred compounds according to the invention of the formula (II) are distinguished in that

X′ and X″ independently of one another represent
a) branched or straight-chain C₁-C₅ alkyl or
b) substituted or unsubstituted aryl.

Particularly preferred compounds according to the invention of the formula (II) are distinguished in that X′ and X″ represent phenyl or represent phenyl which is substituted in the 2-position.

All possible diastereomers and enantiomers of the preferred compounds according to the invention of formula (II) form part of the present subject matter of the invention.

Compounds according to formula II for use as a medicament.

Compounds according to formula II for use for imaging in tumour disorders.

Use of compounds according to formula II for producing a medicament for imaging in tumour disorders.

Particular preference is furthermore given to each individual compound from the following group, all possible diastereomers and enantiomers being part of the present subject matter of the invention

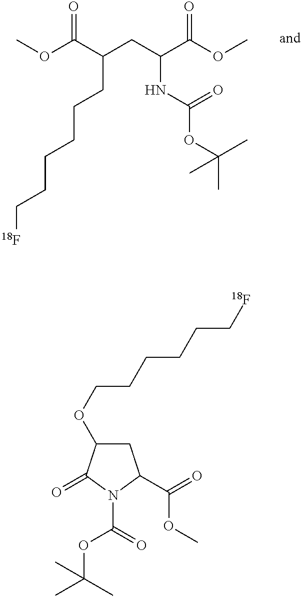

The process for preparing the compounds of the general formula (II) according to the invention is distinguished in that the plurality of the compounds according to formula (II) can be formed from a precursor compound of the compound of the formula (III) following introduction of the ¹⁸F-isotope.

According to a third aspect, the present invention relates to compounds of the formula (III):

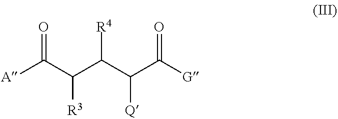

in which
A″ represents
a) branched or straight-chain C₁-C₅ alkoxy,
b) branched or straight-chain hydroxy C₁-C₅ alkoxy,
c) branched or straight-chain O—C₁-C₅ alkyl-(O—C₁-C₄ alkyl)ₙ-O—C₁-C₄ alkyl,
d) N(C₁-C₅ alkyl)₂,
e) NH₂,
f) N(H)-L″, or
g) O-L″,
   G″ represents
a) O—Z″,
b) branched or straight-chain O—C₁-C₅ alkyl,
c) branched or straight-chain O—C₂-C₅ alkenyl,
d) branched or straight-chain O—C₁-C₅ alkyl-(O—C₁-C₄ alkyl)ₙ-O—C₁-C₄ alkyl,
e) branched or straight-chain O—C₂-C₅ alkynyl or
f) triphenylmethoxy,
   R³ and R⁴ represent
a) hydrogen,
b) branched or straight-chain E-C₆-C₁₀ alkoxy,
c) branched or straight-chain E-C₆-C₁₀ alkyl,
d) branched or straight-chain E-C₆-C₁₀ alkenyl,
e) branched or straight-chain E-C₆-C₁₀ alkynyl,
f) hydroxyl,
g) branched or straight-chain C₁-C₅ alkyl or
h) branched or straight-chain C₁-C₅ alkoxy,
with the proviso that exactly one of the substituents R³ or R⁴ contains an E and the respective other substituent contains no E,
   E represents a leaving group,
   Q′ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
g) N(H)-2,2,2-trichloroethoxycarbonyl,
h) N(H)-1,1-dimethylpropynyl,
i) N(H)-1-methyl-1-phenylethoxycarbonyl,
j) N(H)-1-methyl-1-(4-biphenylyl)ethoxycarbonyl,
k) N(H)-cyclobutylcarbonyl,
l) N(H)-1-methylcyclobutylcarbonyl,
m) N(H)-vinylcarbonyl,
n) N(H)-allylcarbonyl,
o) N(H)-adamantylcarbonyl,
p) N(H)-diphenylmethylcarbonyl,
q) N(H)-cinnamylcarbonyl,
r) N(H)-formyl,
s) N(H)-benzoyl,
t) N(H)-trityl,
u) N(H)-p-methoxyphenyldiphenylmethyl,
v) N(H)-di(p-methoxyphenyl)phenylmethyl,
w)

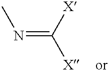

x) N-(tert-butoxycarbonyl)₂,
   L″ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
   X′ and X″ independently of one another represent
a) branched or straight-chain C₁-C₅ alkyl,
b) substituted or unsubstituted aryl,
c) substituted or unsubstituted alkylaryl or
d) substituted or unsubstituted heteroaryl, and
   Z″ represents a metal cation equivalent,
   where n=0, 1, 2 or 3 and
   all possible diastereomers and enantiomers form part of the present subject matter of the invention.

Preferred compounds according to the invention of the formula (III) are distinguished in that

A″ represents
a) branched or straight-chain C₁-C₅ alkoxy or
b) NH₂.

Further preferred compounds according to the invention of the formula (III) are distinguished in that

A″ represents
a) methoxy,
b) ethoxy
c) NH₂ or
d) tert-butoxy.

Preferred compounds according to the invention of the formula (III) are distinguished in that

A″ represents
methoxy.

Preferred compounds according to the invention of the formula (III) are distinguished in that

A″ represents
NH₂.

Preferred compounds according to the invention of the formula (III) are distinguished in that

A″ represents
ethoxy.

Preferred compounds according to the invention of the formula (III) are distinguished in that

A″ represents
tert-butoxy.

Preferred compounds according to the invention of the formula (III) are distinguished in that

G″ represents
a) branched or straight-chain O—C₁-C₅ alkyl,
b) branched or straight-chain O—C₁-C₅ alkenyl,
c) branched or straight-chain O—C₁-C₅ alkyl-(O—C₁-C₄ alkyl)ₙ-O—C₁-C₄ alkyl or
e) triphenylmethoxy.

Further preferred compounds according to the invention of the formula (III) are distinguished in that

G″ represents
a) methoxy,
b) ethoxy or
c) tert-butoxy.

Particularly preferred compounds according to the invention of the formula (III) are distinguished in that

G″ represents
a) tert-butoxy.

Preferred compounds according to the invention of the formula (III) are distinguished in that

R³ and R⁴ represent
a) hydrogen,
b) branched or straight-chain E-C₆-C₁₀ alkoxy,
c) branched or straight-chain E-C₆-C₁₀ alkyl,
d) branched or straight-chain E-C₆-C₁₀ alkenyl or
e) branched or straight-chain E-C₆-C₁₀ alkynyl,
   with the proviso that exactly one of the substituents R³ or R⁴ contains an E and the respective other substituent is hydrogen.

Preferred compounds according to the invention of the formula (III) are distinguished in that

R³ and R⁴ represent
a) hydrogen,
b) branched or straight-chain E-C₆-C₈ alkoxy,
c) branched or straight-chain E-C₆-C₈ alkyl,
d) branched or straight-chain E-C₆-C₈ alkenyl or
e) branched or straight-chain E-C₆-C₈ alkynyl,
f) hydroxyl,
g) branched or straight-chain C₁-C₈ alkyl or
h) branched or straight-chain C₁-C₈ alkoxy,
   with the proviso that exactly one of the substituents R³ or R⁴ contains an E and the respective other substituent does not contain an E.

A further particular subject matter of the invention are compounds of the general formula III in which

R³ represent
a) branched or straight-chain E-C₆ alkoxy,
b) branched or straight-chain E-C₆ alkyl,
c) branched or straight-chain E-C₆ alkenyl or
d) branched or straight-chain E-C₆ alkynyl.

Straight-chain E-C₆ alkoxy is E-hexoxy.

Straight-chain E-C₆ alkyl is E-hexyl.

Straight-chain E-C₆ alkenyl is E-hexenyl.

Straight-chain E-C₆ alkynyl is E-hexynyl.

A further particular subject matter of the invention are compounds of the general formula III in which

R³ represents E-hexoxy or E-hexyl and R⁴ represents hydrogen.

E is a leaving group evident or known to the person skilled in the art and mentioned or described, for example, in Synthese (1982), pages 85-125, Table 2, page 86; Carey and Sundberg, Organische Synthese, (1995), pages 279-281, Table 5.8; or Netscher, Recent Res. Dev. Org. Chem., 2003, 7, 71-83, schemes 1, 2, 10 and 15 or in Jerry March, Advanced Organic Chemistry, 4th edition, John Wiley and Sons, pp. 351-56 and 642-653), without being limited thereto.

Preferred compounds according to the invention of the formula (III) are distinguished in that E represents
halogen or
sulphonyloxy.

Preferred halogens are iodo, bromo and chloro.

Preferred sulphonyloxy are methanesulphonyloxy, trifluoromethanesulphonyloxy, nonafluorobutyloxy, tosyloxy and nosyloxy.

Preferred compounds according to the invention of the formula (III) are distinguished in that

E represents
a) chloro,
b) bromo,
c) methanesulphonyloxy,
d) trifluoromethanesulphonyloxy,
e) nonafluorobutyloxy,
f) tosyloxy or
g) iodo.

Preferred compounds according to the invention of the formula (III) are distinguished in that E represents
a) chloro,
b) bromo,
c) methanesulphonyloxy,
d) trifluoromethanesulphonyloxy,
e) tosyloxy or
f) iodo.

Further preferred compounds according to the invention of the formula (III) are distinguished in that E represents
a) chloro,
b) bromo,
c) methanesulphonyloxy,
d) trifluoromethanesulphonyloxy,
e) tosyloxy or
f) iodo.

Particularly preferred compounds according to the invention of the formula (III) are distinguished in that E represents
a) bromo, or
b) methanesulphonyloxy.

Preferred compounds according to the invention of the formula (III) are distinguished in that Q′ represents
a) N(H)-tert-butoxycarbonyl,
b) N(H)-benzyloxycarbonyl,
c) N(H)-trityl or
d)

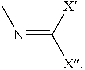

Further preferred compounds according to the invention of the formula (III) are distinguished in that Q′ represents
a) N(H)-tert-butoxycarbonyl or
b)

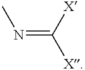

Preferred compounds according to the invention of the formula (III) are distinguished in that

L″ represents
a) branched or straight-chain C₁-C₅ alkyl,
b) branched or straight-chain C₂-C₅ alkenyl or
c) branched or straight-chain C₂-C₅ alkynyl.

Preferred compounds according to the invention of the formula (III) are distinguished in that

L″ represents
a) methyl,
b) ethyl,
c) propyl,
d) isopropyl,
e) —C₂H₄—OMe or
f) —C₂H₄—O—C₂H₄—OMe.

Further preferred compounds according to the invention of the formula (III) are distinguished in that

L″ represents
a) methyl, or
b) ethyl.

Preferred compounds according to the invention of the formula (III) are distinguished in that

X′ and X″ independently of one another represent
a) branched or straight-chain C₁-C₅ alkyl,
b) substituted or unsubstituted aryl or
c) aralkyl.

Further preferred compounds according to the invention of the formula (III) are distinguished in that

X′ and X″ independently of one another represent
a) branched or straight-chain C₁-C₅ alkyl or
b) substituted or unsubstituted aryl.

Particularly preferred compounds according to the invention of the formula (III) are distinguished in that

X′ and X″ represent phenyl or phenyl which is substituted in the 2-position.

All possible diastereomers and enantiomers of the preferred compounds according to formula (III) form part of the present subject matter of the invention.

All possible diastereomers and enantiomers of the preferred compounds according to formula (III) form part of the present subject matter of the invention.

Preferred compounds according to the invention of the formula (III) are distinguished in that that formula (III) is

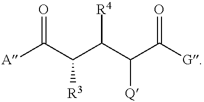

Compounds according to the invention of the formula (III) which are likewise preferred are distinguished in that Z′ is selected from the group consisting of NA⁺, K⁺, Ca²⁺ and Mg²⁺.

Z′ is preferably Na⁺.

According to a further aspect, the present invention relates to compounds of the formula (IV):

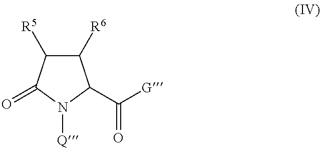

in which
G′″ represents
a) branched or straight-chain O—C₁-C₅ alkyl,
b) branched or straight-chain O—C₂-C₅ alkenyl,
c) branched or straight-chain O—C₁-C₅ alkyl-(O—C₁-C₄ alkyl)ₙ-O—C₁-C₄ alkyl,
d) branched or straight-chain O—C₂-C₅ alkynyl or
e) triphenylmethoxy,
   R⁵ and R⁶ represent
a) hydrogen,
b) hydroxyl,
c) branched or straight-chain C₁-C₅ alkyl,
d) branched or straight-chain C₁-C₅ alkoxy or
e) R⁷-E′,
   with the proviso that exactly one of the substituents R⁵ or R⁶ contains an E′ and the respective other substituent contains no E′,
   E′ represents a leaving group,
   R⁷ represents
a) branched or straight-chain C₆-C₁₀ alkoxy,
b) branched or straight-chain C₆-C₁₀ alkyl,
c) branched or straight-chain C₆-C₁₀ alkenyl or
d) branched or straight-chain C₆-C₁₀ alkynyl,
   Q′″ represents
a) N-tert-butoxycarbonyl
b) N-allyloxycarbonyl,
c) N-benzyloxycarbonyl,
d) N-ethoxycarbonyl,
e) N-methoxycarbonyl,
f) N-propoxycarbonyl,
g) N-2,2,2-trichloroethoxycarbonyl,
h) hydrogen,
i) N-1-methyl-1-phenylethoxycarbonyl,
j) N-1-methyl-1-(4-biphenylyl)ethoxycarbonyl,
k) N-cyclobutylcarbonyl,
l) N-1-methylcyclobutylcarbonyl,
m) N-vinylcarbonyl,
n) N-allylcarbonyl,
o) N-adamantylcarbonyl,
p) N-diphenylmethylcarbonyl,
q) N-cinnamylcarbonyl,
r) N-formyl,
s) N-benzoyl,
t) N(H)-trityl,
u) N(H)-p-methoxyphenyldiphenylmethyl,
v) N(H)-di(p-methoxyphenyl)phenylmethyl,
w)

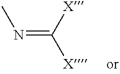

x) N-(tert-butoxycarbonyl)₂,
   X′″ and X″″ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
   where n=0, 1, 2 or 3 and
   all diastereomers and enantiomers are included.

Particularly preferred compounds according to the invention of the formula (IV) are distinguished in that G′″ represents
a) branched or straight-chain O—C₁-C₅ alkyl,
b) branched or straight-chain O—C₂-C₅ alkenyl,
c) branched or straight-chain O—C₁-C₅ alkyl-(O—C₁-C₄ alkyl)ₙ-O—C₁-C₄ alkyl or
e) triphenylmethoxy.

Further preferred compounds according to the invention of the formula (IV) are distinguished in that G′″ represents
a) methoxy,
b) ethoxy or
c) tert-butoxy.

Particularly preferred compounds according to the invention of the formula (IV) are distinguished in that G′″ represents
a) methoxy.

E′ is a leaving group evident or known to the person skilled in the art and mentioned or described, for example, in Synthese (1982), pages 85-125, Table 2, page 86; Carey and Sundberg, Organische Synthese, (1995), pages 279-281, Table 5.8; or Netscher, Recent Res. Dev. Org. Chem., 2003, 7, 71-83, schemes 1, 2, 10 and 15 or in Jerry March, Advanced Organic Chemistry, 4th edition, John Wiley and Sons, pp. 351-56 and 642-653), without being limited thereto.

Preferred compounds according to the invention of the formula (IV) are distinguished in that E′ represents
halogen or
sulphonyloxy.

Preferred halogens are iodo, bromo and chloro.

Preferred sulphonyloxy are methanesulphonyloxy, trifluoromethanesulphonyloxy, nonafluorobutyloxy, tosyloxy and nosyloxy.

Preferred compounds according to the invention of the formula (IV) are distinguished in that E′ represents
a) chloro,
b) bromo,
c) methanesulphonyloxy,
d) trifluoromethanesulphonyloxy,
e) nonafluorobutyloxy,
f) tosyloxy or
g) iodo.

Preferred compounds according to the invention of the formula (IV) are distinguished in that E′ represents
a) chloro,
b) bromo,
c) methanesulphonyloxy,
d) trifluoromethanesulphonyloxy,
e) tosyloxy or
f) iodo.

Further preferred compounds according to the invention of the formula (IV) are distinguished in that E′ represents
a) bromo,
b) methanesulphonyloxy,
c) trifluoromethanesulphonyloxy,
d) tosyloxy or
e) iodo.

Particularly preferred compounds according to the invention of the formula (IV) are distinguished in that E′ represents
a) bromo or
b) methanesulphonyloxy.

Preferred compounds according to the invention of the formula (IV) are distinguished in that

Q′ represents
a) N(H)-tert-butoxycarbonyl,
b) N(H)-benzyloxycarbonyl,
c) N(H)-trityl or
d)

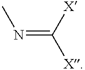

Further preferred compounds according to the invention of the formula (IV) are distinguished in that Q′ represents
a) N(H)-tert-butoxycarbonyl or
b)

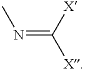

Preferred compounds according to the invention of the formula (IV) are distinguished in that

X′ and X″ independently of one another represent
a) branched or straight-chain C₁-C₅ alkyl,
b) substituted or unsubstituted aryl or
c) substituted or unsubstituted aralkyl.

Further preferred compounds according to the invention of the formula (IV) are distinguished in that

X′ and X″ independently of one another represent
a) branched or straight-chain C₁-C₅ alkyl or
b) substituted or unsubstituted aryl.

Particularly preferred compounds according to the invention of the formula (IV) are distinguished in that X′ and X″ represent phenyl or phenyl which is substituted in the 2-position.

All possible diastereomers and enantiomers of the preferred compounds of the formula (IV) form part of the present subject matter of the invention.

Preferred compounds according to the invention of the formula (IV) are distinguished in that formula (IV) is

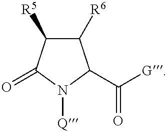

Compounds according to the invention of the formula (IV) which are likewise preferred are distinguished in that Z′ is selected from the group consisting of Na⁺, K⁺, Ca²⁺ and Mg²⁺.

Z′ is preferably Na⁺.

According to a further aspect, the present invention relates to the use of compounds of the formula (IV) for preparing compounds of the formula (I) or (II):

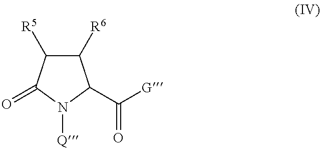

in which
G′″ represents
a) branched or straight-chain O—C₁-C₅ alkyl,
b) branched or straight-chain O—C₁-C₅ alkenyl,
c) branched or straight-chain O—C₁-C₅ alkyl-(O—C₁-C₄ alkyl)ₙ-O—C₁-C₄ alkyl,
d) branched or straight-chain O—C₁-C₅ alkynyl or
e) triphenylmethoxy,
   R⁵ and R⁶ represent
a) hydrogen,
b) hydroxyl,
c) branched or straight-chain C₁-C₅ alkyl,
d) branched or straight-chain C₁-C₅ alkoxy or
e) R⁷-E′,
   with the proviso that exactly one of the substituents R⁵ or R⁶ contains an E′ and the respective other substituent contains no E′,
   E′ represents a leaving group,
   R⁷ represents
a) branched or straight-chain C₆-C₁₀ alkoxy,
b) branched or straight-chain C₆-C₁₀ alkyl,
c) branched or straight-chain C₆-C₁₀ alkenyl or
d) branched or straight-chain C₆-C₁₀ alkynyl,
   Q′″ represents
a) N-tert-butoxycarbonyl
b) N-allyloxycarbonyl,
c) N-benzyloxycarbonyl,
d) N-ethoxycarbonyl,
e) N-methoxycarbonyl,
f) N-propoxycarbonyl,
g) N-2,2,2-trichloroethoxycarbonyl,
h) hydrogen,
i) N-1-methyl-1-phenylethoxycarbonyl,
j) N-1-methyl-1-(4-biphenylyl)ethoxycarbonyl,
k) N-cyclobutylcarbonyl,
l) N-1-methylcyclobutylcarbonyl,
m) N-vinylcarbonyl,
n) N-allylcarbonyl,
o) N-adamantylcarbonyl,
p) N-diphenylmethylcarbonyl,
q) N-cinnamylcarbonyl,
r) N-formyl,
s) N-benzoyl,
t) N(H)-trityl,
u) N(H)-p-methoxyphenyldiphenylmethyl,
v) N(H)-di(p-methoxyphenyl)phenylmethyl,
w)

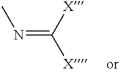

x) N-(tert-butoxycarbonyl)₂,
   X′″ and X″″ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
   where n=0, 1, 2 or 3 and
   all diastereomers and enantiomers are included.

According to a further aspect, the present invention relates to imaging kits comprising compounds of the general formula III or IV.

According to a further aspect, the present invention relates to pharmaceutical compositions comprising compounds of the general formula I, II, III or IV and suitable pharmaceutical carrier substances.

According to a further aspect, the present invention relates to compounds of the general formula V

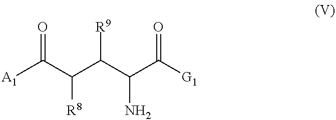

in which
A₁ represents
a) hydroxyl,
b) branched or straight-chain C₁-C₅ alkoxy,
c) branched or straight-chain hydroxy C₁-C₅ alkoxy,
d) branched or straight-chain O—C₁-C₅ alkyl-(O—C₁-C₄ alkyl)ₙ-O—C₁-C₄ alkyl,
e) N(C₁-C₅ alkyl)₂,
f) NH₂,
g) N(H)-L₁,
h) O-L₁ or
i) O—Z₁,
   G₁ represents
a) hydroxyl,
b) O—Z₁,
c) branched or straight-chain O—C₁-C₅ alkyl,
d) branched or straight-chain O—C₂-C₅ alkenyl,
e) branched or straight-chain O—C₁-C₅ alkyl-(O—C₁-C₄ alkyl)ₙ-O—C₁-C₄ alkyl,
f) branched or straight-chain O—C₁-C₅ alkynyl or
g) triphenylmethoxy,
   R⁸ and R⁹ represent
a) hydrogen,
b) substituted or unsubstituted ¹⁸F—C₆-C₁₀ mono- or bicyclic aryl,
c) substituted or unsubstituted alkylene-C₆-C₁₀ mono- or bicyclic aryl-¹⁸F,
d) substituted or unsubstituted ¹⁸F—C₅-C₁₀ mono- or bicyclic heteroaryl,
e) substituted or unsubstituted alkylene-C₅-C₁₀ mono- or bicyclic heteroaryl-¹⁸F,
f) substituted or unsubstituted ¹⁸F—C₃-C₆ cycloalkyl,
g) substituted or unsubstituted alkylene-C₃-C₆ cycloalkyl-¹⁸F,
h) hydroxyl,
i) branched or straight-chain C₁-C₅ alkyl,
j) branched or straight-chain C₁-C₅ alkoxy,
k) substituted or unsubstituted O-alkylene-C₆-C₁₀ mono- or bicyclic aryl-¹⁸F,
l) substituted or unsubstituted O-alkylene-C₅-C₁₀ mono- or bicyclic heteroaryl-¹⁸F or
m) substituted or unsubstituted O—C₃-C₆ cycloalkyl ¹⁸F,
   alkylene being optionally interrupted by one or more O, S or N, with the proviso that one of the substituents R⁸ or R⁹ contains exactly one ¹⁸F isotope and the respective other substituent contains no ¹⁸F isotope,
   L₁ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
   Z₁ represents a metal cation equivalent,
   where n=0, 1, 2 or 3 and
   all diastereomers and enantiomers are included.

Particular preference is furthermore given to each individual compound from the following group, where all possible diastereomers and enantiomers form part of the present subject matter of the invention

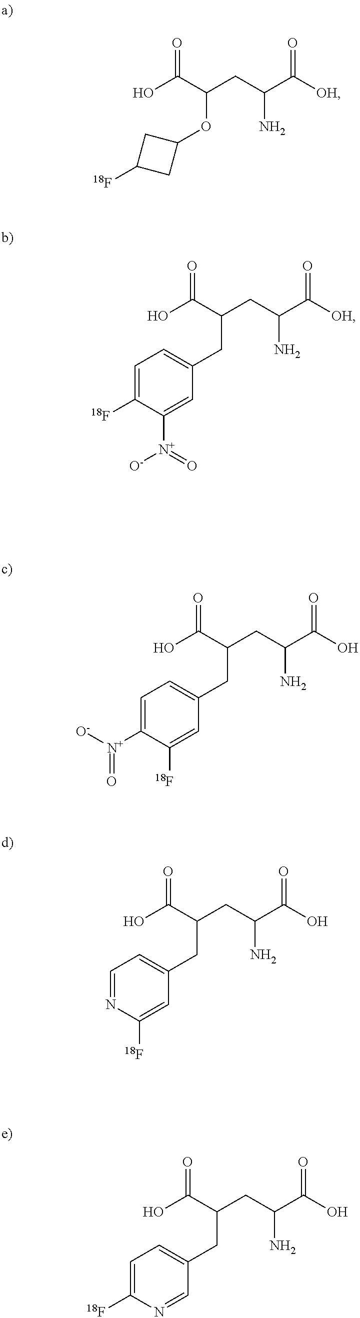

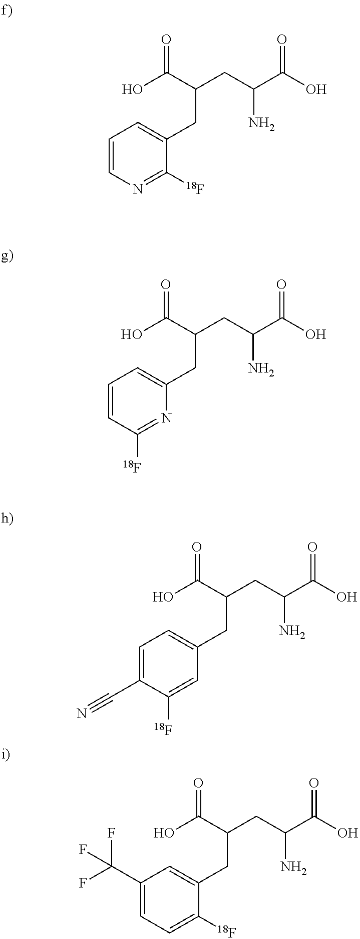

Compounds of the formula V for use as medicaments.

Compounds of the formula V for use for imaging of tumour disorders.

Use of compounds of the formula V for preparing a medicament for imaging of tumour disorders.

Process for preparing compounds of the general formula (V)

which comprises
removing one or more protective groups from a compound of the formula (VI).

According to a further aspect, the present invention relates to compounds of the general formula (VI)

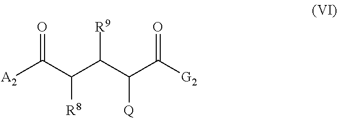

in which
A₂ represents
a) hydroxyl,
b) branched or straight-chain C₁-C₅ alkoxy,
c) branched or straight-chain hydroxy C₁-C₅ alkoxy,
d) branched or straight-chain O—C₁-C₅ alkyl-(O—C₁-C₄ alkyl)ₙ-O—C₁-C₄ alkyl,
e) N(C₁-C₅ alkyl)₂,
f) NH₂,
g) N(H)-L₂ or
h) O-L₂
   G₂ represents
a) hydroxyl,
b) O—Z₂,
c) branched or straight-chain O—C₁-C₅ alkyl,
d) branched or straight-chain O—C₂-C₅ alkenyl,
e) branched or straight-chain O—C₁-C₅ alkyl-(O—C₁-C₄ alkyl)ₙ-O—C₁-C₄ alkyl,
f) branched or straight-chain O—C₁-C₅ alkynyl or
g) triphenylmethoxy,
   R⁸ and R⁹ represent
a) hydrogen,
b) substituted or unsubstituted ¹⁸F—C₆-C₁₀ mono- or bicyclic aryl,
c) substituted or unsubstituted alkylene-C₆-C₁₀-mono- or bicyclic aryl-¹⁸F,
d) substituted or unsubstituted ¹⁸F—C₅-C₁₀ mono- or bicyclic heteroaryl,
e) substituted or unsubstituted alkylene-C₅-C₁₀ mono- or bicyclic heteroaryl-¹⁸F,
f) substituted or unsubstituted ¹⁸F—C₃-C₆ cycloalkyl,
g) substituted or unsubstituted alkylene-C₃-C₆ cycloalkyl-¹⁸F,
h) hydroxyl,
i) branched or straight-chain C₁-C₅ alkyl,
j) branched or straight-chain C₁-C₅ alkoxy,
k) substituted or unsubstituted O-alkylene-C₆-C₁₀ mono- or bicyclic aryl-¹⁸F,
l) substituted or unsubstituted O-alkylene-C₅-C₁₀ mono- or bicyclic heteroaryl-¹⁸F or
m) substituted or unsubstituted O—C₃-C₆ cycloalkyl ¹⁸F,
   alkylene being optionally interrupted by one or more O, S or N,
   with the proviso that exactly one of the substituents R₈ or R⁹ contains exactly one ¹⁸F isotope and the respective other substituent contains no ¹⁸F isotope,
   Q₁ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
e) N(H)-2,2,2-trichloroethoxycarbonyl,
f) N(H)-1,1-dimethylpropynyl,
g) N(H)-1-methyl-1-phenylethoxycarbonyl,
h) N(H)-1-methyl-1-(4-biphenylyl)ethoxycarbonyl,
i) N(H)-cyclobutylcarbonyl,
j) N(H)-1-methylcyclobutylcarbonyl,
k) N(H)-vinylcarbonyl,
l) N(H)-allylcarbonyl,
m) N(H)-adamantylcarbonyl,
n) N(H)-diphenylmethylcarbonyl,
o) N(H)-cinnamylcarbonyl,
p) N(H)-formyl,
q) N(H)-benzoyl,
r) N(H)-trityl,
s) N(H)-p-methoxyphenyldiphenylmethyl,
t) N(H)-di(p-methoxyphenyl)phenylmethyl,
u)

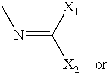

v) N-(tert-butoxycarbonyl)₂,
   L₂ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
   X₁ and X₂ independently of one another represent
a) branched or straight-chain C₁-C₅ alkyl,
b) substituted or unsubstituted aryl,
c) substituted or unsubstituted arylalkyl or
d) substituted or unsubstituted heteroaryl,
   Z₂ represents a metal cation equivalent,
   where n=0, 1, 2 or 3 and
   all diastereomers and enantiomers are included.

Preferred compounds according to the invention of the formula (V and VI) are distinguished in that

R⁸ and R⁹ represent
a) hydrogen
b) substituted or unsubstituted ¹⁸F—C₆-C₁₀ mono- or bicyclic aryl,
c) substituted or unsubstituted ¹⁸F—C₅-C₁₀ mono- or bicyclic heteroaryl,
d) substituted or unsubstituted ¹⁸F—C₃-C₆ cycloalkyl,
e) hydroxyl,
f) branched or straight-chain C₁-C₅ alkyl or
g) branched or straight-chain C₁-C₅ alkoxy.

Preferred compounds of the formula VI are characterized in that R⁸ and R⁹ are selected from the group consisting of hydrogen, substituted or unsubstituted aryl, substituted or unsubstituted ¹⁸F—C₅-C₁₀ heteroaryl and substituted or unsubstituted ¹⁸F—C₃-C₆ cycloalkyl, with the proviso that one of the substituents R⁸ or R⁹ contains exactly one ¹⁸F isotope and the respective other substituent is hydrogen.

Compounds of the formula VI

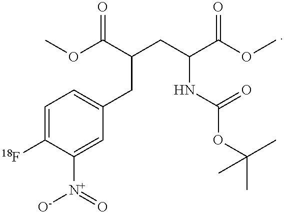

Compounds of the formula VI for use as medicament.

Compounds of the formula VI for use for imaging of tumour disorders.

Use of compounds of the formula VI for preparing a medicament for imaging of tumour disorders.

Process for preparing compounds of the general formula (VI)

which comprises
reacting a compound of the formula (VII) with F-18 fluoride.

According to a further aspect, the present invention relates to compounds of the general formula (VII)

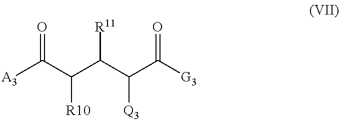

in which
A₃ represents
a) branched or straight-chain C₁-C₅ alkoxy,
b) branched or straight-chain hydroxy C₁-C₅ alkoxy,
c) branched or straight-chain O—C₁-C₅ alkyl-(O—C₁-C₄ alkyl)ₙ-O—C₁-C₄ alkyl,
d) N(C₁-C₅ alkyl)₂,
e) NH₂,
f) N(H)-L₃ or
g) O-L₃,
   G₃ represents
a) O—Z₃,
b) branched or straight-chain O—C₁-C₅ alkyl,
c) branched or straight-chain O—C₂-C₅ alkenyl,
d) branched or straight-chain O—C₁-C₅ alkyl-(O—C₁-C₄ alkyl)ₙ-O—C₁-C₄ alkyl,
e) branched or straight-chain O—C₁-C₅ alkynyl or
f) triphenylmethoxy,
   R¹⁰ and R¹¹ represent
a) hydrogen,
b) substituted or unsubstituted E₁-C₆-C₁₀ mono- or bicyclic aryl,
c) substituted or unsubstituted alkylene-C₆-C₁₀ mono- or bicyclic aryl-E₁,
d) substituted or unsubstituted E₁-C₅-C₁₀ mono- or bicyclic heteroaryl,
e) substituted or unsubstituted alkylene-C₅-C₁₀ mono- or bicyclic heteroaryl-E₁,
f) substituted or unsubstituted E₁-C₃-C₆ cycloalkyl,
g) substituted or unsubstituted alkylene-C₃-C₆-cycloalkyl-E₁,
h) hydroxyl,
i) branched or straight-chain C₁-C₅ alkyl,
j) branched or straight-0chain C₁-C₅ alkoxy,
k) substituted or unsubstituted O-alkylene-C₆-C₁₀ mono- or bicyclic aryl-¹⁸F,
l) substituted or unsubstituted O-alkylene-C₅-C₁₀ mono- or bicyclic heteroaryl-¹⁸F or
m) substituted or unsubstituted O—C₃-C₆ cycloalkyl ¹⁸F,
   alkylene being optionally interrupted by one or more O, S or N, with the proviso that exactly one of the substituents R¹⁰ or R¹¹ contains an E1 and the respective other constituent contains no E₁,
   E₁ represents a leaving group.
   Q₂ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
g) N(H)-2,2,2-trichloroethoxycarbonyl,
h) N(H)-1,1-dimethylpropynyl,
i) N(H)-1-methyl-1-phenylethoxycarbonyl,
j) N(H)-1-methyl-1-(4-biphenylyl)ethoxycarbonyl,
k) N(H)-cyclobutylcarbonyl,
l) N(H)-1-methylcyclobutylcarbonyl,
m) N(H)-vinylcarbonyl,
n) N(H)-allylcarbonyl,
o) N(H)-adamantylcarbonyl,
p) N(H)-diphenylmethylcarbonyl,
q) N(H)-cinnamylcarbonyl,
r) N(H)-formyl,
s) N(H)-benzoyl,
t) N(H)-trityl,
u) N(H)-p-methoxyphenyldiphenylmethyl,
v) N(H)-di(p-methoxyphenyl)phenylmethyl,
w)

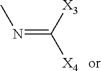

x) N-(tert-butoxycarbonyl)₂,
   L₃ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
   X₃ and X₄ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
   Z₃ represents a metal cation equivalent,
   where n=0, 1, 2 or 3 and
   all diastereomers and enantiomers are included.

Use of compounds of the formula (VII) for preparing compounds of the formula (V) or (VI):

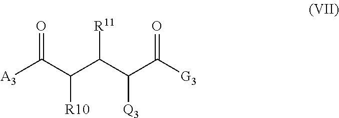

in which
A₃ represents
a) branched or straight-chain C₁-C₅ alkoxy,
b) branched or straight-chain hydroxy C₁-C₅ alkoxy,
c) branched or straight-chain O—C₁-C₅ alkyl-(O—C₁-C₄ alkyl)ₙ-O—C₁-C₄ alkyl,
d) N(C₁-C₅ alkyl)₂,
e) NH₂,
f) N(H)-L₃ or
g) O-L₃,
   G₃ represents
a) O—Z₃,
b) branched or straight-chain O—C₁-C₅ alkyl,
c) branched or straight-chain O—C₂-C₅ alkenyl,
d) branched or straight-chain O—C₁-C₅ alkyl-(O—C₁-C₄ alkyl)ₙ-O—C₁-C₄ alkyl,
e) branched or straight-chain O—C₂-C₅ alkynyl or
f) triphenylmethoxy,
   R¹⁰ and R¹¹ represent
a) hydrogen,
b) substituted or unsubstituted E₁-C₆-C₁₀ mono- or bicyclic aryl,
c) substituted or unsubstituted alkylene-C₆-C₁₀ mono- or bicyclic aryl-E₁,
d) substituted or unsubstituted E₁-C₅-C₁₀ mono- or bicyclic heteroaryl,
e) substituted or unsubstituted alkylene-C₅-C₁₀ mono- or bicyclic heteroaryl-E₁,
f) substituted or unsubstituted E₁-C₃-C₆ cycloalkyl,
g) substituted or unsubstituted alkylene-C₃-C₆ cycloalkyl-E₁,
h) hydroxyl,
i) branched or straight-chain C₁-C₅ alkyl,
j) branched or straight-chain C₁-C₅ alkoxy,
k) substituted or unsubstituted O-alkylene-C₆-C₁₀ mono- or bicyclic aryl-E,
l) substituted or unsubstituted O-alkylene-C₅-C₁₀ mono- or bicyclic heteroaryl-E, or
m) substituted or unsubstituted O—C₃-C₆ cycloalkyl-E,
   alkylene being optionally interrupted by one or more O, S or N,
   with the proviso that exactly one of the substituents R¹⁰ or R¹¹ contains an E₁ and the respective other substituent contains no E₁,
   E₁ represents a leaving group,
   Q₂ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
g) N(H)-2,2,2-trichloroethoxycarbonyl,
h) N(H)-1,1-dimethylpropynyl,
i) N(H)-1-methyl-1-phenylethoxycarbonyl,
j) N(H)-1-methyl-1-(4-biphenylyl)ethoxycarbonyl,
k) N(H)-cyclobutylcarbonyl,
l) N(H)-1-methylcyclobutylcarbonyl,
m) N(H)-vinylcarbonyl,
n) N(H)-allylcarbonyl,
o) N(H)-adamantylcarbonyl,
p) N(H)-diphenylmethylcarbonyl,
q) N(H)-cinnamylcarbonyl,
r) N(H)-formyl,
s) N(H)-benzoyl,
t) N(H)-trityl,
u) N(H)-p-methoxyphenyldiphenylmethyl,
v) N(H)-di(p-methoxyphenyl)phenylmethyl,
w)

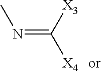

x) N-(tert-butoxycarbonyl)₂,
   L₃ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
   X₃ and X₄ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
   Z₃ represents a metal cation equivalent,
   where n=0, 1, 2 or 3 and
   all diastereomers and enantiomers are included.

Preferred compounds according to the invention of the formula VII are distinguished in that

R¹⁰ and R¹¹ represent
a) hydrogen,
b) substituted or unsubstituted E₁-C₆-C₁₀ mono- or bicyclic aryl,
c) substituted or unsubstituted E₁-C₅-C₁₀ mono- or bicyclic heteroaryl,
d) substituted or unsubstituted E₁-C₃-C₆ cycloaryl,
e) hydroxyl
f) branched or straight-chain C₁-C₅ alkyl or
g) branched or straight-chain C₁-C₅ alkoxy.

According to a further aspect, the present invention relates to compounds of the general formula (VIII)

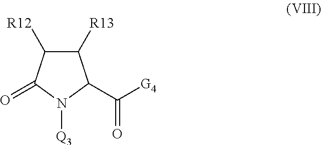

in which
G₄ represents
a) branched or straight-chain O—C₁-C₅ alkyl,
b) branched or straight-chain O—C₂-C₅ alkenyl,
c) branched or straight-chain O—C₁-C₅ alkyl-(O—C₁-C₄ alkyl)ₙ-O—C₁-C₄ alkyl,
d) branched or straight-chain O—C₂-C₅ alkynyl or
e) triphenylmethoxy,
   R¹² and R¹³ represent
a) hydrogen,
b) hydroxyl,
c) branched or straight-chain C₁-C₅ alkyl,
d) branched or straight-chain C₁-C₅ alkoxy or
e) R¹⁴-E₂,
   with the proviso that exactly one of the substituents R¹² or R¹³ contains an E₂ and the respective other substituent contains no E₂,
   E₂ represents a leaving group,
   R¹⁴ represents
a) substituted or unsubstituted C₆-C₁₀ mono- or bicyclic aryl,
b) substituted or unsubstituted alkylene-C₆-C₁₀ mono- or bicyclic aryl,
c) substituted or unsubstituted C₅-C₁₀ mono- or bicyclic heteroaryl,
d) substituted or unsubstituted alkylene-C₅-C₁₀ mono- or bicyclic heteroaryl,
e) substituted or unsubstituted C₃-C₆ cycloalkyl,
f) substituted or unsubstituted alkylene-C₃-C₆ cycloalkyl,
g) substituted or unsubstituted O-alkylene C₅-C₂₀ mono- or bicyclic aryl,
h) substituted or unsubstituted O-alkylene C₅-C₁₀ mono- or bicyclic heteroaryl, or
i) substituted or unsubstituted O—C₃-C₆ cycloalkyl,
   alkylene being optionally interrupted by one or more O, S or N,
   Q₃ represents
a) N-tert-butoxycarbonyl
b) N-allyloxycarbonyl,
c) N-benzyloxycarbonyl,
d) N-ethoxycarbonyl,
e) N-methoxycarbonyl,
f) N-propoxycarbonyl,
g) N-2,2,2-trichloroethoxycarbonyl,
h) hydrogen,
i) N-1-methyl-1-phenylethoxycarbonyl,
j) N-1-methyl-1-(4-biphenylyl)ethoxycarbonyl,
k) N-cyclobutylcarbonyl,
l) N-1-methylcyclobutylcarbonyl,
m) N-vinylcarbonyl,
n) N-allylcarbonyl,
o) N-adamantylcarbonyl,
p) N-diphenylmethylcarbonyl,
q) N-cinnamylcarbonyl,
r) N-formyl
s) N-benzoyl,
t) N(H)-trityl,
u) N(H)-p-methoxyphenyldiphenylmethyl,
v) N(H)-di(p-methoxyphenyl)phenylmethyl,
w)

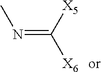

x) N-(tert-butoxycarbonyl)₂,
   X₅ and X₆ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
   where n=0, 1, 2 or 3 and
   all diastereomers and enantiomers are included.

Use of compounds of the formula (VIII) for preparing compounds of the formula (V) or (VI):

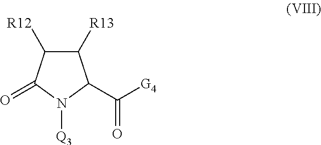

in which
G₄ represents
a) branched or straight-chain O—C₁-C₅ alkyl,
b) branched or straight-chain O—C₂-C₅ alkenyl,
c) branched or straight-chain O—C₁-C₅ alkyl-(O—C₁-C₄ alkyl)ₙ-O—C₁-C₄ alkyl,
d) branched or straight-chain O—C₂-C₅ alkynyl or
e) triphenylmethoxy,
   R¹² and R¹³ represent
a) hydrogen,
b) hydroxyl,
c) branched or straight-chain C₁-C₅ alkyl,
d) branched or straight-chain C₁-C₅ alkoxy or
e) R¹⁴-E₂,
   with the proviso that exactly one of the substituents R¹² or R¹³ contains an E₂ and the respective other substituent contains no E₂,
   E₂ represents a leaving group,
   R₁₄ represents
a) substituted or unsubstituted C₆-C₁₀ mono- or bicyclic aryl,
b) substituted or unsubstituted alkylene-C₆-C₁₀ mono- or bicyclic aryl,
c) substituted or unsubstituted C₅-C₁₀ mono- or bicyclic heteroaryl,
d) substituted or unsubstituted alkylene-C₅-C₁₀ mono- or bicyclic heteroaryl,
e) substituted or unsubstituted C₃-C₆ cycloalkyl,
f) substituted or unsubstituted alkylene-C₃-C₆ cycloalkyl,
g) substituted or unsubstituted O-alkylene C₅-C₁₀ mono- or bicyclic aryl,
h) substituted or unsubstituted O-alkylene C₅-C₁₀ mono- or bicyclic heteroaryl, or
i) substituted or unsubstituted O—C₃-C₆ cycloalkyl,
   alkylene being optionally interrupted by one or more O, S or N,
   Q₃ represents
a) N-tert-butoxycarbonyl
b) N-allyloxycarbonyl,
c) N-benzyloxycarbonyl,
d) N-ethoxycarbonyl,
e) N-methoxycarbonyl,
f) N-propoxycarbonyl,
g) N-2,2,2-trichloroethoxycarbonyl,
h) hydrogen,
i) N-1-methyl-1-phenylethoxycarbonyl,
j) N-1-methyl-1-(4-biphenylyl)ethoxycarbonyl,
k) N-cyclobutylcarbonyl,
l) N-1-methylcyclobutylcarbonyl,
m) N-vinylcarbonyl,
n) N-allylcarbonyl,
o) N-adamantylcarbonyl,
p) N-diphenylmethylcarbonyl,
q) N-cinnamylcarbonyl,
r) N-formyl
s) N-benzoyl,
t) N(H)-trityl,
u) N(H)-p-methoxyphenyldiphenylmethyl,
v) N(H)-di(p-methoxyphenyl)phenylmethyl,
w)

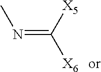

x) N-(tert-butoxycarbonyl)₂,
   X₅ and X₆ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
   where n=0, 1, 2 or 3 and
   all diastereomers and enantiomers are included.

E₁ and E₂ are leaving groups evident or known to the person skilled in the art and mentioned or described, for example, in Synthese (1982), pages 85-125, Table 2, page 86; Carey and Sundberg, Organische Synthese, (1995), pages 279-281, Table 5.8; or Netscher, Recent Res. Dev. Org. Chem., 2003, 7, 71-83, schemes 1, 2, 10 and 15 or in Jerry March, Advanced Organic Chemistry, 4th edition, John Wiley and Sons, pp. 351-56 and 642-653), without being limited thereto.

Preferred compounds according to the invention of the formula (VII or VIII) are distinguished in that

E₁ or E₂ represents
halogen or
sulphonyloxy.

Preferred halogens are iodo, bromo and chloro.

Preferred sulphonyloxy are methanesulphonyloxy, trifluoromethanesulphonyloxy, nonafluorobutyloxy, tosyloxy and nosyloxy.

Preferred compounds according to the invention of the formula (VII or VIII) are distinguished in that

E₁ or E₂ represents
a) chloro,
b) bromo,
c) methanesulphonyloxy,
d) trifluoromethanesulphonyloxy,
e) nonafluorobutyloxy,
f) tosyloxy or
g) iodo.

Preferred compounds according to the invention of the formula VIII are distinguished in that

R₁₄ represents
a) substituted or unsubstituted C₆-C₁₀ mono- or bicyclic aryl,
b) substituted or unsubstituted C₅-C₁₀ mono- or bicyclic heteroaryl,
a) substituted or unsubstituted C₃-C₆ cycloalkyl or,

The invention relates to imaging kits comprising compounds of the general formula VII or VIII.

The invention relates to pharmaceutical compositions comprising compounds of the general formula VI, VI, VII or VIII and suitable pharmaceutical carrier substances.

The invention relates to compounds of the formula I, II, V or VI, characterized in that the compounds are suitable for imaging in a dosage range of 37-600 MBq.

Particularly preferred compounds are characterized in that the compounds are particularly suitable in a dosage range of 150 MBq-370 MBq.

Particularly preferred for introducing the ¹⁸F isotope are

4, 7, 13, 16, 21,24-hexaoxa-1,10-diazabicyclo[8.8.8]-hexacosane K18F (crone ether salt Kryptofix K18F),

### K¹⁸F,

### H¹⁸F,

### KH¹⁸F₂,

### Cs¹⁸F,

### Na¹⁸F or

¹⁸F tetraalkylammonium salt (for example [F-18]tetra-butylammonium fluoride).

If a compound of the formula (I) to formula (VIII) of the present subject matter of the invention contains one or more centres of chirality, the present invention embraces all forms of this isomer including both enantiomers and all possible diastereomers. Compounds containing at least one centre of chirality can be used as a racemic mixture, if appropriate as a diastereomer mixture or a diastereomerically enriched mixture or else an enantiomerically enriched mixture. The racemic enantiomerically enriched mixture or diastereomer mixture may, if appropriate, be separated by methods known to the person skilled in the art, so that the enantiomers or diastereomers can be used individually. In cases where a carbon-carbon double bond is present, both the “cis” and “trans” isomer form part of the present invention. In cases where tautomeric forms may be present, such as, for example, keto-enol tautomerism, the present invention embraces all tautomeric forms, but these forms may be present in equilibrium or, preferably, in one form.

The compounds of the general formulae I to VIII and their preferred embodiments are used as medicaments.

The compounds of the general formula I, II, V or VI according to the invention and their preferred embodiments are used in the diagnosis of physiological or pathological conditions.

These compounds are preferably used in the non-invasive PET-based diagnosis on the human or animal body.

Particularly preferably, the compounds of the general formula I, II, V or VI according to the invention and their preferred embodiments are used in the diagnosis of tumour disorders. Examples of such tumour disorders are malignomas of the gastrointestinal or colorectal tract, liver carcinoma, pancreas carcinoma, kidney carcinoma, bladder carcinoma, thyroid carcinoma, prostrate carcinoma, endometrial carcinoma, ovary carcinoma, testes carcinoma, melanoma, small-cell and non-small-cell bronchial carcinoma, dysplastic oral mucosa carcinoma, invasive oral cancer; breast cancer, including hormone-dependent and hormone-independent breast cancer, squamous cell carcinoma, neurological cancer disorders including neuroblastoma, glioma, astrocytoma, osteosarcoma, meningioma, soft tissue sarcoma; haemangioma and endocrine tumours, including pituitary adenoma, chromocytoma, paraganglioma, haematological tumour disorders including lymphoma and leukaemias; or metastases of one of the tumours mentioned above.

The compounds of the general formula I, II, V or VI according to the invention and their preferred embodiments are used for preparing a medicament for the diagnosis of tumour disorders. Examples of such tumour disorders are malignomas of the gastrointestinal or colorectal tract, liver carcinoma, pancreas carcinoma, kidney carcinoma, bladder carcinoma, thyroid carcinoma, prostrate carcinoma, endometrial carcinoma, ovary carcinoma, testes carcinoma, melanoma, small-cell and non-small-cell bronchial carcinoma, dysplastic oral mucosa carcinoma, invasive oral cancer; breast cancer, including hormone-dependent and hormone-independent breast cancer, squamous cell carcinoma, neurological cancer disorders including neuroblastoma, glioma, astrocytoma, osteosarcoma, meningioma, soft tissue sarcoma; haemangioma and endocrine tumours, including pituitary adenoma, chromocytoma, paraganglioma, haematological tumour disorders including lymphoma and leukaemias; or metastases of one of the tumours mentioned above.

The invention relates to pharmaceutical preparations comprising at least one compound of the formula I or II and also a pharmaceutically acceptable carrier.

To the use of the compounds of the formula I or II as medicaments, they are brought into the form of a pharmaceutical preparation which, in addition to the active compound, comprises pharmaceutical organic or inorganic inert carrier materials suitable for enteral or parenteral administration, such as, for example, water, gelatine, gum Arabic, lactose, starch, magnesium stearate, talcum, vegetable oils, polyalkylene glycols, etc.

The invention relates to a kit comprising at least one compound of the formula I to VIII.

At physiological pH 7.4, the compounds according to the invention may also be present as zwitterions or salts, as is known to those skilled in the art.

The invention relates to
1) Compounds of the general formula I

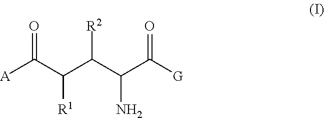

in which
A represents
a) hydroxyl,
b) branched or straight-chain C₁-C₅ alkoxy,
c) branched or straight-chain hydroxy C₁-C₅ alkoxy,
d) branched or straight-chain O—C₁-C₅ alkyl-(O—C₁-C₄ alkyl)ₙ-O—C₁-C₄ alkyl,
e) N(C₁-C₅ alkyl)₂,
f) NH₂,
g) N(H)-L,
h) O-L or
i) O—Z,
   G represents
a) hydroxyl,
b) O—Z,
c) branched or straight-chain O—C₁-C₅ alkyl,
d) branched or straight-chain O—C₂-C₅ alkenyl,
e) branched or straight-chain O—C₁-C₅ alkyl-(O—C₁-C₄ alkyl)ₙ-O—C₁-C₄ alkyl,
f) branched or straight-chain O—C₁-C₅ alkynyl or
g) triphenylmethoxy,
   R¹ and R² represent
i) hydrogen,
j) branched or straight-chain ¹⁸F—C₆-C₁₀ alkoxy,
k) branched or straight-chain ¹⁸F—C₆-C₁₀ alkyl,
l) branched or straight-chain ¹⁸F—C₆-C₁₀ alkenyl,
m) branched or straight-chain ¹⁸F—C₆-C₁₀ alkynyl,
n) hydroxyl,
o) branched or straight-chain C₁-C₅ alkyl or
p) branched or straight-chain C₁-C₅ alkoxy,
with the proviso that one of the substituents R² or R² contains exactly one ¹⁸F isotope and the respective other substituent contains no ¹⁸F isotope,
   L represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₁-C₅ alkynyl, and
   Z represents a metal cation equivalent,
   where n=0, 1, 2 or 3 and
   all diastereomers and enantiomers are included.
2) Compounds according to claim 1, characterized in that A represents hydroxyl, branched or straight-chain C₁-C₅ alkoxy or NH₂.
3) Compounds according to claim 1 or 2, characterized in that A represents NH₂.
4) Compounds according to any of claims 1 to 3, characterized in that R¹ and R² are selected from the group consisting of hydrogen, ¹⁸F-hexoxy, ¹⁸F-heptoxy, ¹⁸F-octoxy, ¹⁸F-nonoxy, ¹⁸F-decoxy, ¹⁸F-hexyl, ¹⁸F-heptyl, ¹⁸F-octyl, ¹⁸F-nonyl and ¹⁸F-decyl, with the proviso that one of the substituents R¹ or R² contains exactly one ¹⁸F isotope and the respective other substituent is hydrogen.
5) Compounds according to any of claims 1 to 4, characterized in that G is selected from the group consisting of hydroxyl and branched or straight-chain O—C₁-C₄ alkyl.
6) Compounds according to any of claims 1 to 5, characterized in that G is methoxy.
7) Compounds according to any of claims 1 to 6, characterized in that Z is selected from the group consisting of Mg²⁺, Ca²⁺, Na⁺ and K⁺.
8) Compounds according to claim 1, selected from the group of compounds of the formulae:

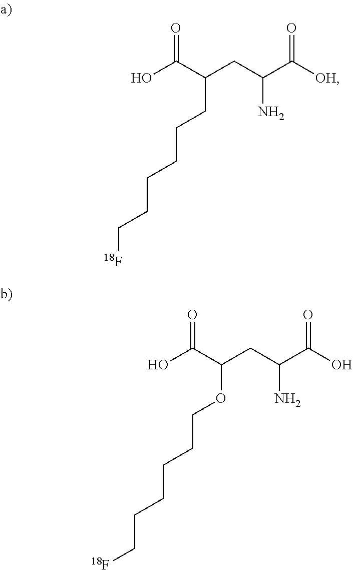

9) Compounds according to any of claims 1 to 8 for use as medicaments.
10) Compounds according to any of claims 1 to 8 for use for imaging in tumour disorders.
11) Use of compounds according to any of claims 1 to 8 for preparing a medicament for imaging in tumour disorders.
12) Process for preparing compounds of the general formula (I) according to any of claims 1 to 8, which comprises
   removing one or more protective groups pre-sent in a compound of the formula (II) according to any of claims 13 to 22.
13) Compounds of the general formula (II):

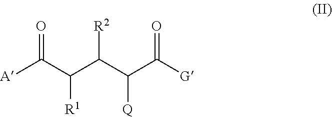

which
A′ represents
   a) hydroxyl,
   b) branched or straight-chain C₁-C₅ alkoxy,
   c) branched or straight-chain hydroxy C₁-C₅ alkoxy,
   d) branched or straight-chain O—C₁-C₅ alkyl-(O—C₁-C₄ alkyl)ₙ-O—C₁-C₄ alkyl,
   e) N(C₁-C₅ alkyl)₂,
   f) NH₂,
   g) N(H)-L′,
   h) O-L′,
G′ represents
   a) hydroxyl,
   b) O—Z′,
   c) branched or straight-chain O—C₁-C₅ alkyl,
   d) branched or straight-chain O—C₂-C₅ alkenyl,
   e) branched or straight-chain O—C₁-C₅ alkyl-(O—C₁-C₄ alkyl)ₙ-O—C₁-C₄ alkyl,
   f) branched or straight-chain O—C₂-C₅ alkynyl or
   g) triphenylmethoxy,
R¹ and R² represent
   a) hydrogen,
   b) branched or straight-chain ¹⁸F—C₆-C₁₀ alkoxy,
   c) branched or straight-chain ¹⁸F—C₆-C₁₀ alkyl,
   d) branched or straight-chain ¹⁸F—C₆-C₁₀ alkenyl,
   e) branched or straight-chain ¹⁸F—C₆-C₁₀ alkynyl,
   f) hydroxyl,
   g) branched or straight-chain C₁-C₅ alkyl or
   h) branched or straight-chain C₁-C₅ alkoxy,
   with the proviso that exactly one of the substituents R¹ or R² contains exactly one ¹⁸F isotope and the respective other substituent contains no ¹⁸F isotope,
Q represents
   a) N(H)-tert-butoxycarbonyl,
   b) N(H)-allyloxycarbonyl,
   c) N(H)-benzyloxycarbonyl,
   d) N(H)-ethoxycarbonyl,
   e) N(H)-methoxycarbonyl,
   f) N(H)-propoxycarbonyl,
   e) N(H)-2,2,2-trichloroethoxycarbonyl,
   f) N(H)-1,1-dimethylpropynyl,
   g) N(H)-1-methyl-1-phenylethoxycarbonyl,
   h) N(H)-1-methyl-1-(4-biphenylyl)ethoxycarbonyl,
   i) N(H)-cyclobutylcarbonyl,
   j) N(H)-1-methylcyclobutylcarbonyl,
   k) N(H)-vinylcarbonyl,
   l) N(H)-allylcarbonyl,
   m) N(H)-adamantylcarbonyl,
   n) N(H)-diphenylmethylcarbonyl,
   o) N(H)-cinnamylcarbonyl,
   p) N(H)-formyl,
   q) N(H)-benzoyl,
   r) N(H)-trityl,
   s) N(H)-p-methoxydiphenylmethyl,
   t) N(H)-di(p-methoxyphenyl)phenylmethyl,
   u)

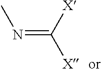

v) N-(tert-butoxycarbonyl)₂,
L′ represents
   a) branched or straight-chain C₁-C₅ alkyl,
   b) branched or straight-chain C₂-C₅ alkenyl,
   c) branched or straight-chain C₁-C₅ alkyl-(O—C₂-C₄ alkyl)ₙ-O—C₁-C₄ alkyl or
   d) branched or straight-chain C₂-C₅ alkynyl,
X′ and X″ independently of one another represent
   a) branched or straight-chain C₁-C₅ alkyl,
   b) substituted or unsubstituted aryl,
   c) substituted or unsubstituted aralkyl or
   d) substituted or unsubstituted heteroaryl,
Z′ represents a metal cation equivalent,
where n=0, 1, 2 or 3 and
all diastereomers and enantiomers are included.
14) Compounds according to claim 13, characterized in that A′ represents hydroxyl, branched or straight-chain C₁-C₅ alkoxy or NH₂.
15) Compounds according to claim 14, characterized in that A′ represents NH₂.
16) Compounds according to any of claims 13 to 15, characterized in that R¹ and R² are selected from the group consisting of hydrogen, ¹⁸F-hexoxy, ¹⁸F-heptoxy, ¹⁸F-octoxy, ¹⁸F-nonoxy, ¹⁸F-decoxy, ¹⁸F-hexyl, ¹⁸F-heptyl, ¹⁸F-octyl, ¹⁸F-nonyl and ¹⁸F-decyl, with the proviso that one of the substituents R¹ or R² contains exactly one ¹⁸F isotope and the respective other substituent is hydrogen.
17) Compounds according to any of claims 13 to 16, characterized in that G′ is selected from the group consisting of hydroxyl, branched or straight-chain C₁-C₄ alkoxy and OZ′.
18) Compounds according to any of claims 13 to 17, characterized in that G′ represents methoxy.
19) Compounds according to any of claims 13 to 18, characterized in that Z′ is selected from the group consisting of Na⁺, K⁺, Ca²⁺ and Mg²⁺.
20) Compounds according to any of claims 13 to 19, characterized in that Q is selected from the group consisting of N(H)-tert-butoxycarbonyl, N(H)-benzyloxycarbonyl and

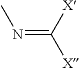

in which
X′ and X″ independently of one another represent
   a) branched or straight-chain C₁-C₅ alkyl,
   b) substituted or unsubstituted aryl,
   c) substituted or unsubstituted aralkyl or
   d) substituted or unsubstituted heteroaryl.
21) Compounds according to any of claims 13 to 20, characterized in that Q represents N(H)-tert-butoxycarbonyl or N(H)-trityl.
22) Compounds according to claim 13, selected from the group of compounds of the formulae:

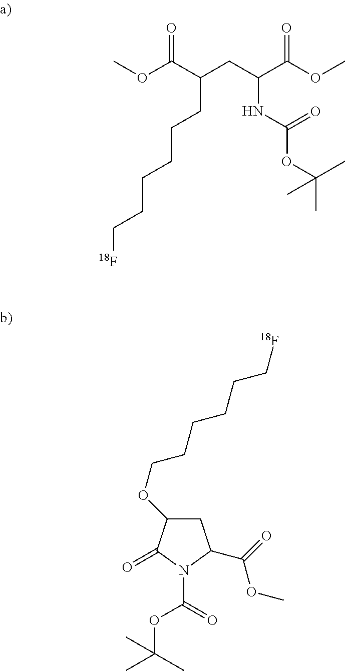

23) Compounds according to any of claims 13 to 22 for use as medicaments.
24) Compounds according to any of claims 13 to 22 for use for imaging in tumour disorders.
25) Use of compounds according to any of claims 1 to 22 for preparing a medicament for imaging in tumour disorders.
26) Process for preparing compounds of the general formula (II) according to any of claims 13 to 22, which comprises
   reacting a compound of the formula (III) according to claim 27 with F-18 fluoride.
27) Compounds of the formula (III)

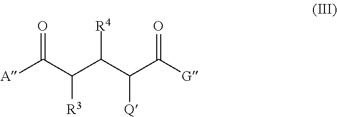

in which
A″ represents
   a) branched or straight-chain C₁-C₅ alkoxy,
   b) branched or straight-chain hydroxy C₁-C₅ alkoxy,
   c) branched or straight-chain O—C₁-C₅ alkyl-(O—C₁-C₄ alkyl)ₙ-O—C₁-C₄ alkyl,
   d) N(C₁-C₅ alkyl)₂,
   e) NH₂,
   f) N(H)-L″,
   g) O-L″,
G″ represents
   a) branched or straight-chain O—C₁-C₅ alkyl,
   b) branched or straight-chain O—C₂-C₅ alkenyl,
   c) branched or straight-chain O—C₁-C₅ alkyl-(O—C₁-C₄ alkyl)ₙ-O—C₁-C₄ alkyl,
   d) branched or straight-chain O—C₁-C₅ alkynyl or
   e) triphenylmethoxy,
R³ and R⁴ represent
   a) hydrogen,
   b) branched or straight-chain E-C₆-C₁₀ alkoxy,
   c) branched or straight-chain E-C₆-C₁₀ alkyl,
   d) branched or straight-chain E-C₆-C₁₀ alkenyl,
   e) branched or straight-chain E-C₆-C₁₀ alkynyl,
   f) hydroxyl,
   g) branched or straight-chain C₁-C₅ alkyl or
   h) branched or straight-chain C₁-C₅ alkoxy,
   with the proviso that exactly one of the substituents R³ or R⁴ contains an E and the respective other substituent contains no E,
E represents
   a) chloro,
   b) bromo,
   c) mesyloxy,
   d) trifluoromethylsulphonyloxy,
   e) nonafluorobutyloxy,
   f) tosyloxy or
   g) iodo,
Q′ represents
   a) N(H)-tert-butoxycarbonyl
   b) N(H)-allyloxycarbonyl,
   c) N(H)-benzyloxycarbonyl,
   d) N(H)-ethoxycarbonyl,
   e) N(H)-methoxycarbonyl,
   f) N(H)-propoxycarbonyl,
   g) N(H)-2,2,2-trichloroethoxycarbonyl,
   h) N(H)-1,1-dimethylpropynyl,
   i) N(H)-1-methyl-1-phenylethoxycarbonyl,
   j) N(H)-1-methyl-1-(4-biphenylyl)ethoxycarbonyl,
   k) N(H)-cyclobutylcarbonyl,
   l) N(H)-1-methylcyclobutylcarbonyl,
   m) N(H)-vinylcarbonyl,
   n) N(H)-allylcarbonyl,
   o) N(H)-adamantylcarbonyl,
   p) N(H)-diphenylmethylcarbonyl,
   q) N(H)-cinnamylcarbonyl,
   r) N(H)-formyl,
   s) N(H)-benzoyl,
   t) N(H)-trityl,
   u) N(H)-p-methoxydiphenylmethyl,
   v) N(H)-di(p-methoxyphenyl)phenylmethyl,
   w)

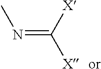

x) N-(tert-butoxycarbonyl)₂,
L″ represents
   a) branched or straight-chain C₁-C₅ alkyl,
   b) branched or straight-chain C₂-C₅ alkenyl,
   c) branched or straight-chain C₁-C₅ alkyl-(O—C₂-C₄ alkyl)ₙ-O—C₁-C₄ alkyl or
   d) branched or straight-chain C₁-C₅ alkynyl,
X′ and X″ independently of one another represent
   a) branched or straight-chain C₁-C₅ alkyl,
   b) substituted or unsubstituted aryl,
   c) substituted or unsubstituted alkylaryl or
   d) substituted or unsubstituted heteroaryl,
where n=0, 1, 2 or 3 and
all diastereomers and enantiomers are included.
28) Use of compounds of the formula (III) for preparing compounds of the formula (I) or (II):

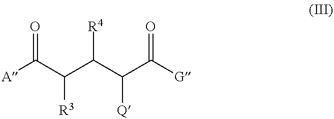

in which
A″ represents
   a) branched or straight-chain C₁-C₅ alkoxy,
   b) branched or straight-chain hydroxy C₁-C₅ alkoxy,
   c) branched or straight-chain O—C₁-C₅ alkyl-(O—C₁-C₄ alkyl)ₙ-O—C₁-C₄ alkyl,
   d) N(C₁-C₅ alkyl)₂,
   e) NH₂,
   f) N(H)-L″,
   g) O-L″,
G″ represents
   a) branched or straight-chain O—C₁-C₅ alkyl,
   b) branched or straight-chain O—C₂-C₅ alkenyl,
   c) branched or straight-chain O—C₁-C₅ alkyl-(O—C₁-C₄ alkyl)ₙ-O—C₁-C₄ alkyl,
   d) branched or straight-chain O—C₂-C₅ alkynyl or
   e) triphenylmethoxy,
R³ and R⁴ represent
   a) hydrogen,
   b) branched or straight-chain E-C₆-C₁₀ alkoxy,
   c) branched or straight-chain E-C₆-C₁₀ alkyl,
   d) branched or straight-chain E-C₆-C₁₀ alkenyl,
   e) branched or straight-chain E-C₆-C₁₀ alkynyl,
   f) hydroxyl,
   g) branched or straight-chain C₁-C₅ alkyl or
   h) branched or straight-chain C₁-C₅ alkoxy,
   with the proviso that exactly one of the substituents R³ or R⁴ contains an E and the respective other substituent contains no E,
E represents
   a) chloro,
   b) bromo,
   c) methanesulphonyloxy,
   d) trifluoromethanesulphonyloxy,
   e) nonafluorobutyloxy,
   f) tosyloxy or
   g) iodo,
Q′ represents
   a) N(H)-tert-butoxycarbonyl
   b) N(H)-allyloxycarbonyl,
   c) N(H)-benzyloxycarbonyl,
   d) N(H)-ethoxycarbonyl,
   e) N(H)-methoxycarbonyl,
   f) N(H)-propoxycarbonyl,
   g) N(H)-2,2,2-trichloroethoxycarbonyl,
   h) N(H)-1,1-dimethylpropynyl,
   i) N(H)-1-methyl-1-phenylethoxycarbonyl,
   j) N(H)-1-methyl-1-(4-biphenylyl)ethoxycarbonyl,
   k) N(H)-cyclobutylcarbonyl,
   l) N(H)-1-methylcyclobutylcarbonyl,
   m) N(H)-vinylcarbonyl,
   n) N(H)-allylcarbonyl,
   o) N(H)-adamantylcarbonyl,
   p) N(H)-diphenylmethylcarbonyl,
   q) N(H)-cinnamylcarbonyl,
   r) N(H)-formyl,
   s) N(H)-benzoyl,
   t) N(H)-trityl,
   u) N(H)-p-methoxyphenyldiphenylmethyl,
   v) N(H)-di(p-methoxyphenyl)phenylmethyl,
   w)

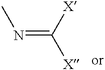

x) N-(tert-butoxycarbonyl)₂,
L″ represents
   a) branched or straight-chain C₁-C₅ alkyl,
   b) branched or straight-chain C₂-C₅ alkenyl,
   c) branched or straight-chain C₁-C₅ alkyl-(O—C₂-C₄ alkyl)ₙ-O—C₁-C₄ alkyl or
   d) branched or straight-chain C₁-C₅ alkynyl,
X′ and X″ independently of one another represent
   a) branched or straight-chain C₁-C₅ alkyl,
   b) substituted or unsubstituted aryl,
   c) substituted or unsubstituted alkylaryl or
   d) substituted or unsubstituted heteroaryl,
where n=0, 1, 2 or 3 and
all diastereomers and enantiomers are included.
29) Compounds of the formula (IV)

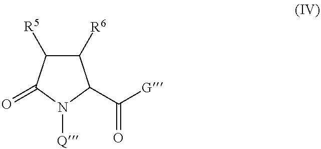

in which
G′″ represents
   a) branched or straight-chain O—C₁-C₅ alkyl,
   b) branched or straight-chain O—C₂-C₅ alkenyl,
   c) branched or straight-chain O—C₁-C₅ alkyl-(O—C₁-C₄ alkyl)ₙ-O—C₁-C₄ alkyl,
   d) branched or straight-chain O—C₁-C₅ alkynyl or
   e) triphenylmethoxy,
R⁵ and R⁶ represent
   a) hydrogen,
   b) hydroxyl,
   c) branched or straight-chain C₁-C₅ alkyl,
   d) branched or straight-chain C₁-C₅ alkoxy or
   e) R⁷-E′,
   with the proviso that exactly one of the substituents R⁵ or R⁶ contains an E′ and the respective other substituent contains no E′,
E′ represents
   a) chloro,
b) bromo,
c) methanesulphonyloxy,
d) trifluoromethanesulphonyloxy,
e) nonafluorobutyloxy,
f) tosyloxy or
   g) iodo,
R⁷ represents
   a) branched or straight-chain C₆-C₁₀ alkoxy,
   b) branched or straight-chain C₆-C₁₀ alkyl,
   c) branched or straight-chain C₆-C₁₀ alkenyl or
   d) branched or straight-chain C₆-C₁₀ alkynyl,
Q′″ represents
   a) N-tert-butoxycarbonyl
   b) N-allyloxycarbonyl,
   c) N-benzyloxycarbonyl,
   d) N-ethoxycarbonyl,
   e) N-methoxycarbonyl,
   f) N-propoxycarbonyl,
   g) N-2,2,2-trichloroethoxycarbonyl,
   h) hydrogen,
   i) N-1-methyl-1-phenylethoxycarbonyl,
   j) N-1-methyl-1-(4-biphenylyl)ethoxycarbonyl,
   k) N-cyclobutylcarbonyl,
   l) N-1-methylcyclobutylcarbonyl,
   m) N-vinylcarbonyl,
   n) N-allylcarbonyl,
   o) N-adamantylcarbonyl,
   p) N-diphenylmethylcarbonyl,
   q) N-cinnamylcarbonyl,
   r) N-formyl,
   s) N-benzoyl,
   t) N(H)-trityl,
   u) N(H)-p-methoxyphenyldiphenylmethyl,
   v) N(H)-di(p-methoxyphenyl)phenylmethyl,
   w)

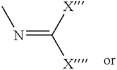

x) N-(tert-butoxycarbonyl)₂,
X′″ and X″″ independently of one another represent
   a) branched or straight-chain C₁-C₅ alkyl,
   b) substituted or unsubstituted aryl,
   c) substituted or unsubstituted alkylaryl or
   d) substituted or unsubstituted heteroaryl,
where n=0, 1, 2 or 3 and
all diastereomers and enantiomers are included.
30) Use of compounds of the formula (VI) for preparing compounds of the formula (I) or (II):

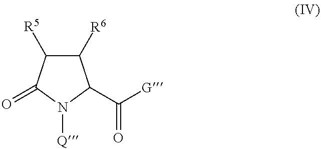

in which
G′″ represents
   a) branched or straight-chain O—C₁-C₅ alkyl,
   b) branched or straight-chain O—C₂-C₅ alkenyl,
   c) branched or straight-chain O—C₁-C₅ alkyl-(O—C₁-C₄ alkyl)ₙ-O—C₁-C₄ alkyl,
   d) branched or straight-chain O—C₂-C₅ alkynyl or
   e) triphenylmethoxy,
R⁵ and R⁶ represent
   a) hydrogen,
   b) hydroxyl,
   c) branched or straight-chain C₁-C₅ alkyl,
   d) branched or straight-chain C₁-C₅ alkoxy or
   e) R⁷-E′,
   with the proviso that exactly one of the substituents R⁵ or R⁶ contains an E′ and the respective other substituent contains no E′,
E′ represents
   a) chloro,
   b) bromo,
   c) methanesulphonyloxy,
   d) trifluoromethanesulphonyloxy,
   e) nonafluorobutyloxy,
   f) tosyloxy or
   g) iodo,
R⁷ represents
   a) branched or straight-chain C₆-C₁₀ alkoxy,
   b) branched or straight-chain C₆-C₁₀ alkyl,
   c) branched or straight-chain C₆-C₁₀ alkenyl or
   d) branched or straight-chain C₆-C₁₀ alkynyl,
Q′″ represents
   a) N-tert-butoxycarbonyl
   b) N-allyloxycarbonyl,
   c) N-benzyloxycarbonyl,
   d) N-ethoxycarbonyl,
   e) N-methoxycarbonyl,
   f) N-propoxycarbonyl,
   g) N-2,2,2-trichloroethoxycarbonyl,
   h) hydrogen,
   i) N-1-methyl-1-phenylethoxycarbonyl,
   j) N-1-methyl-1-(4-biphenylyl)ethoxycarbonyl,
   k) N-cyclobutylcarbonyl,
   l) N-1-methylcyclobutylcarbonyl,
   m) N-vinylcarbonyl,
   n) N-allylcarbonyl,
   o) N-adamantylcarbonyl,
   p) N-diphenylmethylcarbonyl,
   q) N-cinnamylcarbonyl,
   r) N-formyl,
   s) N-benzoyl,
   t) N(H)-trityl,
   u) N(H)-p-methoxyphenyldiphenylmethyl,
   v) N(H)-di(p-methoxyphenyl)phenylmethyl,
   w)

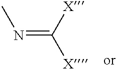

x) N-(tert-butoxycarbonyl)₂,
X′″ and X″″ independently of one another represent
   a) branched or straight-chain C₁-C₅ alkyl,
   b) substituted or unsubstituted aryl,
   c) substituted or unsubstituted alkylaryl or
   d) substituted or unsubstituted heteroaryl,
where n=0, 1, 2 or 3 and
all diastereomers and enantiomers are included.
31) Imaging kit, comprising compounds of the general formula III or IV.
32) Pharmaceutical composition, comprising compounds of the general formula I, II, III or IV and suitable pharmaceutical carrier substances.
33) Compounds of the general formula V

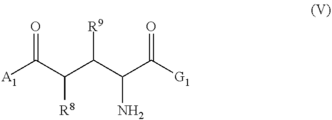

in which
A₁ represents
   a) hydroxyl,
   b) branched or straight-chain C₁-C₅ alkoxy,
   c) branched or straight-chain hydroxy C₁-C₅ alkoxy,
   d) branched or straight-chain O—C₁-C₅ alkyl-(O—C₁-C₄ alkyl)_{μ}—O—C₁-C₄ alkyl,
   ed) N(C₁-C₅ alkyl)₂,
   f) NH₂,
   g) N(H)-L₁,
   h) O-L₁ or
   i) O—Z₁,
G₁ represents
   a) hydroxyl,
   b) O—Z₁,
   c) branched or straight-chain O—C₁-C₅ alkyl,
   d) branched or straight-chain O—C₂-C₅ alkenyl,
   e) branched or straight-chain O—C₁-C₅ alkyl-(O—C₁-C₄ alkyl)ₙ-O—C₁-C₄ alkyl,
   f) branched or straight-chain O—C₂-C₅ alkynyl or
   g) triphenylmethoxy,
R⁸ and R⁹ represent
   a) hydrogen,
   b) substituted or unsubstituted ¹⁸F—C₆-C₁₀ mono- or bicyclic aryl,
   c) substituted or unsubstituted ¹⁸F—C₅-C₁₀ mono- or bicyclic heteroaryl,
   d) substituted or unsubstituted ¹⁸F—C₃-C₆ cycloalkyl,
   e) hydroxyl,
   f) branched or straight-chain C₁-C₅ alkyl or
   g) branched or straight-chain C₁-C₅ alkoxy,
   with the proviso that one of the substituents R⁸ or R⁹ contains exactly one ¹⁸F isotope and the respective other substituent contains no ¹⁸F isotope,
L₁ represents
   a) branched or straight-chain C₁-C₅ alkyl,
   b) branched or straight-chain C₂-C₅ alkenyl,
   c) branched or straight-chain C₁-C₅ alkyl-(O—C₁-C₄ alkyl)ₙ-O—C₁-C₄ alkyl or
   d) branched or straight-chain C₁-C₅ alkynyl,
Z₁ represents a metal cation equivalent,
where n=0, 1, 2 or 3 and
all diastereomers and enantiomers are included.
34) Compounds according to claim 33, characterized in that A₁ represents hydroxyl, branched or straight-chain C₁-C₅ alkoxy or NH₂.
35) Compounds according to claim 33 or 34, characterized in that A₁ represents NH₂.
36) Compounds according to any of claims 33 to 35, characterized in that R⁸ and R⁹ are selected from the group consisting of hydrogen, substituted or unsubstituted ¹⁸F—C₆-C₁₀ aryl, substituted or unsubstituted ¹⁸F—C₅-C₁₀ heteroaryl, and substituted or unsubstituted ¹⁸F—C₃-C₆ cycloalkyl, with the proviso that one of the substituents R⁸ or R⁹ contains exactly one ¹⁸F isotope and the respective other substituent is hydrogen.
37) Compounds according to any of claims 33 to 36, characterized in that G₁ is selected from the group consisting of hydroxyl and branched or straight-chain O—C₁-C₄ alkyl.
38) Compounds according to claim 37, characterized in that G₁ is methoxy.
39) Compounds according to any of claims 33 to 38, characterized in that Z₁ is selected from the group consisting of Mg²⁺, Ca²⁺, Na⁺ and K⁺.
40) Compounds according to claim 33, selected from the group of compounds of the formulae:

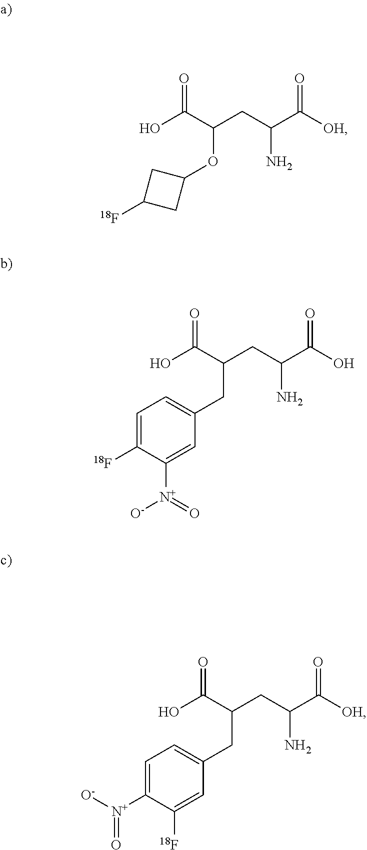

41) Compounds according to any of claims 33 to 40 for use as medicaments.
42) Compounds according to any of claims 33 to 40 for use for imaging in tumour disorders.
43) Use of compounds according to any of claims 33 to 40 for preparing a medicament for imaging in tumour disorders.
44) Process for preparing compounds of the general formula (V) according to any of claims 33 to 40, which comprises
   removing one or more protective groups pre-sent in a compound of the formula (VI) according to any of claims 45 to 54.
45) Compounds of the general formula (VI):

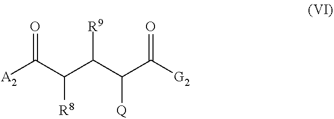

in which
A₂ represents
   a) hydroxyl,
   b) branched or straight-chain C₁-C₅ alkoxy,
   c) branched or straight-chain hydroxy C₁-C₅
   alkoxy,
   d) branched or straight-chain O—C₁-C₅ alkyl-(O—C₁-C₄ alkyl)ₙ-O—C₁-C₄ alkyl,
   e) N(C₁-C₅ alkyl)₂,
   f) NH₂,
   g) N(H)-L₂ or
   h) O-L₂
G₂ represents
   a) hydroxyl,
   b) O—Z₂,
   c) branched or straight-chain O—C₁-C₅ alkyl,
   d) branched or straight-chain O—C₂-C₅ alkenyl,
   e) branched or straight-chain O—C₁-C₅ alkyl-(O—C₁-C₄ alkyl)ₙ-O—C₁-C₄ alkyl,
   f) branched or straight-chain O—C₂-C₅ alkynyl or
   g) triphenylmethoxy,
R⁸ and R⁹ represent
   a) hydrogen,
   b) substituted or unsubstituted ¹⁸F—C₆-C₁₀ mono- or bicyclic aryl,
   c) substituted or unsubstituted ¹⁸F—C₅-C₁₀ mono- or bicyclic heteroaryl,
   d) substituted or unsubstituted ¹⁸F—C₃-C₆ cycloalkyl,
   e) hydroxyl,
   f) branched or straight-chain C₁-C₅ alkyl or
   g) branched or straight-chain C₁-C₅ alkoxy,
   with the proviso that exactly one of the substituents R⁸ or R⁹ contains exactly one ¹⁸F isotope and the respective other substituent contains no ¹⁸F isotope,
Q₁ represents
   a) N(H)-tert-butoxycarbonyl
   b) N(H)-allyloxycarbonyl,
   c) N(H)-benzyloxycarbonyl,
   d) N(H)-ethoxycarbonyl,
   e) N(H)-methoxycarbonyl,
   f) N(H)-propoxycarbonyl,
   e) N(H)-2,2,2-trichloroethoxycarbonyl,
   f) N(H)-1,1-dimethylpropynyl,
   g) N(H)-1-methyl-1-phenylethoxycarbonyl,
   h) N(H)-1-methyl-1-(4-biphenylyl)ethoxycarbonyl,
   i) N(H)-cyclobutylcarbonyl,
   j) N(H)-1-methylcyclobutylcarbonyl,
   k) N(H)-vinylcarbonyl,
   l) N(H)-allylcarbonyl,
   m) N(H)-adamantylcarbonyl,
   n) N(H)-diphenylmethylcarbonyl,
   o) N(H)-cinnamylcarbonyl,
   p) N(H)-formyl,
   q) N(H)-benzoyl,
   r) N(H)-trityl,
   s) N(H)-p-methoxyphenyldiphenylmethyl,
   t) N(H)-di(p-methoxyphenyl)phenylmethyl,
   u)

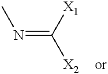

v) N-(tert-butoxycarbonyl)₂,
L₂ represents
   a) branched or straight-chain C₁-C₅ alkyl,
   b) branched or straight-chain C₂-C₅ alkenyl,
   c) branched or straight-chain C₁-C₅ alkyl-(O—C₁-C₄ alkyl)ₙ-O—C₁-C₄ alkyl or
   d) branched or straight-chain C₂-C₅ alkynyl,
X₁ and X₂ independently of one another represent
   a) branched or straight-chain C₁-C₅ alkyl,
   b) substituted or unsubstituted aryl,
   c) substituted or unsubstituted arylalkyl or
   d) substituted or unsubstituted heteroaryl,
Z₂ represents a metal cation equivalent,
where n=0, 1, 2 or 3 and
all diastereomers and enantiomers are included.
46) Compounds according to claim 45, characterized in that A₂ represents hydroxyl, branched or straight-chain C₁-C₅ alkoxy or NH₂.
47) Compounds according to claim 46, characterized in that A₂ represents NH₂.
48) Compounds according to any of claims 45 to 47, characterized in that R⁸ and R⁹ are selected from the group consisting of hydrogen, substituted or unsubstituted ¹⁸F—C₆-C₁₀ aryl, substituted or unsubstituted ¹⁸F—C₅-C₁₀ heteroaryl, and substituted or unsubstituted ¹⁸F—C₃-C₆ cycloalkyl, with the proviso that one of the substituents R⁸ or R⁹ contains exactly one ¹⁸F isotope and the respective other substituent is hydrogen.
49) Compounds according to any of claims 45 to 48, characterized in that G₂ is selected from the group consisting of hydroxyl, branched or straight-chain C₁-C₄ alkoxy and OZ₂.
50) Compounds according to any of claims 45 to 49, characterized in that G₂ is methoxy.
51) Compounds according to any of claims 45 to 50, characterized in that Z₂ is selected from the group consisting of Na⁺, K⁺, Ca²⁺ and Mg²⁺.
52) Compounds according to any of claims 45 to 51, characterized in that Q₁ is selected from the group consisting of N(H)-tert-butoxycarbonyl, N(H)-benzyloxycarbonyl and

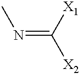

in which
X₁ and X₂ independently of one another represent
   a) branched or straight-chain C₁-C₅ alkyl,
   b) substituted or unsubstituted aryl,
   c) substituted or unsubstituted arylalkyl or
   d) substituted or unsubstituted heteroaryl
53) Compounds according to any of claims 45 to 52, characterized in that Q₁ represents N(H)-tert-butoxycarbonyl or N(H)-trityl.
54) Compounds according to claim 45:

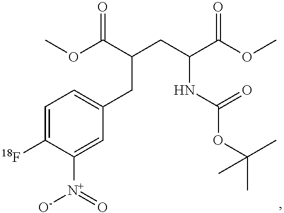

55) Compounds according to any of claims 45 to 54 for use as medicaments.
56) Compounds according to any of claims 45 to 54 for use for imaging in tumour disorders.
57) Use of compounds according to any of claims 45 to 54 for preparing a medicament for imaging in tumour disorders.
58) Process for preparing compounds of the general formula (VI) according to any of claims 45 to 54, which comprises
   reacting a compound of the formula (VII) according to claim 59 with F-18 fluoride.
59) Compounds of the formula (VII)

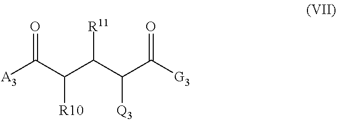

in which
A₃ represents
   a) branched or straight-chain C₁-C₅ alkoxy,
   b) branched or straight-chain hydroxy C₁-C₅ alkoxy,
   c) branched or straight-chain O—C₁-C₅ alkyl-(O—C₁-C₄ alkyl)ₙ-O—C₁-C₄ alkyl,
   d) N(C₁-C₅ alkyl)₂,
   e) NH₂,
   f) N(H)-L₃ or
   g) O-L₃,
G₃ represents
   a) O—Z₃,
   b) branched or straight-chain O—C₁-C₅ alkyl,
   c) branched or straight-chain O—C₂-C₅ alkenyl,
   d) branched or straight-chain O—C₁-C₅ alkyl-(O—C₁-C₄ alkyl)ₙ-O—C₁-C₄ alkyl,
   e) branched or straight-chain O—C₁-C₅ alkynyl or
   f) triphenylmethoxy,
R¹⁰ and R¹¹ represent
   a) hydrogen,
   b) substituted or unsubstituted E₁-C₆-C₁₀ mono- or bicyclic aryl,
   c) substituted or unsubstituted E₁-C₅-C₁₀ mono- or bicyclic heteroaryl,
   d) substituted or unsubstituted E₁-C₃-C₆ cycloalkyl,
   e) hydroxyl,
   f) branched or straight-chain C₁-C₅ alkyl or
   g) branched or straight-chain C₁-C₅ alkoxy,
   with the proviso that exactly one of the substituents R¹⁰ or R¹¹ contains an E₁ and the respective other substituent contains no E₁,
E₁ represents
   a) chloro,
   b) bromo,
   c) methanesulphonyloxy,
   d) trifluoromethanesulphonyloxy,
   e) nonafluorobutyloxy,
   f) tosyloxy or
   g) iodo,
Q₂ represents
   a) N(H)-tert-butoxycarbonyl
   b) N(H)-allyloxycarbonyl,
   c) N(H)-benzyloxycarbonyl,
   d) N(H)-ethoxycarbonyl,
   e) N(H)-methoxycarbonyl,
   f) N(H)-propoxycarbonyl,
   g) N(H)-2,2,2-trichloroethoxycarbonyl,
   h) N(H)-1,1-dimethylpropynyl,
   i) N(H)-1-methyl-1-phenylethoxycarbonyl,
   j) N(H)-1-methyl-1-(4-biphenylyl)ethoxycarbonyl,
   k) N(H)-cyclobutylcarbonyl,
   l) N(H)-1-methylcyclobutylcarbonyl,
   m) N(H)-vinylcarbonyl,
   n) N(H)-allylcarbonyl,
   o) N(H)-adamantylcarbonyl,
   p) N(H)-diphenylmethylcarbonyl,
   q) N(H)-cinnamylcarbonyl,
   r) N(H)-formyl,
   s) N(H)-benzoyl,
   t) N(H)-trityl,
   u) N(H)-p-methoxyphenyldiphenylmethyl,
   v) N(H)-di(p-methoxyphenyl)phenylmethyl,
   w)

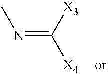

x) N-(tert-butoxycarbonyl)₂,
L₃ represents
   a) branched or straight-chain C₁-C₅ alkyl,
   b) branched or straight-chain C₂-C₅ alkenyl,
   c) branched or straight-chain C₁-C₅ alkyl-(O—C₁-C₄ alkyl)ₙ-O—C₁-C₄ alkyl or
   d) branched or straight-chain C₂-C₅ alkynyl,
X₃ and X₄ independently of one another represent
   a) branched or straight-chain C₁-C₅ alkyl,
   b) substituted or unsubstituted aryl,
   c) substituted or unsubstituted alkylaryl or
   d) substituted or unsubstituted heteroaryl,
Z₃ represents a metal cation equivalent,
where n=0, 1, 2 or 3 and
all diastereomers and enantiomers are included.
60) Use of compounds of the formula (VII) for preparing compounds of the formula (V) or (VI):

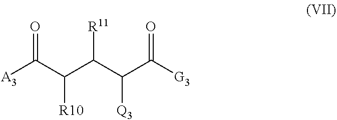

in which
A₃ represents
   a) branched or straight-chain C₁-C₅ alkoxy,
   b) branched or straight-chain hydroxy C₁-C₅ alkoxy,
   c) branched or straight-chain O—C₁-C₅ alkyl-(O—C₁-C₄ alkyl)ₙ-O—C₁-C₄ alkyl,
   d) N(C₁-C₅ alkyl)₂,
   e) NH₂,
   f) N(H)-L₃ or
   g) O-L₃,
G₃ represents
   a) O—Z₃,
   b) branched or straight-chain O—C₁-C₅ alkyl,
   c) branched or straight-chain O—C₂-C₅ alkenyl,
   d) branched or straight-chain O—C₁-C₅ alkyl-(O—C₁-C₄ alkyl)ₙ-O—C₁-C₄ alkyl,
   e) branched or straight-chain O—C₁-C₅ alkynyl or
   f) triphenylmethoxy,
R¹⁰ and R¹¹ represent
   a) hydrogen,
   b) substituted or unsubstituted E₁-C₆-C₁₀ mono- or bicyclic aryl,
   c) substituted or unsubstituted E₁-C₅-C₁₀ mono- or bicyclic heteroaryl,
   d) substituted or unsubstituted E₁-C₃-C₆ cycloalkyl,
   e) hydroxyl,
   f) branched or straight-chain C₁-C₅ alkyl or
   g) branched or straight-chain C₁-C₅ alkoxy,
   with the proviso that exactly one of the substituents Rⁿ or R^{H} contains an E₁ and the respective other substituent contains no E₁,
E₁ represents
   a) chloro,
   b) bromo,
   c) methanesulphonyloxy,
   d) trifluoromethanesulphonyloxy,
   e) nonafluorobutyloxy,
   f) tosyloxy or
   g) iodo,
Q₂ represents
   a) N(H)-tert-butoxycarbonyl
   b) N(H)-allyloxycarbonyl,
   c) N(H)-benzyloxycarbonyl,
   d) N(H)-ethoxycarbonyl,
   e) N(H)-methoxycarbonyl,
   f) N(H)-propoxycarbonyl,
   g) N(H)-2,2,2-trichloroethoxycarbonyl,
   h) N(H)-1,1-dimethylpropynyl,
   i) N(H)-1-methyl-1-phenylethoxycarbonyl,
   j) N(H)-1-methyl-1-(4-biphenylyl)ethoxycarbonyl,
   k) N(H)-cyclobutylcarbonyl,
   l) N(H)-1-methylcyclobutylcarbonyl,
   m) N(H)-vinylcarbonyl,
   n) N(H)-allylcarbonyl,
   o) N(H)-adamantylcarbonyl,
   p) N(H)-diphenylmethylcarbonyl,
   q) N(H)-cinnamylcarbonyl,
   r) N(H)-formyl,
   s) N(H)-benzoyl,
   t) N(H)-trityl,
   u) N(H)-p-methoxyphenyldiphenylmethyl,
   v) N(H)-di(p-methoxyphenyl)phenylmethyl,
   w)

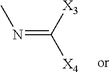

x) N-(tert-butoxycarbonyl)₂,
L₃ represents
   a) branched or straight-chain C₁-C₅ alkyl,
   b) branched or straight-chain C₂-C₅ alkenyl,
   c) branched or straight-chain C₁-C₅ alkyl-(O—C₁-C₄ alkyl)ₙ-O—C₁-C₄ alkyl or
   d) branched or straight-chain C₂-C₅ alkynyl,
X₃ and X₄ independently of one another represent
   a) branched or straight-chain C₁-C₅ alkyl,
   b) substituted or unsubstituted aryl,
   c) substituted or unsubstituted alkylaryl or
   d) substituted or unsubstituted heteroaryl,
Z₃ represents a metal cation equivalent,
where n=0, 1, 2 or 3 and
all diastereomers and enantiomers are included.
61) Compounds of the formula (VIII)

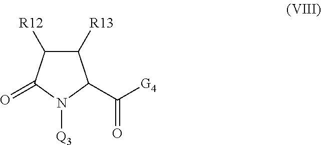

in which
G₄ represents
   a) branched or straight-chain O—C₁-C₅ alkyl,
   b) branched or straight-chain O—C₁-C₅ alkenyl,
   c) branched or straight-chain O—C₁-C₅ alkyl-(O—C₁-C₄ alkyl)ₙ-O—C₁-C₄ alkyl,
   d) branched or straight-chain O—C₂-C₅ alkynyl or
   e) triphenylmethoxy,
R¹² and R¹³ represent
   a) hydrogen,
   b) hydroxyl,
   c) branched or straight-chain C₁-C₅ alkyl,
   d) branched or straight-chain C₁-C₅ alkoxy or
   e) R¹⁴-E₂,
   with the proviso that exactly one of the substituents R¹² or R¹³ contains an E₂ and the respective other substituent contains no E₂,
E₂ represents
   a) chloro,
   b) bromo,
   c) methanesulphonyloxy,
   d) trifluoromethanesulphonyloxy,
   e) nonafluorobutyloxy,
   f) tosyloxy or
   g) iodo,
R¹⁴ represents
   a) substituted or unsubstituted C₆-C₁₀ mono- or bicyclic aryl,
   b) substituted or unsubstituted C₅-C₁₀ mono- or bicyclic heteroaryl,
   c) substituted or unsubstituted C₃-C₆ cyclo-alkyl,
Q₃ represents
   a) N-tert-butoxycarbonyl
   b) N-allyloxycarbonyl,
   c) N-benzyloxycarbonyl,
   d) N-ethoxycarbonyl,
   e) N-methoxycarbonyl,
   f) N-propoxycarbonyl,
   g) N-2,2,2-trichloroethoxycarbonyl,
   h) hydrogen,
   i) N-1-methyl-1-phenylethoxycarbonyl,
   j) N-1-methyl-1-(4-biphenylyl)ethoxycarbonyl,
   k) N-cyclobutylcarbonyl,
   l) N-1-methylcyclobutylcarbonyl,
   m) N-vinylcarbonyl,
   n) N-allylcarbonyl,
   o) N-adamantylcarbonyl,
   p) N-diphenylmethylcarbonyl,
   q) N-cinnamylcarbonyl,
   r) N-formyl or
   s) N-benzoyl,
   t) N(H)-trityl,
   u) N(H)-p-methoxyphenyldiphenylmethyl,
   v) N(H)-di(p-methoxyphenyl)phenylmethyl,
   w)

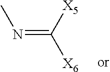

x) N-(tert-butoxycarbonyl)₂,
X₅ and X₆ independently of one another represent
   a) branched or straight-chain C₁-C₅ alkyl,
   b) substituted or unsubstituted aryl,
   c) substituted or unsubstituted alkylaryl or
   d) substituted or unsubstituted heteroaryl,
where n=0, 1, 2 or 3 and
all diastereomers and enantiomers are included.
62) Use of compounds of the formula (VIII) for preparing compounds of the formula (V) or (VI):

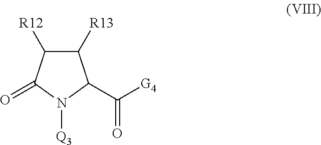

in which
G₄ represents
   a) branched or straight-chain O—C₁-C₅ alkyl,
   b) branched or straight-chain O—C₂-C₅ alkenyl,
   c) branched or straight-chain O—C₁-C₅ alkyl-(O—C₁-C₄ alkyl)ₙ-O—C₁-C₄ alkyl,
   d) branched or straight-chain O—C₁-C₅ alkynyl or
   e) triphenylmethoxy,
R¹² and R¹³ represent
   a) hydrogen,
   b) hydroxyl,
   c) branched or straight-chain C₁-C₅ alkyl,
   d) branched or straight-chain C₁-C₅ alkoxy or
   e) R¹⁴-E₂,
   with the proviso that exactly one of the substituents R¹² or R¹³ contains an E₂ and the respective other substituent contains no E₂,
E₂ represents
   a) chloro,
   b) bromo,
   c) methanesulphonyloxy,
   d) trifluoromethanesulphonyloxy,
   e) nonafluorobutyloxy,
   f) tosyloxy or
   g) iodo,
R₁₄ represents
   a) substituted or unsubstituted C₆-C₁₀ mono- or bicyclic aryl,
   b) substituted or unsubstituted C₅-C₁₀ mono- or bicyclic heteroaryl,
   c) substituted or unsubstituted C₃-C₆ cyclo-alkyl,
Q₃ represents
   a) N-tert-butoxycarbonyl
   b) N-allyloxycarbonyl,
   c) N-benzyloxycarbonyl,
   d) N-ethoxycarbonyl,
   e) N-methoxycarbonyl,
   f) N-propoxycarbonyl,
   g) N-2,2,2-trichloroethoxycarbonyl,
   h) hydrogen,
   i) N-1-methyl-1-phenylethoxycarbonyl,
   j) N-1-methyl-1-(4-biphenylyl)ethoxycarbonyl,
   k) N-cyclobutylcarbonyl,
   l) N-1-methylcyclobutylcarbonyl,
   m) N-vinylcarbonyl,
   n) N-allylcarbonyl,
   o) N-adamantylcarbonyl,
   p) N-diphenylmethylcarbonyl,
   q) N-cinnamylcarbonyl,
   r) N-formyl,
   s) N-benzoyl,
   t) N(H)-trityl,
   u) N(H)-p-methoxyphenyldiphenylmethyl,
   v) N(H)-di(p-methoxyphenyl)phenylmethyl,
   w)

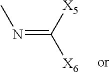

x) N-(tert-butoxycarbonyl)₂,
X₅ and X₆ independently of one another represent
   a) branched or straight-chain C₁-C₅ alkyl,
   b) substituted or unsubstituted aryl,
   c) substituted or unsubstituted alkylaryl or
   d) substituted or unsubstituted heteroaryl,
where n=0, 1, 2 or 3 and
all diastereomers and enantiomers are included.
63) Imaging-kit, comprising compounds of the general formula VII or VIII.
64) Pharmaceutical composition, comprising compounds of the general formula V, VI, VII or VIII and suitable pharmaceutical carrier substances.
65) Compounds according to any of claims 1 to 8, 13 to 22, 33 to 40 and 45 to 54, characterized in that the compounds are suitable for imaging in a dosage range of 37-600 MBq.
66) Compounds according to claim 65, characterized in that the compounds are particularly suitable in a dosage range of 150 MBq-370 MBq.

The term “aryl”, used herein on its own or as part of another group, refers to mono- or bicyclic aromatic groups which may contain 6 to 12 carbon atoms in the ring, such as, for example, phenyl or naphthyl, and in which they have any substitution.

The aryl groups may be substituted in any position leading to a stable compound, by one or more radicals from the group consisting of: hydroxyl, halogen, C₁-C₅-alkyl, C₁-C₅-alkoxy, cyano CF₃, and nitro.

Substituents which may be mentioned are methoxy, ethoxy, propoxy, isopropoxy, hydroxyl, fluorine, chlorine, bromine, methyl, ethyl, propyl, isopropyl or trifluoromethyl groups.

In each case, halogen is to be understood as meaning fluorine, chlorine, bromine or iodine.

The term “alkyl”, used herein on its own or as part of another group, refers to saturated carbon chains which may be straight-chain or branched, in particular to methyl, ethyl, n-propyl, isopropyl, n-butyl, isobutyl, tert-butyl or n-pentyl, 2,2-dimethylpropyl, 2-methyl-butyl or 3-methylbutyl, n-hexyl, n-heptyl, n-octyl, n-nonyl or n-decyl groups.

C₆-C₁₀-alkyl is optionally interrupted by one or more O, S or N.

Alkenyl substituents are in each case straight-chain or branched, including, for example, the following radicals: vinyl, propen-1-yl, propen-2-yl, but-1-en-1-yl, but-1-en-2-yl, but-2-en-1-yl, but-2-en-2-yl, 2-methylprop-2-en-1-yl, 2-methylprop-1-en-1-yl, but-1-en-3-yl, but-3-en-1-yl, allyl.

The alkynyl groups can be straight-chain or branched and are, for example, ethynyl, —CH₂—C≡CH, —CH₂—C≡CH, —C≡C—CH₃, —CH(CH₃)—C≡CH, —C≡C—CH₂(CH₃), —C(CH₃)₂C≡CH, —C≡C—CH(CH₃)₂—, —CH(CH₃)—C≡C—CH₃, —CH₂—C≡C—CH₂(CH₃).

Halogen represents fluoro, chloro, bromo and iodo. Preference is given to chloro, bromo and iodo.

The C₁-C₅-alkoxy groups can be straight-chain or branched and may represent a methoxy, ethoxy, n-propoxy, isopropoxy, n-butoxy, isobutoxy, tert-butoxy or n-pentoxy, 2,2-dimethylpropoxy, 2-methylbutoxy or 3-methylbutoxy group.

The heteroaryl radical comprises in each case 5-10 ring atoms and may, instead of carbon atoms, contain one or more identical or different heteroatoms, such as oxygen, nitrogen or sulphur, in the ring, and may additionally in each case be benzo-fused.

Examples which may be mentioned are:
thienyl, furanyl, pyrrolyl, oxazolyl, thiazolyl, imidazolyl, pyrazolyl, isoxazolyl, isothiazolyl, oxadiazolyl, triazolyl, thiadiazolyl, etc.
and
benzo derivatives thereof, such as, for example,
benzofuranyl, benzothienyl, benzothiazole, benzoxazolyl, benzimidazolyl, indazolyl, indolyl, isoindolyl, etc.;
or
pyridyl, pyridazinyl, pyrimidinyl, pyrazinyl, triazinyl, etc.

Compounds according to the invention in which the [F-18]-isotope is positioned via an alkoxy group in the 4-position of the glutamic acid skeleton, such as, for example, in 4-[F-18]fluorohexoxyglutamic acid (1), can be prepared as shown in Scheme 9. Thus, for example, the acidic removal of the protective groups of the compound 2 or (3) affords the compound 4-[F-18]fluoro-hexoxyglutamic acid (1) according to the invention.

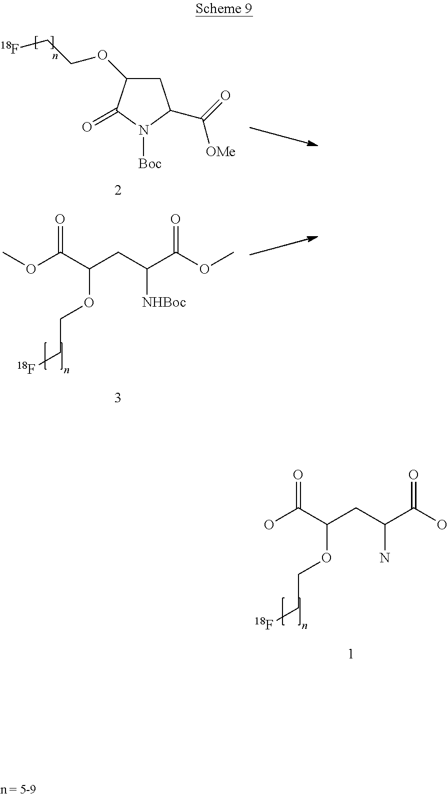

Here, various organic (for example trifluoroacetic acid), but especially inorganic acids, such as, for example, hydrobromic acid, hydrochloric acid, sulphuric acid, perchloric acid or phosphoric acid may be used. Also possible is a basic ring opening of 2 using lithium hydroxide, sodium hydroxide, potassium hydroxide, etc. (S. Baker et al. Tetrahedron Lett. 1998, 39, 2815-2818).

The compound 1 according to the invention of the formula (I) can be purified by HPLC, where, in principle, various purification steps may be carried out upstream or downstream, such as, for example, purification on a RP-C18 cartridge or other separating materials.

The radiochemical fluorination of tosylate 4, which is synthesized analogously to the method described in the literature (N. Sharma et al. Tetrahedron Lett. 2004, 45, 1403-1406) from 5, to the [F-18]-labelled glutamic acid derivative 2 can be carried out using methods known to the person skilled in the art (see Scheme 10).

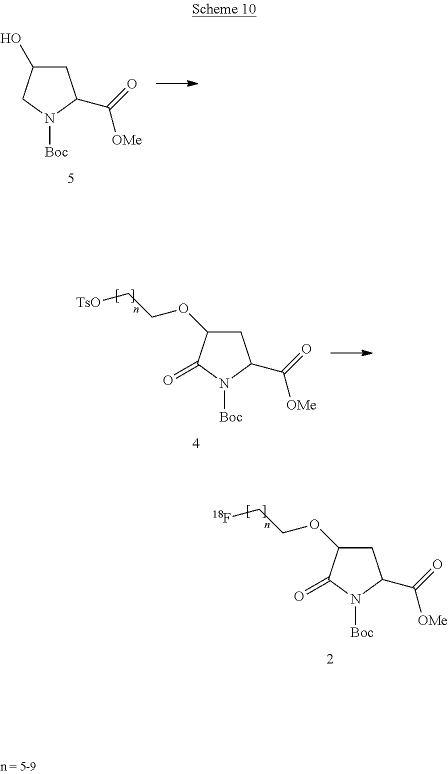

Here, compound 2 can be reacted in the presence of a base, such as, for example, tetraalkylammonium carbonate and tetraalkylphosphonium carbonate and potassium carbonate, etc., with the appropriate [F-18]-fluoride solution. The reaction is preferably carried out at elevated temperatures. The addition of crone ethers, such as, for example, Kryptofix (K2.2.2), may have a positive effect on the reaction, in particular in combination with K₂CO₃ as catalyzing base. Possible solvents are preferably aprotic, but it is also possible to use protic solvents or else aprotic solvent additives, such as, for example, water. Usually, acetonitrile, dimethyl sulphoxide or dimethylformamide are used as the most suitable solvents for the radio-chemical fluorination with [F-18]-fluoride anions. Usually, compound 2 does not have to be subjected to a purification but can be treated instantly using the methods described for the conversion of 2 into 1. However, a purification of the compound 2 is possible in principle, preferably using preparative HPLC with a nonpolar phase, such as, for example, RP C-18. Also possible is a purification using cartridges.

The radiochemical fluorination of tosylate 6, which is synthesized analogously to the method described in the literature (X. Zhang Tetrahedron Lett. 2001, 42, 5335-5338) from 4, to the [F 18]-labelled glutamic acid derivative 3 can be carried out by methods known to the person skilled in the art (see Scheme 11).

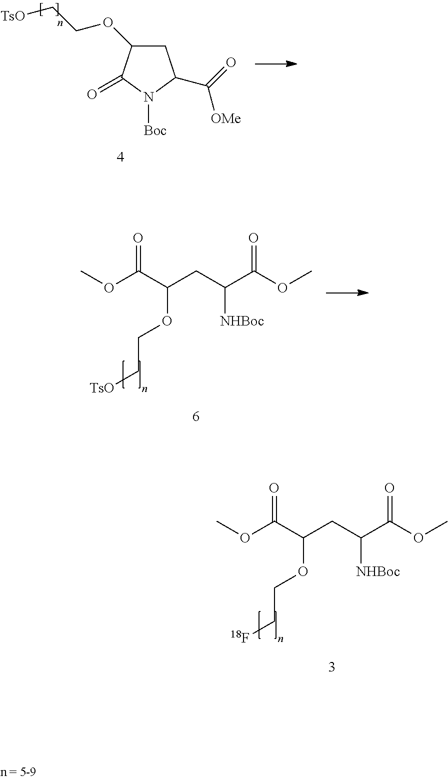

Here, compound 6 can be reacted in the presence of a base, such as, for example, tetraalkylammonium carbonate and tetraalkylphosphonium carbonate and potassium carbonate, etc., with the appropriate [F-18]-fluoride solution. The reaction is preferably carried out at elevated temperatures. The addition of crone ethers, such as, for example, Kryptofix (K2.2.2), may have a positive effect on the reaction, in particular in combination with K₂CO₃ as catalyzing base. Possible solvents are preferably aprotic, but it is also possible to use protic solvents or else aprotic solvent additives, such as, for example, water. Usually, acetonitrile, dimethyl sulphoxide or dimethylformamide are used as the most suitable solvents for the radio-chemical fluorination with [F-18]-fluoride anions. Usually, compound 3 does not have to be subjected to a purification but can be treated instantly using the methods described for the conversion of 3 into 1. However, a purification of the compound 3 is possible in principle, preferably using preparative HPLC with a nonpolar phase, such as, for example, RP C-18. Also possible is a purification using cartridges.

The synthesis of F-19 reference compounds 7, 8 and 9 can be carried out as shown in Scheme 12.

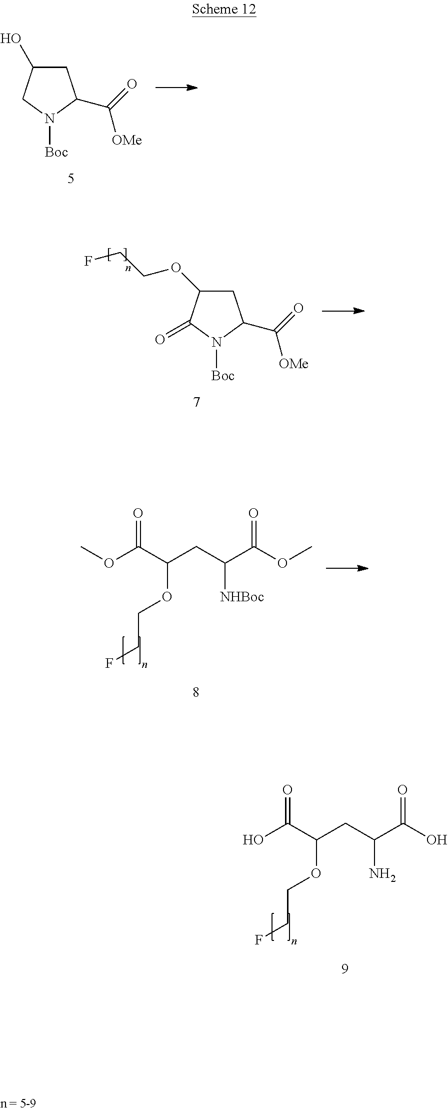

7 can be obtained by alkylating and oxidizing the hydroxyproline derivative 5. For preparing F-19 reference compounds, it has also been found to be advantageous to prepare the fluorides from analogous hydroxyl compounds using DAST (diethylaminosulphur trifluoride) according to methods known to the person skilled in the art, as described, for example, in Example 2c.

Ring-opening of the pyroglutamine derivative 7 gives the open-chain reference compound 8. The acidic removal of the protective groups leads to the glutamic acid derivative 9.

Compounds according to the invention in which the [F-18]-isotope is positioned via an alkyl group into the 4-position of the glutamic acid skeleton, such as, for example, 4-[F-18]fluorohexylglutamic acid (10) or 4-[F-18]fluorooctylglutamic acid (11), can be prepared as shown in Scheme 13. Thus, for example, the acidic removal of the protective groups of compounds 12 and 13 gives the compounds according to the invention 4-[F-18]fluorohexylglutamic acid (10) and 4-[F-18]-fluorooctylglutamic acid (11), respectively.

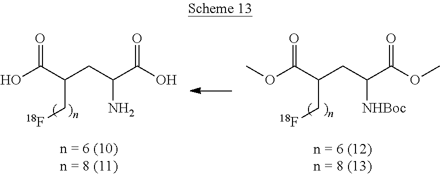

Here, various organic (for example trifluoroacetic acid), but especially inorganic acids, such as, for example, hydrobromic acid, hydrochloric acid, sulphuric acid, perchloric acid or phosphoric acid may be used. The compounds 10 and 11 according to the invention of the formula (I) can be purified by HPLC, where, in principle, various purification steps may be carried out upstream or downstream, such as, for example, purification using an RP-C18 cartridge or other separating materials.

The radiochemical fluorination of bromide 14 or tosylate 15, which are synthesized analogously to the method described in the literature (S. Hanessian, et al. J. Org. Chem. 2005, 70, 5070-5085) from 16, to the [F-18]-labelled glutamic acid derivatives 12 and 13 can be carried out by methods known to the person skilled in the art (see Scheme 14).

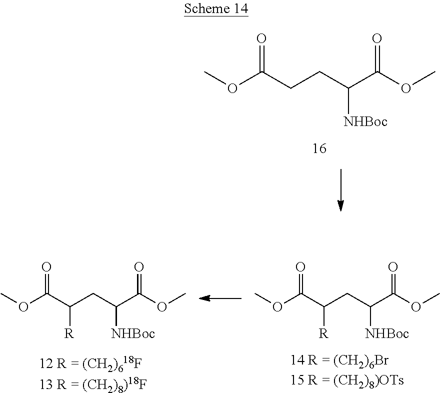

Here, compounds 14 and 15 can be reacted in the presence of a base, such as, for example, tetraalkylammonium carbonate and tetraalkylphosphonium carbonate and potassium carbonate, etc., with the appropriate [F-18]-fluoride solution. The reaction is preferably carried out at elevated temperatures. The addition of crone ethers, such as, for example, Kryptofix (K2.2.2), may have a positive effect on the reaction, in particular in combination with K₂CO₃ as catalyzing base. Possible solvents are preferably aprotic, but it is also possible to use protic solvents or else aprotic solvent additives, such as, for example, water. Usually, acetonitrile, dimethyl sulphoxide or dimethylformamide are used as the most suitable solvents for the radiochemical fluorination with [F-18]-fluoride anions. Usually, compounds 12 and 13 do not have to be subjected to a purification but can be treated instantly using the methods described for the conversion of 12 into 10 or 13 into 11. However, a purification of the compounds 12 and 13 is possible in principle, preferably using preparative HPLC with a nonpolar phase, such as, for example, RP C-18.

The F-19 reference compounds 17 and 18 can be synthesized by alkylation of the glutamic acid derivative 16 (Scheme 15).

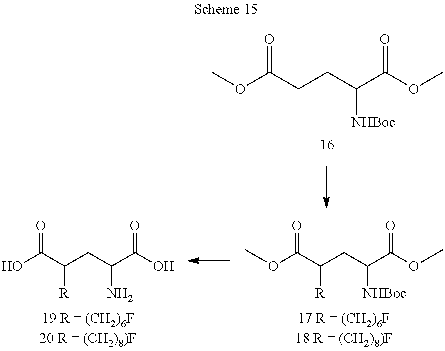

Compound 16 can also be alkylated using iodides, preferably diiodides, analogously to Example 1a. In this case, a precursor suitable for radiochemical fluorination is obtained in one step from the commercially available glutamic acid derivative 16.

Removal of the protective groups affords the fluoro-alkylated glutamic acid derivatives 19 and 20.

Suitable precursors according to the invention also include aromatic nitro compounds, as illustrated in Example 3.

The introduction of cycloalkyl substituents is carried out by processes known to the person skilled in the art, for example by alkylating the glutamic acid derivative 16 as described in Example 2a.

### EXAMPLES

### Example 1

### 2-Amino-4-(6-fluorohexyl)pentanedioic acid

### a) Dimethyl (2S,4S)-2-tert-butoxycarbonylamino-4-(6-iodohexyl)pentanedioate (precursor for (2S,4S)-2-amino-4-(6-fluorohexyl)pentanedioic acid)

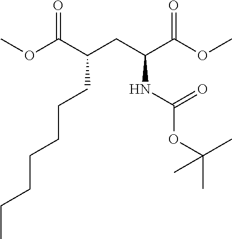

5.51 g (20 mmol) of dimethyl Boc-L-glutamate (Advanced Chemtech) are dissolved in 60 ml of tetrahydrofuran (THF) and cooled to −70° C. At this temperature, 44 ml (44 mmol) of a 1M solution of lithium bis(trimethyl-silyl)amide in THF are added dropwise over a period of one hour, and the mixture is stirred at −70° C. for another 2 hours. 20.28 g (60 mmol) of 1,6-diiodohexane are then added dropwise, and after 2 h at this temperature, the cooling bath is removed and 100 ml of 2N hydrochloric acid and 300 ml of ethyl acetate are added. The organic phase is separated off, washed with water until neutral, dried over sodium sulphate and filtered, and the filtrate is concentrated. The crude product obtained in this manner is chromatographed in silica gel using a hexane/ethyl acetate gradient, and the appropriate fractions are combined and concentrated.

Yield: 0.2 g (2.1%)

Elemental analysis:

| | | | | |
|---|---|---|---|---|
| calc. | C 44.54 | H 6.65 | I 26.15 | N 2.89 |
| found | C 44.28 | H 6.75 | I 25.83 | N 3.04 |

### b) Dimethyl (2S,4S)-2-tert-butoxycarbonylamino-4-(6-fluorohexyl)pentanedioate

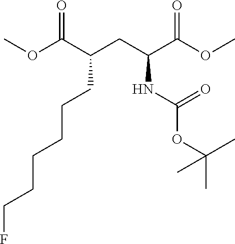

A solution of 152 mg (1.12 mmol) of silver fluoride in 1.5 ml of water is added to 0.49 g (1 mmol) of the compound described in Example 1a in 30 ml of acetonitrile, and the mixture is stirred at 40° C. overnight. The resulting suspension is filtered, the solution is evaporated to dryness and the crude product obtained in this manner is chromatographed on silica gel using a hexane/ethyl acetate gradient, and the appropriate fractions are combined and concentrated.

Yield: 132 mg (35%)

Elemental analysis:

| | | | | |
|---|---|---|---|---|
| calc. | C 57.28 | H 8.55 | F 5.03 | N 3.71 |
| found | C 57.03 | H 8.41 | F 4.80 | N 3.82 |

### c) (2S,4S)-2-Amino-4-(6-fluorohexyl)pentanedioic acid

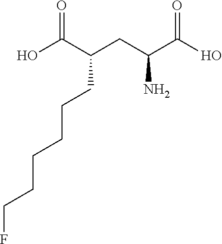

26.4 mg (0.07 mmol) of the compound described in Example 1b are dissolved in 2 ml of THF, 1 ml of 1N aqueous sodium hydroxide solution is added and the mixture is stirred at room temperature for 4 h. The mixture is then concentrated to dryness, and the resulting crude product is dissolved in about 20 ml of 3N hydrogen chloride in diethyl ether, stirred overnight, concentrated and repeatedly coevaporated with diethyl ether. The crude product obtained in this manner is chromatographed on C18 silica gel using a water/methanol gradient, and the appropriate fractions are combined and concentrated.

Yield: 5.8 mg (33%)

Elemental analysis:

| | | | | |
|---|---|---|---|---|
| calc. | C 53.00 | H 8.09 | F 7.62 | N 5.62 |
| found | C 52.68 | H 8.33 | F 7.24 | N 5.41 |

### d) Dimethyl (2S,4S)-2-tert-butoxycarbonylamino-4-(6-[F-18]fluorohexyl)pentanoate

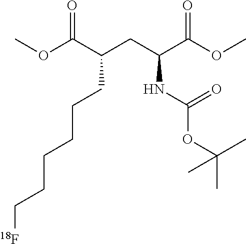

[F-18]-Fluoride was prepared by the [O-18](p,n)[F-18] reaction in a cyclotron. The isotope solution (5.3 GBq) was applied to a Sep-Pack Light QMA cartridge. The [F-18]-fluoride was eluted from the cartridge using a Kryptofix 2.2.2/K₂CO₃ solution (5 g K2.2.2, 1 mg K₂CO₃, MeCN (1.5 ml), water (0.5 ml). The solvent was removed at 120° C. in a stream of nitrogen with addition of acetonitrile (three times 1 ml).

5 mg (10.0 μmol) of dimethyl 2-tert-butoxycarbonyl-amino-4-[6-iodohexyl]pentanedioate (1a) in 1 ml of acetonitrile were added, and the resulting mixture was stirred at 110° C. for 10 min. After cooling to about 60° C., the mixture was passed through a Silica-Plus cartridge.

The intermediate was purified by HPLC(C18, acetonitrile/water. The HPLC fraction was diluted with water (about 50 ml) and passed through a C18 cartridge. The intermediate was eluted with 1 ml of acetonitrile. 1.3 GBq (41% d.c.) of dimethyl (2S,4S)-2-tert-butoxy-carbonylamino-4-(6-[F-18]fluorohexyl)pentanedioate (1d) were obtained in a synthesis time of 80 min.

### e) (2S,4S)-2-Amino-4-(6-[F-18]fluorohexyl)pentanedioic acid

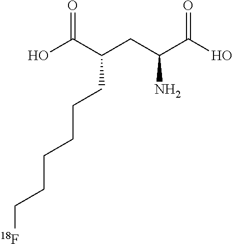

0.5 ml of 4N HCl was added to 1.2 GBq of dimethyl (2S,4S)-2-tert-butoxycarbonylamino-4-(6-[F-18]fluoro-hexyl)pentanoate (1d) in 1 ml of acetonitrile. With stirring, the mixture was heated at 130° C. (oil bath temperature) for 10 min. After cooling to room temperature, the solution was neutralized by addition of about 700 μl of 2N NaOH. This gave 1.0 GBq (83% d.c.) of (2S,4S)-2-amino-4-(6-[F-18]fluorohexyl)pentanedioic acid (1e).

### Example 2

### 2-Amino-4-(3-fluorocyclobutoxy)pentanedioic acid

### a) 1-tert-Butyl 2-methyl 4-(3-benzyloxycyclobutoxy)-5-oxopyrrolidine-1,2-dioate

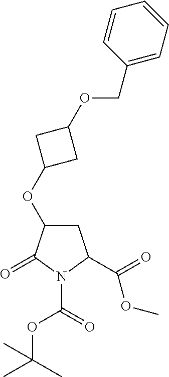

0.98 g (4 mmol) of 1-tert-butyl 2-methyl (2S,4S)-4-hydroxypyrrolidine-1,2-dioate (ALDRICH) is dissolved in 10 ml of dichloromethane and cooled in an ice-bath. After addition of 1.03 g of 3-benzyloxycyclobutyl methanesulphonate (WO 9937644 v. 21.1.1998) and 1.36 g (4 mmol) of tetrabutylammonium bisulphate, 18 ml of 50% strength aqueous sodium hydroxide solution are added and the mixture is stirred on ice for 2 hours and at room temperature overnight. After addition of 200 ml of water and 200 ml of dichloromethane, the organic phase is washed with water, dried over sodium sulphate and filtered, and the filtrate is concentrated. The crude product obtained in this manner is chromatographed in silica gel using a dichloromethane/ethyl acetate gradient, and the appropriate fractions are combined and concentrated.

Yield: 67 mg (4.0%)

Elemental analysis:

| | | | |
|---|---|---|---|
| calc. | C 62.99 | H 6.97 | N 3.98 |
| found | C 62.70 | H 7.21 | N 4.19 |

### b) 1-tert-Butyl 2-methyl 5-oxo-4-[3-(toluene-4-sulphonyloxy)cyclobutoxy]pyrrolidine-1,2-dioate

(precursor of 2-amino-4-(3-fluorocyclobutoxy)pentanedioic acid

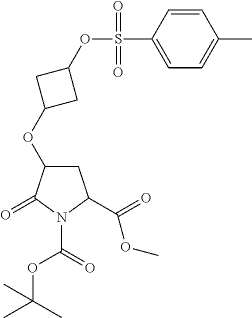

210 mg (0.5 mmol) of the compound described in Example 2a are dissolved in 5 ml of methanol and hydrogenated under a hydrogen atmosphere on a palladium on activated carbon (5%) catalyst. After 20 h, the catalyst is filtered off with suction and the solution is evaporated. The residue is dissolved in dichloro-methane and cooled in an ice-bath. After addition of 0.30 g (3 mmol) of triethylamine and 115 mg (1 mmol) of methanesulphonyl chloride, the mixture is stirred on ice for 2 h and concentrated, and the crude product obtained in this manner is chromatographed on silica gel using a hexane/ethyl acetate gradient, and the appropriate fractions are combined and concentrated.

Yield: 104 mg (43%)

Elemental analysis:

| | | | | |
|---|---|---|---|---|
| calc. | C 54.65 | H 6.05 | N 2.90 | S 6.63 |
| found | C 54.48 | H 6.19 | N 3.01 | S 6.18 |

### c) 2-Amino-4-(3-fluorocyclobutoxy)pentanedioic acid

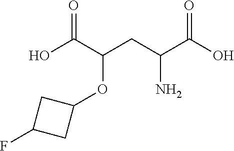

210 mg (0.5 mmol) of the compound described in Example 2a are dissolved in 5 ml of methanol and hydrogenated under a hydrogen atmosphere on a palladium on activated carbon (5%) catalyst. After 20 h, the catalyst is filtered off with suction and the solution is evaporated. The hydroxyl compound obtained is, without further characterization, dissolved in 10 ml of dichloromethane and cooled on ice. After addition of 0.16 g (1 mmol) of diethylaminosulphur trifluoride (DAST), the mixture is stirred on ice for 2 h and washed with water, and the organic phase is dried over sodium sulphate and filtered and the filtrate is concentrated. The crude product of the protected fluoride obtained in this manner is chromatographed on silica gel using a dichloromethane/ethyl acetate gradient, and the appropriate fractions are combined and concentrated.

The residue is dissolved in 2 ml of THF, 1 ml of 1N aqueous sodium hydroxide solution is added and the mixture is stirred at room temperature for 4 h. The mixture is then again concentrated to dryness, and the resulting diacid is dissolved in about 20 ml of 3N hydrogen chloride in diethyl ether, stirred overnight, concentrated and repeatedly coevaporated with diethyl ether. The crude product obtained in this manner is chromatographed on C18 silica gel using a water/methanol gradient, and the appropriate fractions are combined and concentrated.

Yield: 4.7 mg (4%)

Elemental analysis (calculated on the anhydrous compound):

| | | | | |
|---|---|---|---|---|
| calc. | C 45.96 | H 6.00 | F 8.08 | N 5.95 |
| found | C 45.62 | H 6.29 | F 7.87 | N 6.05 |

### d) 1-tert-Butyl 2-methyl 5-oxo-4-[3-[F-18]fluoro-cyclobut-1-oxy]pyrrolidine-1,2-dioate

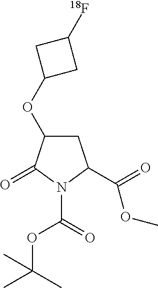

[F-18]-Fluoride was prepared by the [0-18](p,n)[F-18] reaction in a cyclotron. The isotope solution (3.27 GBq) was applied to a Sep-Pack Light QMA cartridge. The [F-18]-fluoride was eluted from the cartridge using a Kryptofix 2.2.2/K₂CO₃ solution (5 g K2.2.2, 1 mg K₂CO₃, MeCN (1.5 ml), water (0.5 ml). The solvent was removed at 120° C. in a stream of nitrogen with addition of acetonitrile (three times 1 ml). 5 mg (14.9 mmol) of 1-tert-butyl 2-methyl 4-[3-(toluene-4-sulphonyloxy)cyclobut-1-oxy]-5-oxopyrrolidine-1,2-dioate 2b in 1 ml of dimethylformamide were added, and the resulting mixture was stirred at 110° C. for 10 min. After cooling to about 60° C., the mixture was applied to a Silica-Plus cartridge.

The intermediate was purified by HPLC(C18, acetonitrile/water. The HPLC fraction was diluted with water (about 50 ml) and applied to a C18 cartridge. The intermediate was eluted with 1 ml of acetonitrile. 620 MBq (30% d.c.) of 1-tert-butyl 2-methyl 5-oxo-4-[3-[F-18]fluorocyclobut-1-oxy]pyrrolidine-1,2-dioate 2d were obtained in a synthesis time of 75 min.

### e) 2-Amino-4-(3-[F-18]fluorocyclobut-1-oxy)pentanedioic acid

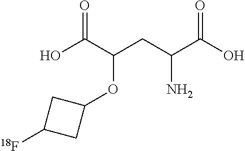

0.5 ml of 6N HCl was added to 620 MBq of 1-tert-butyl

2-methyl 5-oxo-4-[3-[F-18]fluorocyclobut-1-oxy]pyrrolidine-1,2-dioate 2d in 0.5 ml of acetonitrile. With stirring, the mixture was heated at 130° C. (oil bath temperature) for 10 min. After cooling to room temperature, the solution was neutralized by addition of about 600 μl of 4N NaOH. This gave 172 MBq (91% d.c.) of 2-amino-4-(3-[F-18]fluorocyclobut-1-oxy)pentanedioic acid 2e.

### Example 3

### 2-Amino-4-(4-fluoro-3-nitrobenzyl)pentanedioic acid

### a) Dimethyl 2-tert-butoxycarbonylamino-4-(3,4-di-nitrobenzyl)pentanedioate

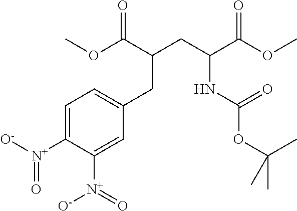

11.01 g (40 mmol) of dimethyl Boc-glutamate (Advanced Chemtech) are dissolved in 160 ml of tetrahydrofuran (THF) and cooled to −70° C. Over a period of one hour, 88 ml (88 mmol) of a 1M solution of lithium bis(tri-methylsilyl)amide in THF are added dropwise at this temperature, and the mixture is stirred at −70° C. for 2 hours. 20.88 g (80 mmol) of 4-bromomethyl-1,2-di-nitrobenzene in 250 ml of THF are then added dropwise, and after 2 h at this temperature the cooling bath is removed and 200 ml of 2N hydrochloric acid and 400 ml of ethyl acetate are added. The organic phase is separated off, washed with water until neutral, dried over sodium sulphate and filtered, and the filtrate is concentrated. The crude product obtained in this manner is chromatographed on silica gel using a hexane/ethyl acetate gradient, and the appropriate fractions are combined and concentrated.

Yield: 3.8 g (21%)

Elemental analysis:

| | | | |
|---|---|---|---|
| calc.: | C 50.11 | H 5.53 | N 9.23 |
| found: | C 49.94 | H 5.66 | N 9.40 |

### b) 2-Amino-4-(4-fluoro-3-nitrobenzyl)pentanedioic acid

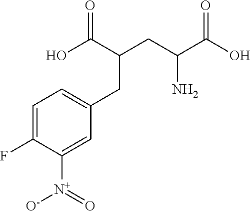

2.1 ml (2 mmol) of a 1M solution of tetrabutylammonium fluoride in THF (Aldrich) are added to a solution of 455 mg (1 mmol) of the compound described in Example 3a in 10 ml of dimethylformamide. After 2 h at room temperature, the mixture is evaporated to dryness, 5 ml and 5 ml of ethyl acetate are added, the organic phase is separated off, washed with water until neutral, dried over sodium sulphate and filtered, and the filtrate is concentrated. The crude product of the protected 4-fluoro-3-nitro compound obtained in this manner is chromatographed on silica gel using a hexane/ethyl acetate gradient, and the appropriate fractions are combined and concentrated.

The residue is dissolved in 5 ml of THF, 2 ml of 1N aqueous sodium hydroxide solution are added and the mixture is stirred at room temperature for 4 h. The mixture is then again concentrated to dryness, and the resulting diacid is dissolved in about 30 ml of 3N hydrogen chloride in diethyl ether, stirred overnight, concentrated and repeatedly coevaporated with diethyl ether. The resulting crude product is chromatographed on C18 silica gel using a water/methanol gradient, and the appropriate fractions are combined and concentrated.

Yield: 15 mg (5%)

Elemental analysis (calculated on the anhydrous compound):

| | | | | |
|---|---|---|---|---|
| calc.: | C 48.01 | H 4.36 | F 6.33 | N 9.33 |
| found: | C 47.76 | H 4.44 | F 6.02 | N 9.27 |

### c) Dimethyl 2-tert-butoxycarbonylamino-4-(4-[F-18]fluoro-3-nitrobenzyl)pentanedioate

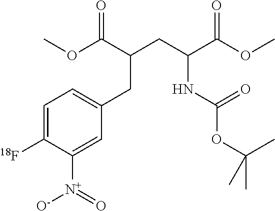

[F-18]-Fluoride was prepared by the [0-18](p,n)[F-18] reaction in a cyclotron. The isotope solution (5.72 GBq) was applied to a Sep-Pack Light QMA cartridge. The [F-18]-fluoride was eluted from the cartridge using a Kryptofix 2.2.2/K₂CO₃ solution (5 g K2.2.2, 1 mg K₂CO₃, MeCN (1.5 ml), water (0.5 ml). The solvent was removed at 120° C. in a stream of nitrogen with addition of acetonitrile (three times 1 ml). 5 mg (10.9 μmol) of dimethyl 2-tert-butoxycarbonyl-amino-4-(3,4-dinitrobenzyl)pentanedioate 3a in 1 ml of acetonitrile were added, and the resulting mixture was stirred at 130° C. for 15 min. After cooling to about 60° C., the mixture was passed through a silica-plus cartridge.

The intermediate was purified by HPLC(C18, acetonitrile/water). The HPLC fraction was diluted with water (about 50 ml) and passed through a C18 cartridge. The intermediate was eluted with 1 ml of acetonitrile. 1.2 GBq (35% d.c.) of dimethyl 2-tert-butoxycarbonyl-amino-4-(4-[F-18]fluoro-3-nitrobenzyl)pentanedioate 3c were obtained in a synthesis time of 80 min.

### d) 2-Amino-4-(4-[F-18]fluoro-3-nitrobenzyl)-pentanedioic acid

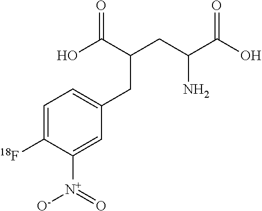

0.5 ml of 6N HCl was added to 1.1 GBq of dimethyl 2-tert-butoxycarbonylamino-4-(4-[F-18]fluoro-3-nitro-benzyl)pentanedioate 3c in 0.5 ml of acetonitrile. With stirring, the mixture was heated at 130° C. (oil bath temperature) for 10 min. After cooling to room temperature, the solution was neutralized by addition of about 600 μl of 4N NaOH. This gave 945 MBq (86% d.c.) of 2-amino-4-(4-[F-18]fluoro-3-nitrobenzyl)-pentanedioic acid 3d.

### Example 4

### a) 1-tert-Butyl 2-methyl 4-(6-iodohexyloxy)-5-oxo-pyrrolidine-1,2-dioate (precursor of 2-amino-4-(6-fluorohexyloxy)pentanedioic acid)

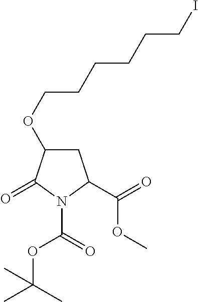

0.98 g (4 mmol) of 1-tert-butyl 2-methyl (2S,4S)-4-hydroxypyrrolidine-1,2-dioate (ALDRICH) are dissolved in 10 ml of dichloromethane and cooled in an ice-bath. After addition of 2.03 g (6 mmol) of 1,6-diiodohexane and 1.36 g (4 mmol) of tetrabutylammonium bisulphite, 18 ml of 50% strength aqueous sodium hydroxide solution are added, and the mixture is stirred on ice for 2 hours and at room temperature overnight. After addition of 200 ml of water and 200 ml of dichloro-methane, the organic phase is washed once more with water, dried over sodium sulphate and filtered, and the filtrate is concentrated. The residue is chromatographed on silica gel using a dichloromethane/ethyl acetate gradient, and the fractions comprising the desired alkylation product are combined and concentrated. The oil that remains is dissolved in 10 ml of ethyl acetate. After addition of 14 mg (0.06 mmol) of ruthenium(III) chloride hydrate, a solution of 0.32 g (1.5 mmol) of sodium periodate in 4 ml of water is added, the mixture is stirred overnight and diluted with ethyl acetate, the organic phase is washed with water, dried over sodium sulphate and filtered and the filtrate is concentrated. The crude product obtained in this manner is chromatographed on silica gel using a hexane/ethyl acetate gradient, and the appropriate fractions are combined and concentrated.

Yield: 394 mg (21%)

Elemental analysis:

| | | | | |
|---|---|---|---|---|
| calc.: | C 43.51 | H 6.01 | I 27.04 | N 2.98 |
| found: | C 43.22 | H 6.34 | I 26.59 | N 3.04 |

### b) 1-tert-Butyl 2-methyl 4-(6-fluorohexyloxy)-5-oxopyrrolidine-1,2-dioate

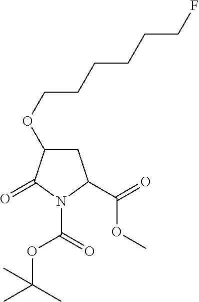

A solution of 152 mg (1.12 mmol) of silver fluoride and 1.5 ml of water is added to 0.47 g (1 mmol) of the compound described in Example 4a in 30 ml acetonitrile, and the mixture is stirred at 40° C. overnight. The resulting suspension is filtered, the solution is evaporated to dryness and the crude product obtained in this manner is chromatographed on silica gel using a hexane/ethyl ester gradient, and the appropriate fractions are combined and concentrated.

Yield: 90 mg (25%)

Elemental analysis:

| | | | | |
|---|---|---|---|---|
| calc.: | C 56.50 | H 7.81 | F 5.26 | N 3.88 |
| found: | C 56.25 | H 8.02 | F 5.11 | N 3.67 |

### c) 2-Amino-4-(6-fluorohexyloxy)pentanedioic acid

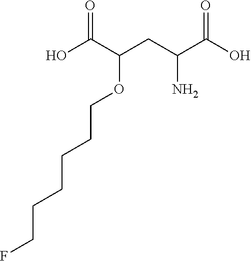

25.3 mg (0.07 mmol) of the compound described in Example 4c are dissolved in 2 ml of THF, 1 ml of 1N aqueous sodium hydroxide solution is added and the mixture is stirred at room temperature for 4 h. The mixture is then evaporated to dryness, and the resulting crude product is suspended in about 20 ml of 6N aqueous hydrochloric acid, stirred at 80° C. for 6 h and concentrated. The crude product obtained in this manner is chromatographed on C18 silica gel using a water/methanol gradient, and the appropriate fractions are combined and concentrated.

Yield: 3.5 mg (19%)

Elemental analysis (calculated on the anhydrous compound):

| | | | | |
|---|---|---|---|---|
| calc.: | C 49.80 | H 7.60 | F 7.16 | N 5.28 |
| found: | C 49.57 | H 7.80 | F 6.96 | N 5.33 |

### d) 1-tert-Butyl 2-methyl 4-(6-[F-18]fluorohexyl-oxy)-5-oxopyrrolidine-1,2-dioate

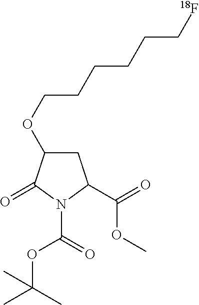

[F-18]-Fluoride was prepared by the [0-18](p,n)[F-18] reaction in a cyclotron. The isotope solution (3.8 GBq) was applied to a Sep-Pack Light QMA cartridge. The [F-18]-fluoride was eluted from the cartridge using a Kryptofix 2.2.2/K₂CO₃ solution (5 g K2.2.2, 1 mg K₂CO₃, MeCN (1.5 ml), water (0.5 ml). The solvent was removed at 120° C. in a stream of nitrogen with addition of acetonitrile (three times 1 ml).

5 mg (10.7 μmol) of 1-tert-butyl 2-methyl 4-(6-iodo-hexyloxy)-5-oxopyrrolidine-1,2-dioate 4a in 1 ml of acetonitrile were added, and the resulting mixture was stirred at 100° C. for 10 min. After cooling to about 60° C., the mixture was passed through a silica-plus cartridge.

The intermediate was purified by HPLC(C18, acetonitrile/water). The HPLC fraction was diluted with water (about 50 ml) and passed through a C18 cartridge. The intermediate was eluted with 1 ml of acetonitrile. 1.0 GBq (44% d.c.) of 1-tert-butyl 2-methyl 4-(6-[F-18]fluorohexyloxy)-5-oxopyrrolidine-1,2-dioate 4d were obtained in a synthesis time of 80 min.

### e) 2-Amino-4-(6-[F-18]fluorohexyloxy)pentanedioic acid

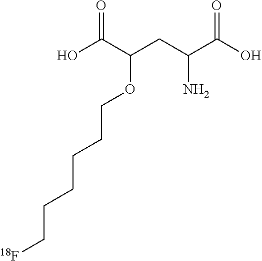

0.5 ml of 4N HCl was added to 1.0 GBq of 1-tert-butyl 2-methyl 4-(6-[F-18]fluorohexyloxy)-5-oxopyrrolidine-1,2-dioate 4d in 1 ml of acetonitrile. With stirring, the mixture was heated at 130° C. (oil bath temperature) for 10 min. After cooling to room temperature, the solution was neutralized by addition of about 700 μl of 2N NaOH.

This gave 900 MBq (90% d.c.) of 2-amino-4-(6-[F-18]fluorohexyloxy)pentanedioic acid 4e.

### Example 5

### Biological Characterization

The uptake of the glutamic acid derivatives according to the invention into tumour cells was investigated in cell experiments. Here, the uptake of a radiolabelled glutamic acid derivative (4R/S—[F-18]F-L-glutamic acid) was examined in the presence of the compounds according to the invention (competition experiments). The compounds according to the invention were employed in excess (1 mM) over 4R/S—[F-18]F-L-glutamic acid (tracer).

Native L-configured glutamic acid (L-Glu), which, at a concentration of 1 mM L-Glu causes an 87% inhibition of tracer uptake in the assay, was used as positive control.

Surprisingly, it was found that 4-(6-fluorohexyl)-L-Glu, too, leads to a 94% inhibition of 4R/S—[F-18]F-L-glutamic acid (tracer) uptake. Competition with (4S)-4-(3-fluorocyclobutoxy)-L-Glu likewise leads to >90% inhibition of tracer uptake.

TABLE 1
Inhibition of the uptake of 4R/S-[F-18]F-L-glutamic
acid by competition with compounds according to the
invention in the competition cell experiment.

| | % Tracer | |
|---|---|---|
| | uptake | S.D. |
| Control | 100.0 | 3.5 |
| L-Glu | 12.6 | 1.6 |
| 4-(R/S)-fluoro-D/L-Glu | 16.3 | 2.5 |
| (4S)-hydroxy-Glu | 13.2 | 1.8 |
| (4S)-methyl-Glu | 7.7 | 1.9 |
| (4S)-4-(3-fluoropropyl)-Glu | 5.4 | 2.2 |
| (4S)-4-(6-fluorohexyl)-Glu | 6.3 | 2.4 |
| (4S)-4-(3-fluorocyclobutoxy)-Glu | 5.7 | 1.3 |

(A549 cells, 10 min of incubation with 0.250 MBq of 4R/S-[F-18]F-L-Glu in PBS buffer, competitor concentration in each case 1 mM)

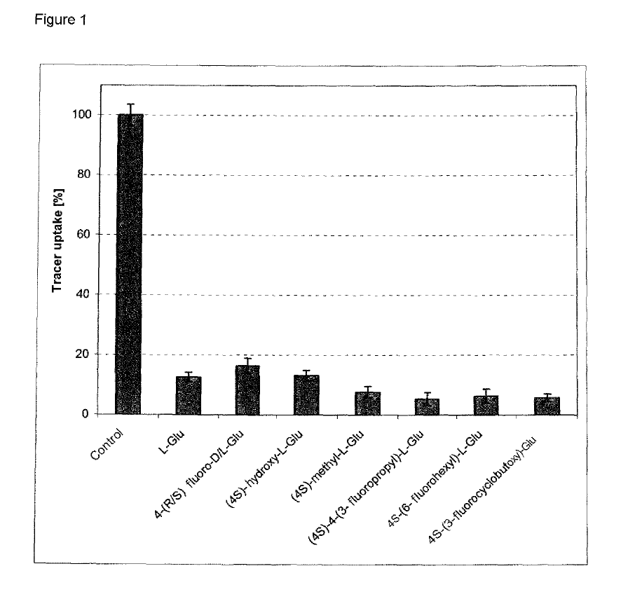

FIG. 1: graphic representation of the inhibition of the cellular uptake of 4R/S—[F-18]F-L-Glu by compounds according to the invention branched in the 4-position in A549 cells (human non-small-cell bronchial carcinoma) after 10 min of incubation.

Following labelling with F-18, (4S)-4-(6-[F-18]fluorohexyl)-L-Glu was examined in cell experiments with A460 tumour cells (human non-small-cell bronchial carcinoma). Here, it was possible to observe a time-dependent cellular uptake.

After 30 min of incubation, an uptake of 11.2% of the dose per 100 000 cells was measured for 4-(6-[F-18]fluorohexyl)-L-Glu. Accordingly, the accumulation was higher than that of [F-18]FDG (8.2%).

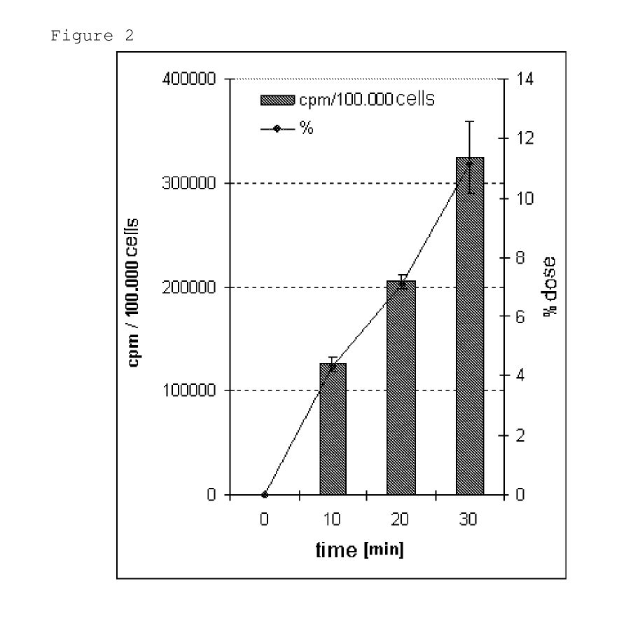

FIG. 2: time-dependent uptake of (4S)-4-(6-[F-18]fluorohexyl)-L-Glu in A549 cells. For all F-18-labelled compounds, a time-dependent intracellular radioactivity was observed. After 30 min, 11.2% of the dose of (4S)-4-(6-[F-18]fluorohexyl)-L-Glu had been taken up in 100 000 cells. As a comparison, 8.2% of the dose of [F-18]FDG are taken up under analogous conditions.

### Animal Experiments

(2S,4S)-2-Amino-4-(6-[F-18]fluorohexyl)pentanedioic acid was studied in rats bearing H460 tumours using PET imaging and subsequent organ distribution.

The highest enrichment at 0.52% of the injected dose per g in the tumour was measured at a time of 100 min after injection. In the PET study, a transient uptake or excretion in the kidneys or pancreas was observed. Thus, in these organs, an uptake of 0.36% ID/g and 0.20% ID/g, respectively, was observed after 100 min. Uptake into the bones was 0.43% ID/g. Thus, the tumour shows the greatest enrichment and could be visualized clearly using PET imaging.

TABLE 2
Organ distribution 100 min after i.v. administration of 15 MBq
of (2S,4S)-2-amino-4-(6-[F-18]fluorohexyl)pentanedioic acid
to mice/rats bearing H460 tumours, and PET imaging of 60-90 minutes.

| Organ | % ID/g | S.D. |
|---|---|---|
| Kidney | 0.36 | 0.05 |
| Pancreas | 0.20 | 0.04 |
| Liver | 0.08 | 0.01 |
| Bones | 0.43 | 0.04 |
| Muscle | 0.02 | 0.01 |
| Tumour | 0.52 | 0.06 |
| Blood | 0.04 | 0.01 |

### PET Using (4S)-4-(6-[F-18]Fluorohexyl)-L-Glu in Rats Bearing H460 Tumours

60 min after i.v. administration of 15 MBq of (2S,4S)-2-amino-4-(6-[F-18]fluorohexyl)pentanedioic acid to rats bearing H460 tumours, data acquisition with a PET scanner (Inveon) was started for 30 minutes. Image analysis shows a high uptake of (2S,4S)-2-amino-4-(6-[F-18]fluorohexyl)pentanedioic acid into the H460 tumour, and low enrichment in the remainder of the body.

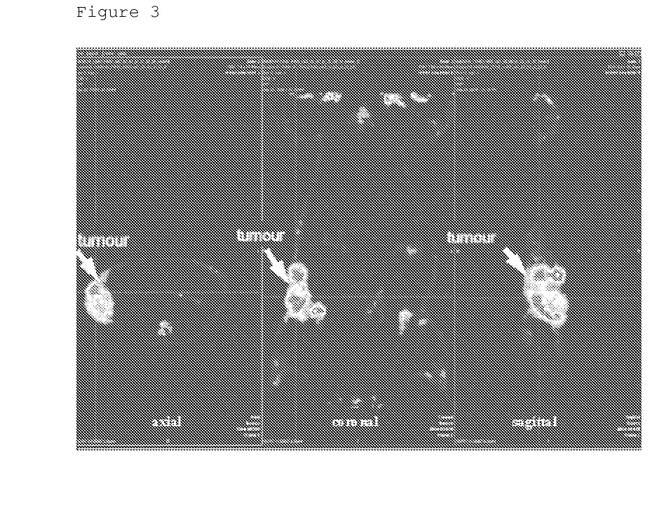

FIG. 3. PET study of 60 to 90 min after injection of (2S,4S)-2-amino-4-(6-[F-18]fluorohexyl)pentanedioic acid into mice bearing H460 tumours (analysis of sections)

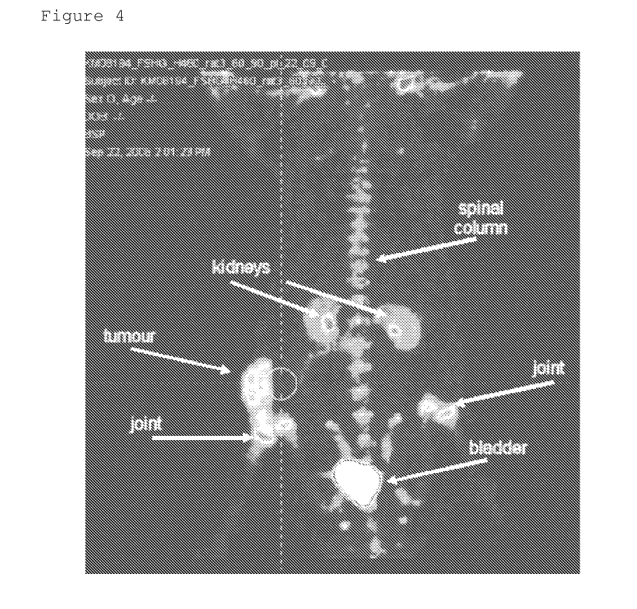

FIG. 4. PET study of 60 to 90 min after injection of (2S,4S)-2-amino-4-(6-[F-18]fluorohexyl)pentanedioic acid into mice bearing H460 tumours (whole body imaging)

### Example 6

### a) Di-tert-butyl (2S,4S)-2-tert-butoxycarbonyl-amino-4-(2-fluoro-5-trifluoromethylbenzyl)-pentanedioate

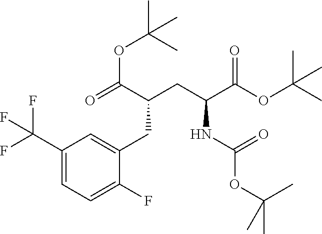

2.7 g (7.5 mmol) di-t-butyl Boc-glutamate (Journal of Peptide Research (2001), 58, 338) were dissolved in 30 ml of THF and cooled to −70° C. Over a period of 40 min, 16.5 ml (16.5 mmol) of 1M solution of lithium bis(trimethylsilyl)amide in THF were added dropwise at this temperature, and the mixture was stirred at −70° C. for another 2 h. 1.93 g (7.5 mmol) of 2-fluoro-5-trifluoromethylbenzyl bromide in 7 ml of THF were then added dropwise, and after 2 h at this temperature, 37.5 ml of 2N hydrochloric acid and 100 ml of dichloromethane are added. The organic phase was separated off, washed with water until neutral, dried over sodium sulphate and filtered, and the filtrate was concentrated. The crude product obtained in this manner was chromatographed on silica gel using a hexane/ethyl acetate gradient, and the appropriate fractions were combined and concentrated.

Yield: 2.3 g (57.3%)

MS (ESIpos): m/z=536 [M+H]⁺

1H NMR (300 MHz, CHLOROFORM-d) d ppm 1.03-1.50 (m, 27H) 1.80-2.00 (m, 2H) 2.60-3.10 (m, 3H) 4.05-4.30 (m, 1H) 4.85-4.95 (d, 1H) 7.05-7.15 (m, 1H) 7.40-7.55 (m, 2H)

### b) Neutral sodium salt of (2S,4S)-2-amino-4-(2-fluoro-5-trifluoromethylbenzyl)pentanedioic acid

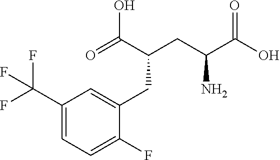

2.14 g (4 mmol) of the compound described in Example 6a were dissolved in 10 ml of THF, 50 ml of 2N hydrogen chloride in diethyl ether were added and the mixture was stirred at room temperature for 2 days. The mixture was then concentrated to dryness, the crude product obtained was re-distilled three times with diethyl ether and the residue was dissolved in about 10 ml of water and adjusted to pH 7.4 using 1N aqueous sodium hydroxide solution. The solution was freeze-dried and then chromatographed on C18 silica gel using a water/methanol gradient, and the appropriate fractions were combined and concentrated.

Yield: 1.25 g (9%)

MS (ESIpos): m/z=324 [M+H]⁺

1H NMR (300 MHz, D20) d ppm 1.97-2.20 (m, 2H) 3.02-3.08 (m, 3H) 3.72-3.78 (m, 1H) 7.25-7.32 (m, 1H) 7.62-7.68 (m, 2H)

### c) Di-tert-butyl (2S,4S)-2-tert-butoxycarbonyl-amino-4-(2-bromo-5-trifluoromethylbenzyl)pentanedioate

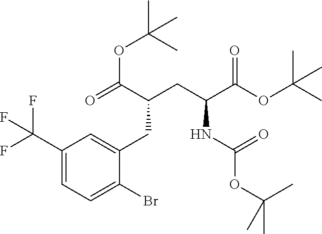

2.7 g (7.5 mmol) of di-t-butyl Boc-glutamate (Journal of Peptide Research (2001), 58, 338) were dissolved in 30 ml of THF and cooled to −70° C. Over a period of 40 min, 16.5 ml (16.5 mmol) of a 1M solution of lithium bis(trimethylsilyl)amide in THF were added dropwise at this temperature, and the mixture was stirred at −70° C. of another 2 h. A solution of 2.0 ml (6.3 mmol) of 2-bromo-5-trifluoromethylbenzyl bromide in 7 ml of THF was then added dropwise, and after 2 h at this temperature 37.5 ml of 2N hydrochloric acid and 100 ml of dichloromethane were added. The organic phase was separated off, washed with water until neutral, dried over sodium sulphate and filtered, and the filtrate was concentrated. The crude product obtained in this manner was chromatographed on silica gel using a hexane/ethyl acetate gradient, and the appropriate fractions were combined and concentrated.

Yield: 1.8 g (48%)

MS (ESIpos): m/z=596, 598 [M+H]⁺

1H NMR (300 MHz, CHLOROFORM-d) d ppm 1.03-1.50 (m, 27H) 1.80-2.20 (m, 2H) 2.60-3.10 (m, 3H) 4.05-4.25 (m, 1H) 4.85-4.95 (d, 1H) 7.30-7.40 (m, 1H) 7.45-7.50 (m, 1H) 7.60-7.70 (m, 1H)

### d) Di-t-butyl 2-tert-butoxycarbonylamino-4-(2-[F-18]fluoro-5-trifluoromethylbenzyl)-pentanedioate

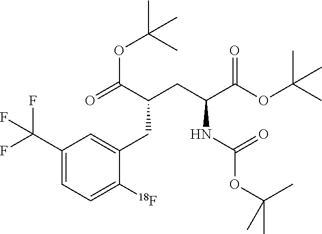

[F-18]fluoride was produced in a cyclotron via the [O-18](p,n)[F-18] reaction. The isotope solution (5.72 GBq) was applied to a Sep-Pack Light QMA cartridge. The [F-18]fluoride was eluted from the cartridge using a Kryptofix 2.2.2/K₂CO₃ solution (5 g K2.2.2, 1 mg K₂CO₃, MeCN (1.5 ml), water (0.5 ml)). The solvent was removed at 120° C. in a stream of nitrogen by adding acetonitrile (three times 1 ml).

5 mg (8.4 μmol) of dimethyl 2-tert-butoxycarbonylamino-4-(2-bromo-5-trifluoromethylbenzyl)pentanedioate 6c in 1 ml of acetonitrile/DMF (2:1) were added, and the resulting mixture was stirred at 130° C. for 15 min. After cooling to about 60° C., the mixture was passed through a Silica-Plus cartridge.

The intermediate was purified by HPLC(C18, acetonitrile/water). The HPLC fraction was diluted with water (about 50 ml) and passed through a C18 cartridge. The intermediate was eluted with 1 ml of acetonitrile. In a synthesis time of 90 min, 0.9 GBq (27.8% d.c.) of di-t-butyl 2-tert-butoxycarbonylamino-4-(2-[F-18]fluoro-5-trifluoromethylbenzyl)pentanedioate 6d were obtained.

### e) 2-Amino-4-(2-[F-18]fluoro-5-trifluoromethyl-benzyl)pentanedioic acid

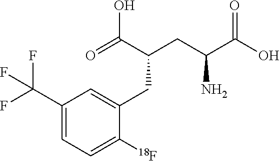

0.5 ml of 6N HCl was added to 0.8 GBq of di-t-butyl 2-tert-butoxycarbonylamino-4-(2-[F-18]fluoro-5-trifluoro-methylbenzyl)pentanedioate 6d in 0.5 ml of acetonitrile. With stirring, the mixture was heated at 130° C. (oil bath temperature) for 10 min. After cooling to room temperature, the solution was neutralized by addition of about 600 μl of 4N NaOH. This gave 745 MBq (93% d.c.) of 2-amino-4-(2-[F-18]fluoro-5-trifluoro-methylbenzyl)pentanedioic acid 6e.

1) Compounds of the general formula I 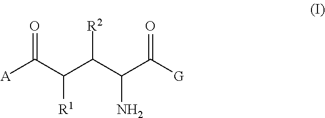
in which
A represents
a) hydroxyl,
b) branched or straight-chain C₁-C₅ alkoxy,
c) branched or straight-chain hydroxy C₁-C₅ alkoxy,
d) branched or straight-chain O—C₁-C₅ alkyl-(O—C₁-C₄ alkyl)ₙ-O—C₁-C₄ alkyl,
e) N(C₁-C₅ alkyl)₂,
f) NH₂,
g) N(H)-L,
h) O-L or
i) O—Z,
G represents
a) hydroxyl,
b) O—Z,
c) branched or straight-chain O—C₁-C₅ alkyl,
d) branched or straight-chain O—C₂-C₅ alkenyl,
e) branched or straight-chain O—C₁-C₅ alkyl-(O—C₁-C₄ alkyl)ₐ-O—C₁-C₄ alkyl,
f) branched or straight-chain O—C₂-C₅ alkynyl or
g) triphenylmethoxy,
R¹ and R² represent
a) hydrogen,
b) branched or straight-chain ¹⁸F—C₆-C₁₀ alkoxy,
c) branched or straight-chain ¹⁸F—C₆-C₁₀ alkyl,
d) branched or straight-chain ¹⁸F—C₆-C₁₀ alkenyl,
e) branched or straight-chain ¹⁸F—C₆-C₁₀ alkynyl,
f) hydroxyl,
g) branched or straight-chain C₁-C₅ alkyl or
h) branched or straight-chain C₁-C₅ alkoxy,
with the proviso that one of the substituents R¹ or R² contains exactly one ¹⁸F isotope and the respective other substituent contains no ¹⁸F isotope,
L represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₁-C₅ alkynyl, and
Z represents a metal cation equivalent,
where n=0, 1, 2 or 3 and
all diastereomers and enantiomers are included.

2) Compounds according to claim 1, selected from the group of compounds of the formulae:

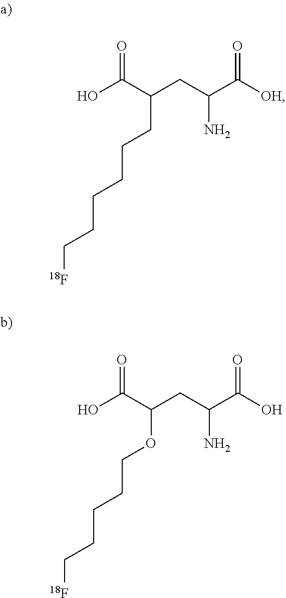

3) Process for preparing compounds of the general formula (I) according to claim 1,
which comprises
removing one or more protective groups present in a compound of the formula (II) 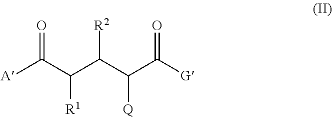
in which
A′ represents
a) hydroxyl,
b) branched or straight-chain C₁-C₅ alkoxy
c) branched or straight-chain hydroxy C₁-C₅ alkoxy,
d) branched or straight-chain O—C₁-C₅ alkyl-(O—C₁-C₄ alkyl-O—C₁-C₄ alkyl,
e) N(C₁-C₅ alkyl)₂,
f) NH₂,
g) N(H)-L′,
h) O-L′,
G′ represents
a) hydroxyl,
b) O—Z′,
c) branched or straight-chain O—C₁-C₅ alkyl,
d) branched or straight-chain O—C₂-C₅ alkenyl,
e) branched or straight-chain O—C₁-C₅ alkyl-(O—C₁-C₄ alkyl)ₙ-O—C₁-C₄ alkyl,
f) branched or straight-chain O—C₂-C₅ alkynyl or
g) triphenylmethoxy,
R¹ and R² represent
a) hydrogen,
b) branched or straight-chain ¹⁸F—C₆-C₁₀ alkoxy,
c) branched or straight-chain ¹⁸F—C₆-C₁₀ alkyl,
d) branched or straight-chain ¹⁸F—C₆-C₁₀ alkenyl,
e) branched or straight-chain ¹⁸F—C₆-C₁₀ alkynyl,
f) hydroxyl,
g) branched or straight-chain C₁-C₅ alkyl or
h) branched or straight-chain C₁-C₅ alkoxy,
with the proviso that exactly one of the substituents R¹ or R² contains exactly one ¹⁸F isotope and the respective other substituent contains no ¹⁸F isotope,
Q represents
a) N(H)-tert-butoxycarbonyl,
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
e) N(H)-2,2,2-trichloroethoxycarbonyl,
f) N(H)-1,1-dimethylpropynyl,
g) N(H)-1-methyl-1-phenylethoxycarbonyl,
h) N(H)-1-methyl-1-(4-biphenylyl)ethoxycarbonyl,
i) N(H)-cyclobutylcarbonyl,
j) N(H)-1-methylcyclobutylcarbonyl,
k) N(H)-vinylcarbonyl,
l) N(H)-allylcarbonyl,
m) N(H)-adamantylcarbonyl,
n) N(H)-diphenylmethylcarbonyl,
o) N(H)-cinnamylcarbonyl,
p) N(H)-formyl,
q) N(H)-benzoyl,
r) N(H)-trityl,
s) N(H)-n-methoxydiphenylmethyl,
t) N(H)-di(p-methoxyphenyl)phenylmethyl,
u) 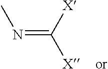
v) N-(tert-butoxycarbonyl)₂,
L′ represents
a) branched or straight-chain C₁-C₅ alkyl,
b) branched or straight chain C₁-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
X′ and X″ independently of one another represent
a) branched or straight-chain C₁-C₅ alkyl,
b) substituted or unsubstituted aryl,
c) substituted or unsubstituted aralkyl or
d) substituted or unsubstituted heteroaryl,
Z′ represents a metal cation equivalent,
where n=0, 1, 2 or 3 and
all diastereomers and enantiomers are included.

4) Compounds of the general formula (II): 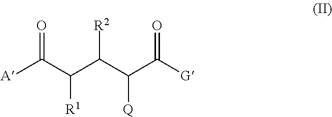
in which
A′ represents
a) hydroxyl,
b) branched or straight-chain C₁-C₅ alkoxy,
c) branched or straight-chain hydroxy C₁-C₅ alkoxy,
d) branched or straight-chain O—C₁-C₅ alkyl-(O—C₁-C₄ alkyl)ₙ-O—C₁-C₄ alkyl,
e) N(C₁-C₅ alkyl)₂,
f) NH₂,
g) N(H)-L′,
h) O-L′,
G′ represents
a) hydroxyl,
b) O—Z′,
c) branched or straight-chain O—C₁-C₅ alkyl,
d) branched or straight-chain O—C₂-C₅ alkenyl,
e) branched or straight-chain O—C₁-C₅ alkyl-(O—C₁-C₄ alkyl)ₙ-O—C₁-C₄ alkyl,
f) branched or straight-chain O—C₂-C₅ alkynyl or
g) triphenylmethoxy,
R¹ and R² represent
a) hydrogen,
b) branched or straight-chain ¹⁸F—C₆-C₁₀ alkoxy,
c) branched or straight-chain ¹⁸F—C₆-C₁₀ alkyl,
d) branched or straight-chain ¹⁸F—C₆-C₁₀ alkenyl,
e) branched or straight-chain ¹⁸F—C₆-C₁₀ alkynyl,
f) hydroxyl,
g) branched or straight-chain C₁-C₅ alkyl or
h) branched or straight-chain C₁-C₅ alkoxy,
with the proviso that exactly one of the substituents R¹ or R² contains exactly one ¹⁸F isotope and the respective other substituent contains no ¹⁸F isotope,
Q represents
a) N(H)-tert-butoxycarbonyl,
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
e) N(H)-2,2,2-trichloroethoxycarbonyl,
f) N(H)-1,1-dimethylpropynyl,
g) N(H)-1-methyl-1-phenylethoxycarbonyl,
h) N(H)-1-methyl-1-(4-biphenylyl)ethoxycarbonyl,
i) N(H)-cyclobutylcarbonyl,
j) N(H)-1-methylcyclobutylcarbonyl,
k) N(H)-vinylcarbonyl,
l) N(H)-allylcarbonyl,
m) N(H)-adamantylcarbonyl,
n) N(H)-diphenylmethylcarbonyl,
o) N(H)-cinnamylcarbonyl,
p) N(H)-formyl,
q) N(H)-benzoyl,
r) N(H)-trityl,
s) N(H)-p-methoxydiphenylmethyl,
t) N(H)-di(p-methoxyphenyl)phenylmethyl,
u) 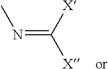
v) N-(tert-butoxycarbonyl)₂,
L′ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₁-C₅ alkynyl,
X′ and X″ independently of one another represent
a) branched or straight-chain C₁-C₅ alkyl,
b) substituted or unsubstituted aryl,
c) substituted or unsubstituted aralkyl or
d) substituted or unsubstituted heteroaryl,
Z′ represents a metal cation equivalent,
where n=0, 1, 2 or 3 and
all diastereomers and enantiomers are included.

5) Compounds according to claim 4, selected from the group of compounds of the formulae:

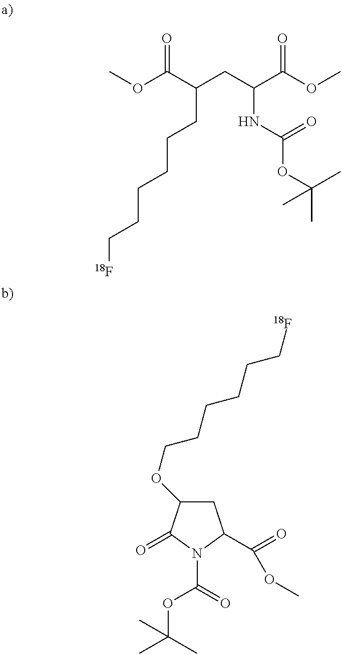

6) Process for preparing compounds of the general formula (II) according to claim 4,
which comprises
reacting a compound of the formula (III) with F-18 fluoride, 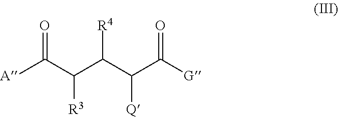
in which
A″ represents
a) branched or straight-chain C₁-C₅ alkoxy,
b) branched or straight-chain hydroxy C₁-C₅ alkoxy,
c) branched or straight-chain O—C₁-C₅ alkyl-(O—C₁-C₄ alkyl)ₙ-O—C₁-C₄ alkyl,
d) N(C₁-C₅ alkyl)₂,
e) NH₂,
f) N(H)-L″,
g) O-L″,
G″ represents
a) branched or straight-chain O—C₁-C₅ alkyl,
b) branched or straight-chain O—C₂-C₅ alkenyl,
c) branched or straight-chain O—C₁-C₅ alkyl-(O—C₁-C₄ alkyl)ₙ-O—C₁-C₄ alkyl,
d) branched or straight-chain O—C₂-C₅ alkynyl or
e) triphenylmethoxy,
R³ and R⁴ represent
a) hydrogen,
b) branched or straight-chain E-C₆-C₁₀ alkoxy,
c) branched or straight-chain E-C₆-C₁₀ alkyl,
d) branched or straight-chain E-C₆-C₁₀ alkenyl,
e) branched or straight-chain E-C₆-C₁₀ alkenyl,
f) hydroxyl,
g) branched or straight-chain C₁-C₅ alkyl or
h) branched or straight-chain C₁-C₅ alkoxy,
with the proviso that exactly one of the substituents R³ or R⁴ contains an E and the respective other substituent contains no E,
E represents a leaving group,
Q′ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
g) N(H)-2,2,2-trichloroethoxycarbonyl,
h) N(H)-1,1-dimethylpropynyl,
i) N(H)-1-methyl-1-phenylethoxycarbonyl,
j) N(H)-1-methyl-1-(4-biphenylyl)ethoxycarbonyl,
k) N(H)-cyclobutylcarbonyl,
l) N(H)-1-methylcyclobutylcarbonyl,
m) N(H)-vinylcarbonyl,
n) N(H)-allylcarbonyl,
o) N(H)-adamantylcarbonyl,
p) N(H)-diphenylmethylcarbonyl,
q) N(H)-cinnamylcarbonyl,
r) N(H)-formyl,
s) N(H)-benzoyl,
t) N(H)-trityl,
u) N(H)-p-methoxydiphenylmethyl,
v) N(H)-di(p-methoxyphenyl)phenylmethyl,
w) 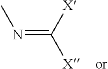
x) N-(tert-butoxycarbonyl)₂,
L″ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
X′ and X″ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
where n=0, 1, 2 or 3 and
all diastereomers and enantiomers are included.

7) Compounds of the formula (III) 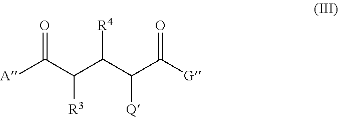
in which
A″ represents
a) branched or straight-chain C₁-C₅ alkoxy,
b) branched or straight-chain hydroxy C₁-C₅ alkoxy,
c) branched or straight-chain O—C₁-C₅ alkyl-(O—C₁-C₄ alkyl)ₙ-O—C₁-C₄ alkyl,
d) N(C₁-C₅ alkyl)₂,
e) NH₂,
f) N(H)-L″,
g) O-L″,
G″ represents
a) branched or straight-chain O—C₁-C₅ alkyl,
b) branched or straight-chain O—C₂-C₅ alkenyl,
c) branched or straight-chain O—C₁-C₅ alkyl-(O—C₁-C₄ alkyl)ₙ-O—C₁-C₄ alkyl,
d) branched or straight-chain O—C₂-C₅ alkynyl or
e) triphenylmethoxy,
R³ and R⁴ represent
a) hydrogen,
b) branched or straight-chain E-C₆-C₁₀ alkoxy,
c) branched or straight-chain E-C₆-C₁₀ alkyl,
d) branched or straight-chain E-C₆-C₁₀ alkenyl,
e) branched or straight-chain E-C₆-C₁₀ alkynyl,
f) hydroxyl,
g) branched or straight-chain C₁-C₅ alkyl or
h) branched or straight-chain C₁-C₅ alkoxy,
with the proviso that exactly one of the substituents R³ or R⁴ contains an E and the respective other substituent contains no E,
E represents a leaving group,
Q′ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
g) N(H)-2,2,2-trichloroethoxycarbonyl,
h) N(H)-1,1-dimethylpropynyl,
i) N(H)-1-methyl-1-phenylethoxycarbonyl,
j) N(H)-1-methyl-1-(4-biphenylyl)ethoxycarbonyl,
k) N(H)-cyclobutylcarbonyl,
l) N(H)-1-methylcyclobutylcarbonyl,
m) N(H)-vinylcarbonyl,
n) N(H)-allylcarbonyl,
o) N(H)-adamantylcarbonyl,
p) N(H)-diphenylmethylcarbonyl,
q) N(H)-cinnamylcarbonyl,
r) N(H)-formyl,
s) N(H)-benzoyl,
t) N(H)-trityl,
u) N(H)-p-methoxydiphenylmethyl,
v) N(H)-di(p-methoxyphenyl)phenylmethyl,
w) 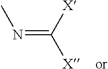
x) N-(tert-butoxycarbonyl)₂,
L″ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
X′ and X″ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
where n=0, 1, 2 or 3 and
all diastereomers and enantiomers are included.

8) A method comprising using the compounds of the formula (III) for preparing compounds of the formula (I) or (II): 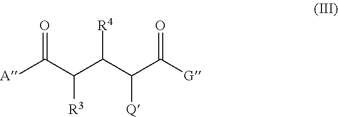
in which
A″ represents
a) branched or straight-chain C₁-C₅ alkoxy,
b) branched or straight-chain hydroxy C₁-C₅ alkoxy,
c) branched or straight-chain O—C₁-C₅ alkyl-(O—C₁-C₄ alkyl)ₙ-O—C₁-C₄ alkyl,
d) N(C₁-C₅ alkyl)₂,
e) NH₂,
f) N(H)-L″,
g) O-L″,
G″ represents
a) branched or straight-chain O—C₁-C₅ alkyl,
b) branched or straight-chain O—C₂-C₅ alkenyl,
c) branched or straight-chain O—C₁-C₅ alkyl-(O—C₁-C₄ alkyl)ₙ-O—C₁-C₄ alkyl,
d) branched or straight-chain O—C₂-C₅ alkynyl or
e) triphenylmethoxy,
R³ and R⁴ represent
a) hydrogen,
b) branched or straight-chain E-C₆-C₁₀ alkoxy,
c) branched or straight-chain E-C₆-C₁₀ alkyl,
d) branched or straight-chain E-C₆-C₁₀ alkenyl,
e) branched or straight-chain E-C₆-C₁₀ alkynyl,
f) hydroxyl,
g) branched or straight-chain C₁-C₅ alkyl or
h) branched or straight-chain C₁-C₅ alkoxy,
with the proviso that exactly one of the substituents R³ or R⁴ contains an E and the respective other substituent contains no E,
E represents a leaving group,
Q′ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
g) N(H)-2,2,2-trichloroethoxycarbonyl,
h) N(H)-1,1-dimethylpropynyl,
i) N(H)-1-methyl-1-phenylethoxycarbonyl,
j) N(H)-1-methyl-1-(4-biphenylyl)ethoxycarbonyl,
k) N(H)-cyclobutylcarbonyl,
l) N(H)-1-methylcyclobutylcarbonyl,
m) N(H)-vinylcarbonyl,
n) N(H)-allylcarbonyl,
o) N(H)-adamantylcarbonyl,
p) N(H)-diphenylmethylcarbonyl,
q) N(H)-cinnamylcarbonyl,
r) N(H)-formyl,
s) N(H)-benzoyl,
t) N(H)-trityl,
u) N(H)-p-methoxyphenyldiphenylmethyl,
v) N(H)-di(p-methoxyphenyl)phenylmethyl,
w) 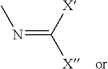
x) N-(tert-butoxycarbonyl)₂,
L″ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
X′ and X″ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
where n=0, 1, 2 or 3 and
all diastereomers and enantiomers are included.

9) Compounds of the formula (IV) 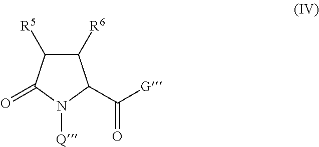
in which
G′″ represents
a) branched or straight-chain O—C₁-C₅ alkyl,
b) branched or straight-chain O—C₂-C₅ alkenyl,
c) branched or straight-chain O—C₁-C₅ alkyl)ₙ-O—C₁-C₄ alkyl,
d) branched or straight-chain O—C₂-C₅ alkynyl or
e) triphenylmethoxy,
R⁵ and R⁶ represent
a) hydrogen,
b) hydroxyl,
c) branched or straight-chain C₁-C₅ alkyl,
d) branched or straight-chain C₁-C₅ alkoxy or
e) R⁷-E′,
with the proviso that exactly one of the substituents R⁵ or R⁶ contains an E′ and the respective other substituent contains no E′,
E′ represents a leaving group
R⁷ represents
a) branched or straight-chain C₆-C₁₀ alkoxy,
b) branched or straight-chain C₆-C₁₀ alkyl,
c) branched or straight-chain C₆-C₁₀ alkenyl or
d) branched or straight-chain C₆-C₁₀ alkynyl,
Q′″ represents
a) N-tert-butoxycarbonyl
b) N-allyloxycarbonyl,
c) N-benzyloxycarbonyl,
d) N-ethoxycarbonyl,
e) N-methoxycarbonyl,
f) N-propoxycarbonyl,
g) N-2,2,2-trichloroethoxycarbonyl,
h) hydrogen,
i) N-1-methyl-1-phenylethoxycarbonyl,
j) N-1-methyl-1-(4-biphenylyl)ethoxycarbonyl,
k) N-cyclobutylcarbonyl,
l) N-1-methylcyclobutylcarbonyl,
m) N-vinylcarbonyl,
n) N-allylcarbonyl,
o) N-adamantylcarbonyl,
p) N-diphenylmethylcarbonyl,
q) N-cinnamylcarbonyl,
r) N-formyl,
s) N-benzoyl,
t) N(H)-trityl,
u) N(H)-p-methoxyphenyldiphenylmethyl,
v) N(H)-di(p-methoxyphenyl)phenylmethyl,
w) 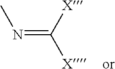
x) N-(tert-butoxycarbonyl)₂,
X′″ and X″″ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
where n=0, 1, 2 or 3 and
all diastereomers and enantiomers are included.

10) A method comprising using compounds of the formula (VI) for preparing compounds of the formula (I) or (II): 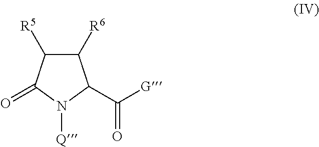
in which
G′″ represents
a) branched or straight-chain O—C₁-C₅ alkyl,
b) branched or straight-chain O—C₂-C₅ alkenyl,
c) branched or straight-chain O—C₁-C₅ alkyl-(O—C₁-C₄ alkyl)ₙ-O—C₁-C₄ alkyl,
d) branched or straight-chain O—C₂-C₅ alkynyl or
e) triphenylmethoxy,
R⁵ and R⁶ represent
a) hydrogen,
b) hydroxyl,
c) branched or straight-chain C₁-C₅ alkyl,
d) branched or straight-chain C₁-C₅ alkoxy or
e) R⁷-E′,
with the proviso that exactly one of the substituents R⁵ or R⁶ contains an E′ and the respective other substituent contains no E′,
E′ represents a leaving group
R⁷ represents
a) branched or straight-chain C₆-C₁₀ alkoxy,
b) branched or straight-chain C₆-C₁₀ alkyl,
c) branched or straight-chain C₆-C₁₀ alkenyl or
d) branched or straight-chain C₆-C₁₀ alkynyl,
Q′″ represents
a) N-tert-butoxycarbonyl
b) N-allyloxycarbonyl,
c) N-benzyloxycarbonyl,
d) N-ethoxycarbonyl,
e) N-methoxycarbonyl,
f) N-propoxycarbonyl,
g) N-2,2,2-trichloroethoxycarbonyl,
h) hydrogen,
i) N-1-methyl-1-phenylethoxycarbonyl,
j) N-1-methyl-1-(4-biphenylyl)ethoxycarbonyl,
k) N-cyclobutylcarbonyl,
l) N-1-methylcyclobutylcarbonyl,
m) N-vinylcarbonyl,
n) N-allylcarbonyl,
o) N-adamantylcarbonyl,
p) N-diphenylmethylcarbonyl,
q) N-cinnamylcarbonyl,
r) N-formyl,
s) N-benzoyl,
t) N(H)-trityl,
u) N(H)-p-methoxyphenyldiphenylmethyl,
v) N(H)-di(p-methoxyphenyl)phenylmethyl,
w) 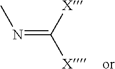
x) N-(tert-butoxycarbonyl)₂,
X′ and X″″ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
where n=0, 1, 2 or 3 and
all diastereomers and enantiomers are included.

11) Imaging kit, comprising compounds of the general formula III or IV.

12) Pharmaceutical composition, comprising compounds of the general formula I, II, III or IV and suitable pharmaceutical carrier substances.

13) Compounds of the general formula V 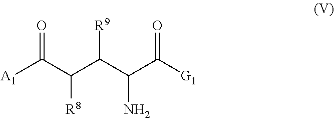
in which
A₁ represents
a) hydroxyl,
b) branched or straight-chain C₁-C₅ alkoxy,
c) branched or straight-chain hydroxy C₁-C₅ alkoxy,
d) branched or straight-chain O—C₁-C₅ alkyl-(O—C₁-C₄ alkyl)ₙ-O—C₁-C₄ alkyl,
ed) N(C₁-C₅ alkyl)₂,
f) NH₂,
g) N(H)-L₁,
h) O-L₁ or
i) O—Z₁,
G₁ represents
a) hydroxyl,
b) O—Z₁,
c) branched or straight-chain O—C₁-C₅ alkyl,
d) branched or straight-chain O—C₁-C₅ alkenyl,
e) branched or straight-chain O—C₁-C₅ alkyl-(O—C₁-C₄ alkyl)ₙ-O—C₁-C₄ alkyl,
f) branched or straight-chain O—C₁-C₅ alkynyl or
g) triphenylmethoxy,
R⁸ and R⁹ represent
a) hydrogen,
b) substituted or unsubstituted ¹⁸F—C₆-C₁₀ mono- or bicyclic aryl,
c) substituted or unsubstituted alkylene-C₆-C₁₀ mono- or bicyclic aryl-¹⁸F,
d) substituted or unsubstituted ¹⁸F—C₅-C₁₀ mono- or bicyclic heteroaryl,
e) substituted or unsubstituted alkylene-C₅-C₁₀ mono- or bicyclic heteroaryl-¹⁸F,
f) substituted or unsubstituted ¹⁸F—C₃-C₆ cycloalkyl,
g) substituted or unsubstituted alkylene-C₃-C₆ cycloalkyl-¹⁸F,
h) hydroxyl,
i) branched or straight-chain C₁-C₅ alkyl,
j) branched or straight-chain C₁-C₅ alkoxy,
k) substituted or unsubstituted O-alkylene-C₆-C₁₀ mono- or bicyclic aryl-¹⁸F,
l) substituted or unsubstituted O-alkylene-C₅-C₁₀ mono- or bicyclic heteroaryl-¹⁸F or
m) substituted or unsubstituted O—C₃-C₆ cycloalkyl-¹⁸F,
alkylene being optionally interrupted by one or more O, S or N,
with the proviso that one of the substituents R⁸ or R⁹ contains exactly one ¹⁸F isotope and the respective other substituent contains no ¹⁸F isotope,
L₁ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
Z₁ represents a metal cation equivalent,
where n=0, 1, 2 or 3 and
all diastereomers and enantiomers are included.

14) Compounds according to claim 13, selected from the group of compounds of the formulae:

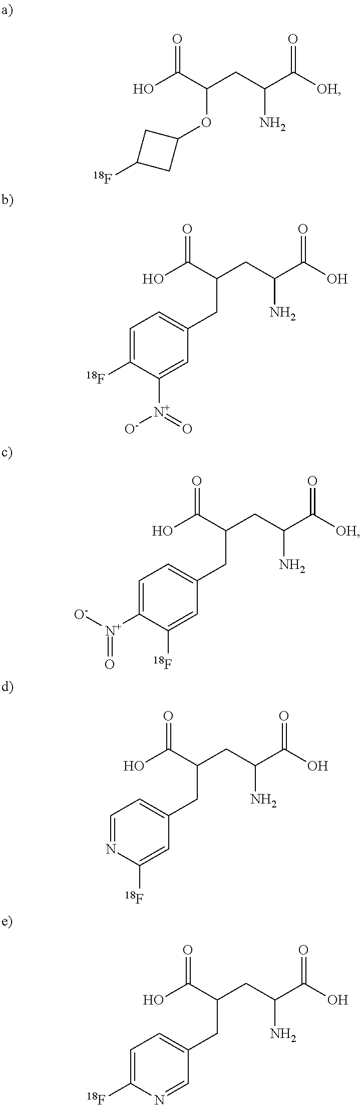

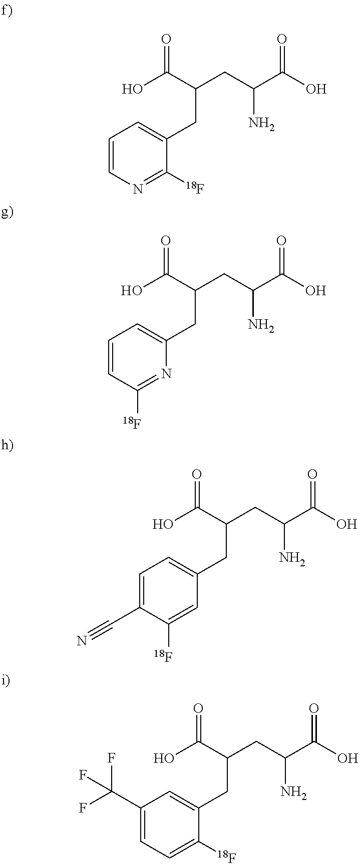

15) Process for preparing compounds of the general formula (V) according to claim 13,
which comprises
removing one or more protective groups present in a compound of the formula (VI) 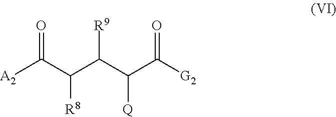
in which
A₂ represents
a) hydroxyl,
b) branched or straight-chain C₁-C₅ alkoxy,
c) branched or straight-chain hydroxy C₁-C₅ alkoxy,
d) branched or straight-chain O—C₁-C₅ alkyl-(O—C₁-C₄ alkyl)ₙ-O—C₁-C₄ alkyl,
e) N(C₁-C₅ alkyl)₂,
f) NH₂,
g) N(H)-L₂ or
h) O-L₂
G₂ represents
a) hydroxyl,
b) O—Z₂,
c) branched or straight-chain O—C₁-C₅ alkyl,
d) branched or straight-chain O—C₂-C₅ alkenyl,
e) branched or straight-chain O—C₁-C₅ alkyl-(O—C₁-C₄ alkyl)ₙ-O—C₁-C₄ alkyl,
f) branched or straight-chain O—C₂-C₅ alkynyl or
g) triphenylmethoxy,
R⁸ and R⁹ represent
a) hydrogen,
b) substituted or unsubstituted ¹⁸F—C₆-C₁₀ mono- or bicyclic aryl,
c) substituted or unsubstituted alkylene-C₆-C₁₀ mono- or bicyclic aryl-¹⁸F,
d) substituted or unsubstituted ¹⁸F—C₅-C₁₀ mono- or bicyclic heteroaryl,
e) substituted or unsubstituted alkylene-C₅-C₁₀ mono- or bicyclic heteroaryl-¹⁸F,
f) substituted or unsubstituted ¹⁸F—C₃-C₆ cycloalkyl,
g) substituted or unsubstituted alkylene-C₃-C₆ cycloalkyl-¹⁸F
h) hydroxyl,
i) branched or straight-chain C₁-C₅ alkyl,
j) branched or straight-chain C₁-C₅ alkoxy,
k) substituted or unsubstituted O-alkylene-C₆-C₁₀ mono- or bicyclic aryl-¹⁸F,
l) substituted or unsubstituted O-alkylene-C₅-C₁₀ mono- or bicyclic heteroaryl-¹⁸F or
m) substituted or unsubstituted O—C₃-C₆ cycloalkyl-¹⁸F,
alkylene being optionally interrupted by one or more O, S or N,
with the proviso that one of the substituents R₈ or R⁹ contains exactly one ¹⁸F isotope and the respective other substituent contains no ¹⁸F isotope,
Q₁ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
e) N(H)-2,2,2-trichloroethoxycarbonyl,
f) N(H)-1,1-dimethylpropynyl,
g) N(H)-1-methyl-1-phenylethoxycarbonyl,
h) N(H)-1-methyl-1-(4-biphenylyl)ethoxycarbonyl,
i) N(H)-cyclobutylcarbonyl,
j) N(H)-1-methylcyclobutylcarbonyl,
k) N(H)-vinylcarbonyl,
l) N(H)-allylcarbonyl,
m) N(H)-adamantylcarbonyl,
n) N(H)-diphenylmethylcarbonyl,
o) N(H)-cinnamylcarbonyl,
p) N(H)-formyl,
q) N(H)-benzoyl,
r) N(H)-trityl,
s) N(H)-p-methoxyphenyldiphenylmethyl,
t) N(H)-di(p-methoxyphenyl)phenylmethyl,
u) 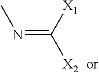
v) N-(tert-butoxycarbonyl)₂,
L₂ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) or straight-chain C₂-C₅ alkynyl,
X₁ and X independently of one another represent
a) branched or straight-chain C₁-C₅ alkyl,
b) substituted or unsubstituted aryl,
c) substituted or unsubstituted arylalkyl or
d) substituted or unsubstituted heteroaryl,
Z₂ represents a metal cation equivalent,
where n=0, 1, 2 or 3 and
all diastereomers and enantiomers are included.

16) Compounds of the general formula (VI): 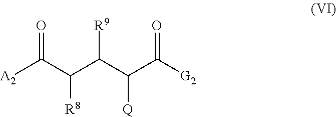
in which
A₂ represents
a) hydroxyl,
b) branched or straight-chain C₁-C₅ alkoxy,
c) branched or straight-chain hydroxy C₁-C₅ alkoxy,
d) branched or straight-chain O—C₁-C₅ alkyl-(O—C₁-C₄ alkyl)ₙ-O—C₁-C₄ alkyl,
e) N(C₁-C₅ alkyl)₂,
f) NH₂,
g) N(H)-L₂ or
h) O-L₂
G₂ represents
a) hydroxyl,
b) O—Z₂,
c) branched or straight-chain O—C₁-C₅ alkyl,
d) branched or straight-chain O—C₂-C₅ alkenyl,
e) branched or straight-chain O—C₁-C₅ alkyl-(O—C₁-C₄ alkyl)ₙ-O—C₁-C₄ alkyl,
f) branched or straight-chain O—C₂-C₅ alkynyl or
g) triphenylmethoxy,
R⁸ and R⁹ represent
a) hydrogen,
b) substituted or unsubstituted ¹⁸F—C₆-C₁₀ mono- or bicyclic aryl,
c) substituted or unsubstituted alkylene-C₆-C₁₀ mono- or bicyclic aryl-¹⁸F,
d) substituted or unsubstituted ¹⁸F—C₅-C₁₀ mono- or bicyclic heteroaryl,
e) substituted or unsubstituted alkylene-C₅-C₁₀ mono- or bicyclic heteroaryl-¹⁸F,
f) substituted or unsubstituted ¹⁸F—C₃-C₆ cycloalkyl,
g) substituted or unsubstituted alkylene-C₃-C₆ cycloalkyl-¹⁸F,
h) hydroxyl,
i) branched or straight-chain C₁-C₅ alkyl,
j) branched or straight-chain C₁-C₅ alkoxy,
k) substituted or unsubstituted O-alkylene-C₆-C₁₀ mono- or bicyclic aryl-¹⁸F,
l) substituted or unsubstituted O-alkylene-C₅-C₁₀ mono- or bicyclic heteroaryl-¹⁸F or
m) substituted or unsubstituted O—C₃-C₆ cycloalkyl-¹⁸F,
alkylene being optionally interrupted by one or more O, S or N,
with the proviso that one of the substituents R₈ or R⁹ contains exactly one ¹⁸F isotope and the respective other substituent contains no ¹⁸F isotope,
Q₁ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
e) N(H)-2,2,2-trichloroethoxycarbonyl,
f) N(H)-1,1-dimethylpropynyl,
g) N(H)-1-methyl-1-phenylethoxycarbonyl,
h) N(H)-1-methyl-1-(4-biphenylyl)ethoxycarbonyl,
i) N(H)-cyclobutylcarbonyl,
j) N(H)-1-methylcyclobutylcarbonyl,
k) N(H)-vinylcarbonyl,
l) N(H)-allylcarbonyl,
m) N(H)-adamantylcarbonyl,
n) N(H)-diphenylmethylcarbonyl,
o) N(H)-cinnamylcarbonyl,
p) N(H)-formyl,
q) N(H)-benzoyl,
r) N(H)-trityl,
s) N(H)-p-methoxyphenyldiphenylmethyl,
t) N(H)-di(p-methoxyphenyl)phenylmethyl,
u) 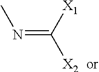
v) N-(tert-butoxycarbonyl)₂,
L₂ represents
a) branched or straight-chain C₁-C₅ alkyl,
b) branched or straight-chain C₂-C₅ alkenyl,
c) branched or straight-chain C₁-C₄ alkyl)ₙ-O—C₁-C₄ alkyl or
d) branched or straight-chain C₂-C₅ alkynyl,
X₁ and X₂ independently of one another represent
a) branched or straight-chain C₁-C₅ alkyl,
b) substituted or unsubstituted aryl,
c) substituted or unsubstituted arylalkyl or
d) substituted or unsubstituted heteroaryl,
Z₂ represents a metal cation equivalent,
where n=0, 1, 2 or 3 and
all diastereomers and enantiomers are included.

17) Process for preparing compounds of the general formula (VI) according to claim 16,
which comprises
reacting a compound of the formula (VII) with F-18 fluoride 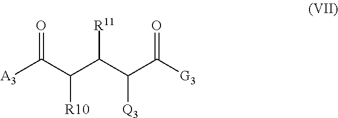
in which
A represents
a) branched or straight-chain C₁-C₅ alkoxy,
b) branched or straight-chain hydroxy C₁-C₅ alkoxy,
c) branched or straight-chain O—C₁-C₅ alkyl-(O—C₁-C₄ alkyl)ₙ-O—C₁-C₄ alkyl,
d) N(C₁-C₅ alkyl)₂,
e) NH₂
f) N(H)-L₃ or
g) O-L₃,
G₃ represents
a) O—Z₃,
b) branched or straight-chain O—C₁-C₅alkyl,
c) branched or straight-chain O—C₂-C₅ alkenyl,
d) branched or straight-chain O—C₁-C₅ alkyl-(O—C₁-C₄ alkyl)ₙ-O—C₁-C₄ alkyl,
e) branched or straight-chain O—C₁-C₅ alkynyl or
f) triphenylmethoxy,
R¹⁰ and R¹¹ represent
a) hydrogen,
b) substituted or unsubstituted E₁-C₆-C₁₀ mono- or bicyclic aryl,
c) substituted or unsubstituted alkylene-C₆-C₁₀ mono- or bicyclic aryl-E₁,
d) substituted or unsubstituted E₁-C₅-C₁₀ mono- or bicyclic heteroaryl,
e) substituted or unsubstituted alkylene-C₅-C₁₀ mono- or bicyclic heteroaryl-E₁,
f) substituted or unsubstituted E₁-C₃-C₆ cycloalkyl,
g) substituted or unsubstituted alkylene-C₃-C₆ cycloalkyl-E₁,
h) hydroxyl,
i) branched or straight-chain C₁-C₅ alkyl,
j) branched or straight-chain C₁-C₅ alkoxy,
k) substituted or unsubstituted O-alkylene-C₆-C₁₀ mono- or bicyclic aryl-¹⁸F,
l) substituted or unsubstituted O-alkylene-C₅-C₁₀ mono- or bicyclic heteroaryl-¹⁸F or
m) substituted or unsubstituted O—C₃-C₆ cycloalkyl-¹⁸F,
alkylene being optionally interrupted by one or more O, S or N,
with the proviso that exactly one of the substituents R¹⁰ or R¹¹ contains an E₁ and the respective other substituent contains no E₁,
E₁ represents a leaving group,
Q₂ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
N(H)-propoxycarbonyl,
g) N(H)-2,2,2-trichloroethoxycarbonyl,
h) N(H)-1,1-dimethylpropynyl,
i) N(H)-1-methyl-1-phenylethoxycarbonyl,
j) N(H)-1-methyl-1-(4-biphenylyl)ethoxycarbonyl,
k) N(H)-cyclobutylcarbonyl,
l) N(H)-1-methylcyclobutylcarbonyl,
m) N(H)-vinylcarbonyl,
n) N(H)-allylcarbonyl,
o) N(H)-adamantylcarbonyl,
p) N(H)-diphenylmethylcarbonyl,
q) N(H)-cinnamylcarbonyl,
r) N(H)-formyl,
s) N(H)-benzoyl,
t) N(H)-trityl,
u) N(H)-p-methoxyphenyldiphenylmethyl,
v) N(H)-di(p-methoxyphenyl)phenylmethyl,
w) 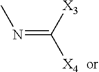
x) N-(tert-butoxycarbonyl)₂,
L₃ represents
a) branched or straight-chain C₁-C₅ alkyl,
b) branched or straight-chain C₁-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
X₃ and X₄ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
Z₃ represents a metal cation equivalent,
where n=0, 1, 2 or 3 and
all diastereomers and enantiomers are included.

18) Compounds of the formula (VII) 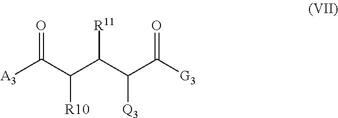
in which
A₃ represents
a) branched or straight-chain C₁-C₅ alkoxy,
b) branched or straight-chain hydroxy C₁-C₅ alkoxy,
c) branched or straight-chain O—C₁-C₅ alkyl-(O—C₁-C₄ alkyl)ₙ-O—C₁-C₄ alkyl,
d) N(C₁-C₅ alkyl)₂,
e) NH₂,
f) N(H)-L₃ or
g) O-L₃,
G₃ represents
a) O—Z₃,
b) branched or straight-chain O—C₁-C₅ alkyl,
c) branched or straight-chain O—C₂-C₅ alkenyl,
d) branched or straight-chain O—C₁-C₅ alkyl-(O—C₁-C₄ alkyl)ₙ-O—C₁-C₄ alkyl,
e) branched or straight-chain O—C₁-C₅ alkynyl or
f) triphenylmethoxy,
R¹⁰ and R¹¹ represent
a) hydrogen,
b) substituted or unsubstituted E₁-C₆-C₁₀ mono- or bicyclic aryl,
c) substituted or unsubstituted alkylene-C₆-C₁₀ mono- or bicyclic aryl-E₁,
d) substituted or unsubstituted E₁-C₅-C₁₀ mono- or bicyclic heteroaryl,
e) substituted or unsubstituted alkylene-C₅-C₁₀ mono- or bicyclic heteroaryl-E₁,
f) substituted or unsubstituted E₁-C₃-C₆ cycloalkyl,
g) substituted or unsubstituted alkylene-C₃-C₆ cycloalkyl-E₁,
h) hydroxyl,
i) branched or straight-chain C₁-C₅ alkyl,
j) branched or straight-chain C₁-C₅ alkoxy,
k) substituted or unsubstituted O-alkylene-C₆-C₁₀ mono- or bicyclic aryl-¹⁸F,
l) substituted or unsubstituted O-alkylene-C₅-C₁₀ mono- or bicyclic heteroaryl-¹⁸F or
m) substituted or unsubstituted O—C₃-C₆ cycloalkyl-¹⁸F,
alkylene being optionally interrupted by one or more O, S or N,
with the proviso that exactly one of the substituents R¹⁰ or R¹¹ contains an E₁ and the respective other substituent contains no E₁,
E₁ represents a leaving group,
Q₂ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
f) N(H)-propoxycarbonyl,
g) N(H)-2,2,2-trichloroethoxycarbonyl,
h) N(H)-1,1-dimethylpropynyl,
i) N(H)-1-methyl-1-phenylethoxycarbonyl,
j) N(H)-1-methyl-1-(4-biphenylyl)ethoxycarbonyl,
k) N(H)-cyclobutylcarbonyl,
l) N(H)-1-methylcyclobutylcarbonyl,
m) N(H)-vinylcarbonyl,
n) N(H)-allylcarbonyl,
o) N(H)-adamantylcarbonyl,
p) N(H)-diphenylmethylcarbonyl,
q) N(H)-cinnamylcarbonyl,
r) N(H)-formyl,
s) N(H)-benzoyl,
t) N(H)-trityl,
u) N(H)-p-methoxyphenyldiphenylmethyl,
v) N(H)-di(p-methoxyphenyl)phenylmethyl,
w) 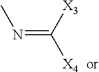
x) N-(tert-butoxycarbonyl)₂,
L₃ represents
a) branched or straight-chain C₁-C₅, alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
X₃ and X₄ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
Z₃ represents a metal cation equivalent,
where n=0, 1, 2 or 3 and
all diastereomers and enantiomers are included.

19) A method comprising using compounds of the formula (VII) for preparing compounds of the formula (V) or (VI): 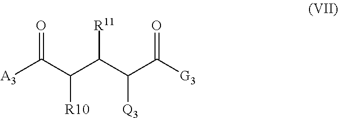
in which
A₃ represents
a) branched or straight-chain C₁-C₅ alkoxy,
b) branched or straight-chain hydroxy C₁-C₅ alkoxy,
c) branched or straight-chain O—C₁-C₅ alkyl-(O—C₁-C₄ alkyl)ₙ-O—C₁-C₄ alkyl,
d) N(C₁-C₅ alkyl)₂,
e) NH₂,
N(H)-L₃ or
g) O-L₃,
G₃ represents
a) O—Z₃,
b) branched or straight-chain O—C₁-C₅ alkyl,
c) branched or straight-chain O—C₂-C₅ alkenyl,
d) branched or straight-chain O—C₁-C₅ alkyl-(O—C₁-C₄ alkyl)ₙ-O—C₁-C₄ alkyl,
e) branched or straight-chain O—C₁-C₅ alkynyl or
f) triphenylmethoxy,
R¹⁰ and R¹¹ represent
a) hydrogen,
b) substituted or unsubstituted E₁-C₆-C₁₀ mono- or bicyclic aryl,
c) substituted or unsubstituted alkylene-C₆-C₁₀ mono- or bicyclic aryl-E₁,
d) substituted or unsubstituted E₁-C₅-C₁₀ mono- or bicyclic heteroaryl,
e) substituted or unsubstituted alkylene-C₅-C₁₀ mono- or bicyclic heteroaryl-E₁,
f) substituted or unsubstituted E₁-C₃-C₆ cycloalkyl,
g) substituted or unsubstituted alkylene-C₃-C₆ cycloalkyl-E₁,
h) hydroxyl,
i) branched or straight-chain C₁-C₅ alkyl,
j) branched or straight-chain C₁-C₅ alkoxy,
k) substituted or unsubstituted O-alkylene-C₆-C₁₀ mono- or bicyclic aryl-E₁,
l) substituted or unsubstituted O-alkylene-C₅-C₁₀ mono- or bicyclic heteroaryl-E₁ or
m) substituted or unsubstituted O—C₃-C₆ cycloalkyl-E₁,
alkylene being optionally interrupted by one or more O, S or N,
with the proviso that exactly one of the substituents R¹⁰ or R¹¹ contains an E₁ and the respective other substituent contains no E₁,
E₁ represents a leaving group,
Q₂ represents
a) N(H)-tert-butoxycarbonyl
b) N(H)-allyloxycarbonyl,
c) N(H)-benzyloxycarbonyl,
d) N(H)-ethoxycarbonyl,
e) N(H)-methoxycarbonyl,
N(H)-propoxycarbonyl,
g) N(H)-2,2,2-trichloroethoxycarbonyl,
h) N(H)-1,1-di methylpropynyl,
i) N(H)-1-methyl-1-phenylethoxycarbonyl,
j) N(H)-1-methyl-1-(4-biphenylyl)ethoxycarbonyl,
k) N(H)-cyclobutylcarbonyl,
l) N(H)-1-methylcyclobutylcarbonyl,
m) N(H)-vinylcarbonyl,
n) N(H)-allylcarbonyl,
o) N(H)-adamantylcarbonyl,
p) N(H)-diphenylmethylcarbonyl,
q) N(H)-cinnamylcarbonyl,
r) N(H)-formyl,
s) N(H)-benzoyl,
t) N(H)-trityl,
u) N(H)-p-methoxyphenyldiphenylmethyl,
v) N(H)-di(p-methoxyphenyl)phenylmethyl,
w) 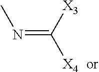
x) N-(tert-butoxycarbonyl)₂,
L₃ represents
a) branched or straight-chain C₁-C₅ alkyl,
b) branched or straight-chain C₂-C₅ alkenyl,
c) branched or straight-chain C₁-C₅ alkyl-(O—C₁-C₄ alkyl)ₙ-O—C₁-C₄ alkyl or
d) branched or straight-chain C₂-C₅ alkynyl,
X₃ and X₄ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
Z₃ represents a metal cation equivalent,
where n=0, 1, 2 or 3 and
all diastereomers and enantiomers are included.

20) Compounds of the formula (VIII) 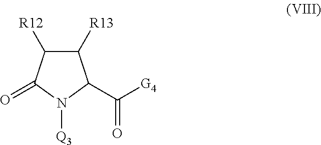
in which
G₄ represents
a) branched or straight-chain O—C₁-C₅ alkyl,
b) branched or straight-chain O—C₂-C₅ alkenyl,
c) branched or straight-chain O—C₁-C₅ alkyl-(O—C₁-C₄ alkyl)ₙ-O—C₁-C₄ alkyl,
d) branched or straight-chain O—C₂-C₅ alkynyl or
e) triphenylmethoxy,
R¹² and R¹³ represent
a) hydrogen,
b) hydroxyl,
c) branched or straight-chain C₁-C₅ alkyl,
d) branched or straight-chain C₁-C₅ alkoxy or
e) R¹⁴-E₂,
with the proviso that exactly one of the substituents R¹² or R¹³ contains an E₂ and the respective other substituent contains no E₂,
E₂ represents a leaving group,
R¹⁴ represents
a) substituted or unsubstituted C₆-C₁₀ mono- or bicyclic aryl,
b) substituted or unsubstituted alkylene-C₆-C₁₀ mono- or bicyclic
aryl,
c) substituted or unsubstituted C₅-C₁₀ mono- or bicyclic heteroaryl,
d) substituted or unsubstituted alkylene-C₅-C₁₀ mono- or bicyclic heteroaryl,
f) substituted or unsubstituted C₃-C₆ cycloalkyl,
g) substituted or unsubstituted alkylene-C₃-C₆ cycloalkyl,
h) substituted or unsubstituted O-alkylene C₅-C₁₀ mono- or bicyclic aryl,
i) substituted or unsubstituted O-alkylene C₅-C₁₀ mono- or bicyclic heteroaryl or
j) substituted or unsubstituted O—C₃-C₆ cycloalkyl,
alkylene being optionally interrupted by one or more O, S or N,
Q₃ represents
a) N-tert-butoxycarbonyl
b) N-allyloxycarbonyl,
c) N-benzyloxycarbonyl,
d) N-ethoxycarbonyl,
e) N-methoxycarbonyl,
f) N-propoxycarbonyl,
g) N-2,2,2-trichloroethoxycarbonyl,
h) hydrogen,
i) N-1-methyl-1-phenylethoxycarbonyl,
j) N-1-methyl-1-(4-biphenylyl)ethoxycarbonyl,
k) N-cyclobutylcarbonyl,
l) N-1-methylcyclobutylcarbonyl,
m) N-vinylcarbonyl,
n) N-allylcarbonyl,
o) N-adamantylcarbonyl,
p) N-diphenylmethylcarbonyl,
q) N-cinnamylcarbonyl,
r) N-formyl or
s) N-benzoyl,
t) N(H)-trityl,
u) N(H)-p-methoxyphenyldiphenylmethyl,
v) N(H)-di(p-methoxyphenyl)phenylmethyl,
w) 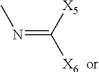
x) N-(tert-butoxycarbonyl)₂,
X₅ and X₆ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
where n=0, 1, 2 or 3 and
all diastereomers and enantiomers are included.

21) A method comprising using compounds of the formula (VIII) for preparing compounds of the formula (V) or (VI): 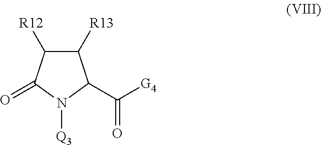
in which
G₄ represents
a) branched or straight-chain O—C₁-C₅ alkyl,
b) branched or straight-chain O—C₂-C₅ alkenyl,
c) branched or straight-chain O—C₁-C₅ alkyl-(O—C₁-C₄ alkyl)ₙ-O—C₁-C₄ alkyl,
d) branched or straight-chain O—C₂-C₅ alkynyl or
e) triphenylmethoxy,
R¹² and R¹³ represent
a) hydrogen,
b) hydroxyl,
c) branched or straight-chain C₁-C₅ alkyl,
d) branched or straight-chain C₁-C₅ alkoxy or
e) R¹⁴-E₂,
with the proviso that exactly one of the substituents R¹² or R¹³ contains an E₂ and the respective other substituent contains no E₂,
E₂ represents a leaving group,
R₁₄ represents
a) substituted or unsubstituted C₆-C₁₀ mono- or bicyclic aryl,
b) substituted or unsubstituted alkylene-C₆-C₁₀ mono- or bicyclic aryl,
c) substituted or unsubstituted C₅-C₁₀ mono- or bicyclic heteroaryl,
d) substituted or unsubstituted alkylene-C₅-C₁₀ mono- or bicyclic heteroaryl,
e) substituted or unsubstituted C₃-C₆ cycloalkyl,
f) substituted or unsubstituted alkylene-C₃-C₆ cycloalkyl
g) substituted or unsubstituted O-alkylene C₅-C₁₀ mono- or bicyclic aryl,
h) substituted or unsubstituted O-alkylene-C₅-C₁₀ mono- or bicyclic heteroaryl or
i) substituted or unsubstituted O—C₃-C₆ cycloalkyl,
alkylene being optionally interrupted by one or more O, S or N,
Q₃ represents
a) N-tert-butoxycarbonyl
b) N-allyloxycarbonyl,
c) N-benzyloxycarbonyl,
d) N-ethoxycarbonyl,
e) N-methoxycarbonyl,
0 N-propoxycarbonyl,
g) N-2,2,2-trichloroethoxycarbonyl,
h) hydrogen,
i) N-1-methyl-1-phenylethoxycarbonyl,
j) N-1-methyl-1-(4-biphenylyl)ethoxycarbonyl,
k) N-cyclobutylcarbonyl,
l) N-1-methylcyclobutylcarbonyl,
m) N-vinylcarbonyl,
n) N-allylcarbonyl,
o) N-adamantylcarbonyl,
p) N-diphenylmethylcarbonyl,
q) N-cinnamylcarbonyl,
r) N-formyl,
s) N-benzoyl,
t) N(H)-trityl,
u) N(H)-p-methoxyphenyldiphenylmethyl,
v) N(H)-di(p-methoxyphenyl)phenylmethyl,
w) 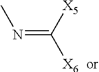
x) N-(tert-butoxycarbonyl)₂,
X₅ and X₆ independently of one another represent
a) branched or straight-chain C₁-C₅ alkyl,
b) substituted or unsubstituted aryl,
c) substituted or unsubstituted alkylaryl or
d) substituted or unsubstituted heteroaryl,
where n=0, 1, 2 or 3 and
all diastereomers and enantiomers are included.

22) Imaging-kit, comprising compounds of the general formula VII or VIII.

23) Pharmaceutical composition, comprising compounds of the general formula V, VI, VII or VIII and suitable pharmaceutical carrier substances.

24) Compounds according to claim 1 for use as a medicament.

25) Compounds according to claim 1 for use for imaging in tumour disorders.

26) Compounds according to claim 1 for producing a medicament for imaging in tumour disorders.

27) Compounds according to claim 1, characterized in that the compounds are suitable for imaging in a dosage range of 37-600 MBq.

28) Compounds according to claim 27, characterized in that the compounds are particularly suitable in a dosage range of 150 MBq-370 MBq.
